(19) United States
(12) Patent Application Publication
(10) Pub. No.: US 2026/0041674 A1
(43) Pub. Date: Feb. 12, 2026

(54) METHODS AND COMPOSITIONS FOR MODULATING THE ACTIVITY OF A DIMERIZING AGENT REGULATED IMMUNOMODULATORY COMPLEX

(71) Applicants: Seattle Children’s Hospital d/b/a Seattle Children’s Research Institute, Seattle, WA (US); Regeneron Pharmaceuticals, Inc., Tarrytown, NY (US)

(72) Inventors: Jacob S. Appelbaum, Seattle, WA (US); Rebecca Gardner, Seattle, WA (US); Joshua Gustafson, Seattle, WA (US); Michael C. Jensen, Bainbridge Island, WA (US); James Brian Rottman, Sudbury, MA (US); Mark Pogson, North Bend, WA (US); Jordan Jarjour, Seattle, WA (US); Alexander Astrakhan, Seattle, WA (US)

(73) Assignees: Seattle Children's Hospital d/b/a Seattle Children's Research Institute, Seattle, WA (US); Regeneron Pharmaceuticals, Inc., Tarrytown, NY (US)

(21) Appl. No.: 18/718,159

(22) PCT Filed: Dec. 9, 2022
(86) PCT No.: PCT/US2022/081322

§ 371 Date: Aug. 4, 2025

Related U.S. Application Data
(60) Provisional application No. 63/288,468, filed on Dec. 10, 2021.

(51) Int. Cl. A61K 31/436; A61K 31/675; A61K 31/7076; A61P 35/00; G01N 33/574

(52) U.S. Cl. CPC A61K 31/436; A61K 31/675; A61K 31/7076; A61P 35/00; G01N 33/57484; G01N 2333/70503

(57) ABSTRACT

Methods and compositions for priming a dimerizing agent regulated immunomodulatory complex for signaling by inducing the multimerization of at least a first fusion protein and a second fusion protein to thereby form the dimerizing agent regulated immunomodulatory complex are described. The methods and compositions utilize dimerizing agent dosing schedules designed to: (i) maintain specified blood trough levels of the dimerizing agent, (ii) allow activation of the immunomodulatory complex; (iii) reduce or avoid potential immunosuppressive effects of the dimerizing agent, (iv) reduce or avoid immune cell exhaustion, and/or (v) reduce or avoid side effects associated with activation of the immunomodulatory complex.

### CROSS REFERENCE TO RELATED APPLICATIONS

This application is a U.S. National Phase patent application based on International Patent Application No. PCT/US2022/081322, filed on Dec. 9, 2022, which claims priority to U.S. Provisional Patent Application Ser. No. 63/288,468 filed on Dec. 10, 2021, the entire contents each of which are incorporated by reference herein.

### REFERENCE TO SEQUENCE LISTING

The Sequence Listing associated with this application is provided in XML format in lieu of a paper copy and is hereby incorporated by reference into the specification. The name of the XML file containing the Sequence Listing is 38T1382.xml. The text file is 65,612 bytes, was created on Jun. 5, 2024, and is being submitted electronically via Patent Center.

### FIELD OF THE DISCLOSURE

The present disclosure provides methods and compositions for priming a dimerizing agent regulated immunomodulatory complex for signaling by inducing the multimerization of at least a first fusion protein and a second fusion protein to thereby form the dimerizing agent regulated immunomodulatory complex. The methods and compositions utilize dimerizing agent dosing schedules designed to one or more of: (i) maintain specified blood trough levels of the dimerizing agent, (ii) allow activation of the immunomodulatory complex; (iii) reduce or avoid potential immunosuppressive effects of the dimerizing agent, (iv) reduce or avoid immune cell exhaustion, and/or (v) reduce or avoid side effects associated with activation of the immunomodulatory complex.

### BACKGROUND OF THE DISCLOSURE

Using genetic engineering, significant progress has been made in activating and directing cells of the immune system to kill cancer cells and infected cells. For example, T cells have been genetically engineered to express molecules having extracellular components that bind particular target antigens and intracellular components that direct actions of the T cell when the extracellular component has bound the target antigen. As an example, the extracellular component can be designed to bind target antigens found on cancer cells or infected cells and, when bound, the intracellular component activates the T cell to destroy the bound cell. Examples of such molecules include chimeric antigen receptors (CAR) for use in adoptive cellular immunotherapy (June et al., Nat. Biotechnol. 30:611, 2012; Restifo et al., Nat. Rev. Immunol. 12:269, 2012). Antigen binding stimulates the signaling domains on the intracellular segment of the CAR, thereby transducing signals that unleash inflammatory and cytotoxicity mechanisms. CAR-based adoptive cellular immunotherapy has been used to treat cancer patients with tumors refractory to conventional standard-of-care treatments (see Grupp et al., N. Engl. J. Med. 368:1509, 2013; Kalos et al., Sci. Transl. Med. 3:95ra73, 2011).

Despite the successes of CAR T cell therapies, safety and efficacy often remain a challenge. Safety challenges include cytokine release syndrome, neurotoxicity (Mirzaei, et al., Frontiers in immunology. 2017, 8, 1850; and Srivastava and Riddell, J Immunol. 2018, 200(2):459-468) and concern for aplasia or other toxicity due to expression of some antigen targets (e.g. CD33) on healthy tissue. Efficacy challenges include relapse due to antigen escape and T cell exhaustion (Gardner et al., Blood, 2016, 127(20): p. 2406-10; Ruella and Maus, Comput Struct Biotechnol J. 2016, 14:357-362; Haneen et al., Haematologica. 2018, 103(5):e215-e218). Next generation CAR T cell designs will need to address these concerns by providing a platform that can allow for controlled T cell activation.

### SUMMARY OF THE DISCLOSURE

The present disclosure utilizes methods and compositions for in vivo priming a dimerizing agent regulated immunomodulatory complex (DARIC) for signaling by inducing the multimerization of at least a first fusion protein and a second fusion protein to thereby form the DARIC. More particularly, the disclosure utilizes modulating the multimerization of a first fusion protein including a first multimerization domain (e.g., FKBP-rapamycin binding (FRB) or FK506 binding protein (FKBP)) and a second fusion protein including a second multimerization domain (e.g., FKBP-rapamycin binding (FRB) or FK506 binding protein (FKBP)) using rapamycin or analogs thereof for the formation of the DARIC.

In particular embodiments, the methods and compositions utilize dimerizing agent dosing schedules designed to one or more of: (i) maintain specified blood trough levels of the dimerizing agent, (ii) allow activation of the immunomodulatory complex; (iii) reduce or avoid potential immunosuppressive effects of the dimerizing agent, (iv) reduce or avoid immune cell exhaustion, and/or (v) reduce or avoid side effects associated with activation of the immunomodulatory complex.

In various embodiments, blood tough levels of the dimerizing agent are maintained within a range of 1-5 ng/mL. In particular embodiments, blood trough levels of the dimerizing agent are maintained within a range of 1.5-3 ng/mL. In various embodiments, a target blood tough level is 1 ng/mL, 1.5 ng/mL, 2 ng/mL, 2.5 ng/mL, 3 ng/mL, 3.5 ng/mL, 4 ng/mL, 4.5 ng/mL, or 5 ng/mL. In particular embodiments, a target trough blood level is 2 ng/mL.

In various embodiments, a subject is greater than 1.5 m² (body area of subject) and a dimerizing agent (e.g., rapamycin or analog thereof) is administered to the subject in need thereof at a dose range of 0.75-4 mg. In particular embodiments, a subject is greater than 1.5 m² and a dimerizing agent (e.g., rapamycin or analog thereof) is administered to the subject in need thereof at a dose of 0.75 mg, 1.0 mg, 1.25 mg, 1.5 mg, 1.75 mg, 2.0 mg, 2.25 mg, 2.5 mg, 2.75 mg, 3.0 mg, 3.25 mg, 3.5 mg, 3.75 mg, or 4 mg.

In various embodiments, a subject is less than or equal to 1.5 m² and a dimerizing agent is administered to the subject in need thereof at a dose of less than 0.75 mg/m². In particular embodiments, a subject is less than or equal to 1.5 m² and a dimerizing agent is administered to the subject in need thereof at a dose of 0.30 mg/m², 0.40 mg/m², 0.50 mg/m², 0.60 mg/m², or 0.70 mg/m².

In various embodiments, a dimerizing agent (e.g., rapamycin or analog thereof) is administered to a subject in need thereof daily, starting at least 16 hours after the subject has in vivo cells expressing DARIC. In particular embodiments, a dimerizing agent is administered to a subject in need thereof starting at day 1, 2, 3, 4, or 5 after the subject has in vivo cells expressing DARIC. In particular embodiments, a dimerizing agent is administered daily following the first administration of the dimerizing agent for 17, 18, 19, 20, 21, 22, 23, or 24 days. In particular embodiments, a dimerizing agent is administered to a subject in need thereof daily starting at day 2 after the subject has in vivo cells expressing DARIC and until day 21 after the subject has in vivo cells expressing DARIC.

In particular embodiments, after a first course of daily administration of a dimerizing agent, subject are not administered the dimerizing agent for a rest period. In certain examples, the rest period is 12 days, 13 days, 14 days, 15 days, or 16 days.

In particular embodiments, subjects who have had a rest period are administered a subsequent daily course of the dimerizing agent. The subsequent course generally will not begin less than 30, 31, 32, 33, 34, 35, 36, 37, 38, 39, 40, 41, 42, 43, 44, or 45 days from when the subject has in vivo cells expressing DARIC.

In particular embodiments, subjects demonstrating persistent disease are administered subsequent dimerizing agent courses. In particular embodiments, the disease includes leukemia. In particular embodiments, subjects in remission are administered subsequent dimerizing agent courses. In particular embodiments, subjects with an absence of Grade 3 or higher toxicity are administered subsequent dimerizing agent courses. In particular embodiments, subjects in remission with an absolute phagocyte count (APC) greater than 500 cells/μL are administered subsequent dimerizing agent courses. In particular embodiments, subsequent dimerizing agent courses are administered at day 42 after cells modified to express DARIC are infused into the subject. In particular embodiment, subsequent dimerizing agent courses are administered at 14 days after cessation of prior dimerizing agent administration.

### BRIEF DESCRIPTION OF THE FIGURES

Some of the drawings submitted herein may be better understood in color. Applicants consider the color versions of the drawings as part of the original submission and reserve the right to present color images of the drawings in later proceedings.

FIGS. 1A-1C. (1A) Lentiviral construct of CD33 VHH Dimerizing Agent Regulated Immunomodulatory Complex (DARIC33). DARIC33 separates antigen binding and signaling function of a chimeric antigen receptor (CAR). The lentiviral construct shown here incorporates use of a humanized camelid nanobody (VHH), for enhanced design modularity, targeting a novel epitope within the membrane proximal domain of CD33. Thus DARIC33 is a CD33 CAR with controllable, reversible activity, targeting a membrane proximal epitope within CD33. (1B) Mechanism of priming DARIC33 for signaling. Rapamycin (RAPA) addition heterodimerizes antigen binding and signaling chains through embedded FK506 binding protein (FKBP) and FKBP-rapamycin binding (FRB) rapamycin binding domains, reconstituting a immunomodulatory receptor complex poised for action. In the presence of target antigen, the 41bb and CD3z signaling domains activate T cell effector functions. Removing rapamycin returns DARIC33 to an inactive state where it can be reactivated following repeated addition of rapamycin. (1C) MV4-11 tumor progression following treatment of SC-DARIC33 or control T cells. Antitumor activity of SC-DARIC33 is compared with control T cells with and without rapamycin administration in vivo. Tumor cell lines were engineered to express a luciferase gene and the flux (photons/second) is graphed over a 36 day period.

FIG. 2. To benchmark the in vitro activity of DARIC33, dual CD19/CD33 expressing Raji cells were used as tumor xenografts. Following engraftment of dual expressing Raji cells in NOD Sicd gamma (NSG) mice, the mice were treated with either control mock T cells, CD19 CAR T cells or DARIC33 T cells with or without rapamycin.

FIG. 3. Mice were treated with CD33⁺ MV4-11 AML cells at day −7 and SC-DARIC33 was administered on Day 1. Graph shows the flux (photon/second) of mock T cells, rapamycin alone, 1×10⁷ DARIC 33 cells, and 1×10⁷ DARIC 33 cells with rapamycin for the days following the tumor injection.

FIG. 4A-4F. Activation of SC-DARIC33 is reversible. (4A) DARIC33 cell cytokine responses to antigen at various times following wash out from rapamycin containing media. DARIC33 cells replaced into rapamycin containing media or DARIC33 cells previously cultured in media not containing rapamycin were used as comparators. The t_{1/2} is determined by fitting a single-phase exponential decay. (4B) 10⁷ SC-DARIC33+ cells, or an equivalent number of UTD control cells were infused intravenously (IV) in NSG mice 7 days after engraftment of 1×10⁶ MV4-11.ff/luc leukemia cells. Following T cell infusion, mice were treated with 0.1 mg/kg rapamycin 3 times weekly for the indicated durations or were observed. (4C) Tumor progression monitored by bioluminescence, n=5 mice per group. Images taken during a ‘pause’ in rapamycin dosing are outlined (5^{TH} column, rows 4-13 and 6^{th} column, rows 4 and 5). (4D) Quantitation of tumor growth. Points are measurements of individual mice, best-fit tumor growth trajectories. (4E) Tumor growth rates. Points are growth rates fit for individual mice, box and whiskers show mean and standard deviation, asterisks indicate ** p<0.01, t-tests, with Benjamini-Hochberg correction for multiple comparisons. (4F) Survival after infusion of DARIC33 cells or UTD cells following by treatment with various rapamycin schedules. Mantel-Cox log-rank p values are shown uncorrected.

FIG. 5A-5E. In vitro modeling of SC-DARIC33 rapamycin response allows targeted rapamycin dosing in vivo. (5A) Cytokine release following stimulation of DARIC33 cells with MV4-11 AML cells in media or whole blood in the presence of increasing rapamycin concentrations. IFN^{γ} responses are normalized per donor and apparent EC50s determined using a four-parameter logistic dose response curves are reported. (5B) Determination of rapamycin pharmacokinetics in mice. Concentrations of rapamycin in whole blood obtained during administration of various rapamycin doses 3 times weekly are shown above, along with the timing of IP rapamycin injections, bars, below. Upper limit of quantitation (ULOQ=200 ng/mL) and lower limit of quantitation (LLOQ=1 ng/mL) are indicated. (5C, 5D) AML tumor progression in mice following treatment with DARIC33 and various dose schedules of rapamycin days 0-18 post T cell infusion. (5C) Schematic illustrating experimental design. (5D) Quantitation of tumor growth kinetics. Points represent bioluminescence measures of individual mice (n=5-10 per group) and lines indicate tumor growth trajectories modeled using linear mixed effects. (5E) tumor progression of control and Kaplan Meier survival plots.

FIG. 6. Whole Blood Rapamycin Concentration in Xenografted and Treated Mice.

FIG. 7. Whole Blood Rapamycin vs Time in Pediatric Patients. Rapamycin doses in pediatric patients predicted to prime SC-DARIC33 for signaling were estimated by integrating peds pt PK data from a population model; typical immunosuppressive exposures; whole blood rapamycin EC50 in vitro for SC-DARIC33; and rapamycin associated with efficacy in mouse xenografts. The recommended rapamycin starting daily dose is 0.50 mg/m² (for patients≤1.5 m²) or up to 4.0 mg (for patients>1.5 m²). In some embodiments, the starting daily dose is 0.75 mg or 1.5 mg depending on the age of the patients. Patients included pediatric patients, adult patients and pediatric-adult (e.g., 18-28 years) patients. The predicted rapamycin concentration in whole blood trough result is 1.5-3 ng/mL in most patients.

FIGS. 8A and 8B. Protocols for administering DARIC33 and Rapamycin to patients with lymphodepletion. Trial Schema (8A) shows that apheresis, SC-DARIC33 T cell manufacture, and bridging therapy are performed before lymphodepletion at day −2. At day 0, subjects are infused with SC-DARIC33. At day 2, rapamycin is administered until day 21 post infusion. At this time, rapamycin administration can stop. At day 42, rapamycin administration can either resume or not depending on the subject's response to the treatment. Rapamycin can be administered in cycles. During the course of this administration protocol, bone marrow aspirates and/or biopsies are taken at various timepoints such as t day −5, day 14, day 28, and/or day 42. (8B) shows an alternative protocol wherein pediatric patients with relapsed or refractory AML receive escalating cell doses of DARIC33 following lymphodepleting chemotherapy with fludarabine and cyclophosphamide. Rapamycin is administered on days 3-21.

FIG. 9. Several Sirolimus dose levels (0.5 mg, 0.75 mg, 1.0 mg, 1.25 mg and 1.5 mg) were simulated on a once daily dosing schedule for 19-21 days.

FIG. 10. Exposure profiles for a starting dose of 1.5 mg daily were generated showing geometric mean (solid line) and 10^{th}, 90^{th} percentiles (shading) of expected Sirolimus concentrations. An initial dose of 1.5 mg daily will enable a significant fraction of patients to attain target sirolimus concentrations of 1.5-3 ng/mL. Dosing adjustments can be made.

FIG. 11. Patient (>1.5 m²) pharmacokinetic (PK) data following rapamycin administration demonstrating the exposure relationship between dose and peak and trough levels, and dose adjustments to achieve the target range. Rapamycin was initiated orally at a dose of 0.75 mg and peak and trough levels were monitored as described using the clinical LC-MS/MS assay after each dose.

### DETAILED DESCRIPTION

Using genetic engineering, significant progress has been made in activating and directing cells of the immune system to kill cancer cells and infected cells. For example, T cells have been genetically engineered to express molecules having extracellular components that bind particular target antigens and intracellular components that direct actions of the T cell when the extracellular component has bound the target antigen. As an example, the extracellular component can be designed to bind target antigens found on cancer cells or infected cells and, when bound, the intracellular component activates the T cell to destroy the bound cell. Examples of such molecules include chimeric antigen receptors (CAR) for use in adoptive cellular immunotherapy (June et al., Nat. Biotechnol. 30:611, 2012; Restifo et al., Nat. Rev. Immunol. 12:269, 2012). Antigen binding stimulates the signaling domains on the intracellular segment of the CAR, thereby transducing signals that unleash inflammatory and cytotoxicity mechanisms. CAR-based adoptive cellular immunotherapy has been used to treat cancer patients (or subjects) with tumors refractory to conventional standard-of-care treatments (see Grupp et al., N. Engl. J. Med. 368:1509, 2013; Kalos et al., Sci. Transl. Med. 3:95ra73, 2011).

Despite the successes of CAR T cell therapies, safety and efficacy often remain a challenge. Safety challenges include cytokine release syndrome, neurotoxicity (Mirzaei, et al., Frontiers in immunology. 2017, 8, 1850; and Srivastava and Riddell, J Immunol. 2018, 200(2):459-468) and concern for aplasia due to expression of some antigen targets (e.g. CD33) on healthy tissue. Efficacy challenges include relapse due to antigen escape and T cell exhaustion (Gardner et al., Blood, 2016, 127(20): p. 2406-10; Ruella and Maus, Comput Struct Biotechnol J. 2016, 14:357-362; Haneen et al., Haematologica. 2018, 103(5):e215-e218).

This disclosure addresses these concerns by providing a platform that can allow for controlled immune cell activation. By obtaining control of the activity of engineered immunomodulatory molecules (e.g., engineered receptors, such as CARs), advantages including improved toxicity profile as well as prevention of exhaustion. This ability to control activity of the engineered immunomodulatory molecules may be of added importance when targeting myeloid antigens where there is concern for marrow aplasia. The present disclosure provides methods and compositions for priming a dimerizing agent regulated immunomodulatory complex (DARIC) for signaling by inducing the multimerization of at least a first fusion protein and a second fusion protein to thereby form the DARIC. More particularly, the disclosure relates to modulating the multimerization of a first fusion protein including an FKBP-rapamycin binding multimerization domain and a second fusion protein including an FK506 binding protein multimerization domain using rapamycin or analogs thereof for the formation of a DARIC that is primed for signaling.

In particular embodiments, a subject in need of treatment thereof is administered a dose of cells that express a DARIC, wherein the DARIC includes a first fusion protein including a first multimerization domain and a second fusion protein including a second multimerization domain; and administering a dimerizing agent that binds the first and second multimerization domains.

In particular embodiments, a subject in need of treatment thereof is administered a dose of cells that express a DARIC, wherein the DARIC includes a first fusion protein including a FKBP-rapamycin binding (FRB) multimerization domain and a second fusion protein including a FK506 binding protein (FKBP) multimerization domain; and administering rapamycin or an analog thereof that binds the multimerization domains.

In particular embodiments, a subject in need of treatment thereof is administered a dose of cells that express a DARIC, wherein the DARIC includes a first fusion protein including a FK506 binding protein (FKBP) multimerization domain and a second fusion protein including a FKBP-rapamycin binding (FRB) multimerization domain; and administering rapamycin or an analog thereof that binds the multimerization domains.

In particular embodiments, a subject in need of treatment thereof is administered a composition, wherein the compositions edits cells of the subject to express a DARIC, wherein the DARIC includes a first fusion protein including a first multimerization domain and a second fusion protein including a second multimerization domain; and administering a dimerizing agent that binds the first and second multimerization domains.

In particular embodiments, a subject in need of treatment thereof is administered a composition, wherein the compositions edits cells of the subject to express a DARIC, wherein the DARIC includes a first fusion protein including a FKBP-rapamycin binding (FRB) multimerization domain and a second fusion protein including a FK506 binding protein (FKBP) multimerization domain; and administering a dimerizing agent that binds the multimerization domains.

In particular embodiments, a subject in need of treatment thereof is administered a composition, wherein the compositions edits cells of the subject to express a DARIC, wherein the DARIC includes a first fusion protein including a FK506 binding protein (FKBP) multimerization domain and a second fusion protein including a FKBP-rapamycin binding (FRB) multimerization domain; and administering a dimerizing agent that binds the multimerization domains.

In particular embodiments, a DARIC is primed for signaling by administering a dimerizing agent. In particular embodiments, the dimerizing agent is rapamycin. In particular embodiments, the dimerizing agent is a rapamycin analog.

In particular embodiments, the DARIC includes a first fusion protein including a first multimerization domain and a second fusion protein including a second multimerization domain. In particular embodiments, the first fusion protein includes a binding domain, a first transmembrane domain, and a first multimerization domain; and the second fusion protein includes a second multimerization domain, a second transmembrane domain, and an intracellular component. In particular embodiments, the first fusion protein includes a first transmembrane domain, and a first multimerization domain, and first intracellular signaling component; and the second fusion protein includes a second multimerization domain, a second transmembrane domain, and a second intracellular component. In particular embodiments, the multimerization domains localize extracellularly when the fusion proteins are expressed. In particular embodiments, the multimerization domains localize intracellularly when the fusion proteins are expressed.

In particular embodiments, the first multimerization domain is an FRB multimerization domain and the second multimerization domain is an FKBP multimerization domain. In particular embodiments, the first multimerization domain is an FKBP multimerization domain and the second multimerization domain is an FRB multimerization domain. In particular embodiments, the binding domain includes an anti-CD33 VHH antibody and/or an anti-CLL1 VHH antibody. In particular embodiments, the first transmembrane domain includes a CD4 transmembrane domain or a CD8α transmembrane domain. In particular embodiments, the second transmembrane domain includes a CD4 transmembrane domain or a CD8α transmembrane domain. In particular embodiments, an intracellular component includes a CD3ζ primary intracellular signaling domain. In particular embodiments, an intracellular component includes a 4-1 BB costimulatory domain. In particular embodiments, an intracellular component includes an OX40 or TNFR2 costimulatory domain.

In particular embodiments, a subject is greater than 1.5 m² and a dimerizing agent is administered to the subject in need thereof at a dose of 0.75 mg, 1.0 mg, 1.25 mg, 1.5 mg, 1.75 mg, 2.0 mg, 2.25 mg, 2.5 mg, 2.75 mg, 3.0 mg, 3.25 mg, 3.5 mg, 3.75 mg, or 4 mg. In particular embodiments, a subject is less than or equal to 1.5 m² and a dimerizing agent is administered to the subject in need thereof at a dose of less than or equal to 0.75 mg/m². In particular embodiments, a subject is less than or equal to 1.5 m² and a dimerizing agent is administered to the subject in need thereof at a dose of 0.30 mg/m², 0.40 mg/m², 0.50 mg/m², 0.60 mg/m², or 0.70 mg/m². In particular embodiments, a subject is less than or equal to 1.5 m² and a dimerizing agent is administered to the subject in need thereof at a dose of 0.50 mg/m².

In particular embodiments, a dimerizing agent is administered to a subject in need thereof at a dose, wherein the dose maintains a target trough blood level ranging from 1.5-3 ng/mL. In particular embodiments, a dimerizing agent is administered to a subject in need thereof at a dose, wherein the dose maintains a target trough blood level ranging from 1-4.5 ng/mL, 1-4 ng/mL, 1-3.5 ng/mL, and 1-3 ng/mL, 1.5-5 ng/mL, 1.5-4.5 ng/mL, 1.5-4 ng/mL, 1.5-3.5 ng/mL, or 1.5-3 ng/mL. In particular embodiments, a dimerizing agent is administered to a subject in need thereof at a dose, wherein the dose maintains a target trough blood level of 2 ng/mL.

In particular embodiments, a subject is greater than 1.5 m² and a rapamycin or an analog thereof is administered to the subject in need thereof at a dose of 0.75 mg. In particular embodiments, a subject is less than or equal to 1.5 m² and a rapamycin or an analog thereof is administered to the subject in need thereof at a dose of 0.50 mg/m². In particular embodiments, a rapamycin or an analog thereof is administered to a subject in need thereof at a dose, wherein the dose maintains a target trough blood level of 2 ng/mL. In particular embodiments, a rapamycin or an analog thereof is administered to a subject in need thereof at a dose, wherein the dose maintains a target trough blood level ranging from 1.5-3 ng/mL.

In particular embodiments, a dimerizing agent (e.g., rapamycin or analog thereof) is administered to a subject in need thereof daily, starting at least 16 hours after the subject has in vivo cells expressing DARIC. In particular embodiments, a dimerizing agent is administered to a subject in need thereof starting at day 1, 2, 3, 4, or 5 after the subject has in vivo cells expressing DARIC. In particular embodiments, a dimerizing agent is administered daily following the first administration of the dimerizing agent for 17, 18, 19, 20, 21, 22, 23, or 24 days. In particular embodiments, a dimerizing agent is administered to a subject in need thereof daily starting at day 2 after the subject has in vivo cells expressing DARIC and until day 21 after the subject has in vivo cells expressing DARIC. In particular embodiments, a dimerizing agent is administered to a subject in need thereof daily starting at day 3 after the subject has in vivo cells expressing DARIC and until day 21 after the subject has in vivo cells expressing DARIC.

In particular embodiments, after a first course of daily administration of a dimerizing agent, subject is not administered the dimerizing agent for a rest period. In certain examples, the rest period is 12 days, 13 days, 14 days, 15 days, or 16 days. In certain examples, the rest period is at least 12 days, at least 13 days, at least 14 days, at least 15 days, or at least 16 days. In certain examples, the rest period is no more than 21 days. In certain examples, the rest period is no more than 28 days. In certain examples, the rest period is no more than 1 month, 2 months, or 3 months.

In particular embodiments, subjects who have had a rest period are administered a subsequent daily course of the dimerizing agent. The subsequent course generally will not begin less than 36, 37, 38, 39, 40, 41, 42, 43, 44, or 45 days from when the subject has in vivo cells expressing DARIC.

In particular embodiments, subjects demonstrating persistent disease are administered subsequent dimerizing agent courses. In particular embodiments, disease includes leukemia. In particular embodiments, subjects in remission are administered subsequent dimerizing agent courses. In particular embodiments, subjects with an absence of Grade 3 or higher toxicity are administered subsequent dimerizing agent courses. In particular embodiments, subjects in remission with an absolute phagocyte count (APC) greater than 500 cells/μL are administered subsequent dimerizing agent courses. In particular embodiment, subsequent dimerizing agent courses are administered at day 42 after cells modified to express DARIC are infused into subject. In particular embodiment, subsequent dimerizing agent courses are administered at 14 days after cessation of prior dimerizing agent administration.

In particular embodiments, subjects demonstrating persistent disease are administered subsequent courses of rapamycin or analogs thereof. In particular embodiments, disease includes leukemia. In particular embodiments, subjects in remission are administered subsequent courses of rapamycin or analogs thereof. In particular embodiments, subjects with an absence of Grade 3 or higher toxicity are administered subsequent courses of rapamycin or analogs thereof. In particular embodiments, subjects in remission with an absolute phagocyte count (APC) greater than 500 cells/μL are administered subsequent dimerizing agent courses. In particular embodiment, subsequent courses of rapamycin or analogs thereof are administered at day 42 after cells modified to express DARIC are infused into subject. In particular embodiment, subsequent courses of rapamycin or analogs thereof are administered at 14 days after cessation of prior dimerizing agent administration.

Aspects of the current disclosure are now described with additional detail and options as follows: (i) Dimerizing Agent Regulated Immunomodulatory Complexes (DARIC); (ii) Dimerizing Agents; (iii) Fusion Proteins; (iii-a) Multimerization Domains; (iii-b) Binding Domains; (iii-c) Intracellular Components; (iii-d) Transmembrane Domains; (iii-e) Linkers; (iii-f) Tags and Selectable Markers; (iv) Polynucleotides; (v) Genetically Modified Cells; (vi) Formulations; (vii) Therapeutic Methods; (viii) Exemplary Embodiments; (ix) Examples; (x) Sequences Supporting the Disclosure; and (xi) Closing Paragraphs. These headings are provided for organizational purposes only and do not limit the scope or interpretation of the disclosure.

### (i) DIMERIZING AGENT REGULATED IMMUNOMODULATORY COMPLEXES (DARIC)

The present disclosure provides methods and compositions for multimerizing components of a DARIC such that the DARIC is primed for signaling. In particular embodiments, a DARIC includes a first fusion protein including a first multimerization domain and a second fusion protein including a second multimerization domain and an intracellular component, wherein a dimerizing agent binds the first and second multimerization domains such that the first and second fusion proteins multimerize to form a DARIC ready for activation (i.e., primed for signaling).

The temporal control achieved through the multimerization mechanism described herein only primes the machinery for signaling. Unlike conventional CARs or other engineered receptors which are already primed for activation, DARIC require administration of a dimerizing agent to be primed for activation. In particular embodiments, the dimerizing agent induced multimerization reconstitutes a signaling-potentiated receptor, but it does not activate downstream signaling because there is no aggregation of intracellular signaling components. Spatial control is, therefore, achieved on the basis of the presence or absence of a target recognized by the binding domain of one of the fusion proteins. Since the binding domain of the fusion protein is secreted to the outside of the cell (or applied extraneously), it accumulates only where target is present, such that cells will only become activated when both target (e.g., cell surface antigen) and the dimerizing agent are present. In particular embodiments, the multimerization domains localize extracellularly when the fusion proteins are expressed. In particular embodiments, the multimerization domains localize intracellularly when the fusion proteins are expressed.

In particular embodiments, “primed for signaling”, “priming for signaling”, “primes for signaling”, and similar phrases thereof (e.g., “priming a DARIC for signaling”) refers to the reconfiguration of the components of the DARIC such that a fusion protein including the binding domain and a fusion protein including the intracellular component are functionally coupled such that activation or downstream signaling can occur within the engineered cell upon binding a target antigen. In some places herein, a DARIC is referred to as activated or active when it is primed for signaling. In some embodiments, a DARIC does not include a binding domain. In some embodiments, a DARIC not including a binding domain includes an intracellular component on each of the first and second fusion proteins, wherein signaling occurs upon multimerization.

Herein, “engineered” refers to a cell, microorganism, organism, nucleic acid molecule, or vector that has been genetically altered or modified by introduction of a heterologous nucleic acid molecule, or refers to a cell that has been altered such that the expression of an endogenous nucleic acid molecule or gene can be controlled.

As used herein, “heterologous” nucleic acid molecule, construct or sequence refers to a nucleic acid molecule or portion of a nucleic acid molecule sequence that is not native to a cell in which it is expressed, a nucleic acid molecule or portion of a nucleic acid molecule native to a host cell that has been altered or mutated, or a nucleic acid molecule with an altered expression as compared to the native expression levels under similar conditions.

In particular embodiments, a DARIC can include a dimer, trimer, or higher order multimer formed by at least two different proteins, including at least one protein having a binding domain specific for a target and/or one protein having an intracellular component, such as an intracellular signaling domain, a co-stimulatory domain, or a co-receptor domain. The DARIC is primed for signaling when a dimerizing agent(s) brings together at least two of the proteins and the associated proteins together. In certain embodiments, the DARIC includes at least a an intracellular component that allows transmission of or transmits an intracellular signal. In other embodiments, the DARIC includes a binding domain.

### (ii) DIMERIZING AGENT

A “dimerizing agent” refers to any molecule capable of binding to a first multimerization domain and second multimerization domain, thus bringing together the two multimerization domains and any constituents thereby attached to the multimerization domain.

In particular embodiments, the dimerizing agent is rapamycin (sold under the brand name Rapamune® (Amgen, Thousand Oaks, CA) and also known as sirolimus). Rapamycin analogs (rapalogs) can also be used. Exemplary rapamycin analogs include those disclosed in U.S. Pat. No. 6,649,595, which describes various rapalog structures. In certain embodiments, a dimerizing agent is a rapalog with substantially reduced immunosuppressive effect as compared to rapamycin. A “substantially reduced immunosuppressive effect” refers to a rapalog having at least less than 0.1 to 0.005 times the immunosuppressive effect observed or expected for an equimolar amount of rapamycin, as measured either clinically or in an appropriate in vitro (e.g., inhibition of T cell proliferation) or in vivo surrogate of human immunosuppressive activity. Alternatively, “substantially reduced immunosuppressive effect” refers to a rapalog having an EC₅₀ value in such an in vitro assay that is at least 10 to 250 times larger than the EC₅₀ value observed for rapamycin in the same assay. Other exemplary rapalogs include everolimus, novolimus, pimecrolimus, ridaforolimus, tacrolimus, temsirolimus, umirolimus, zotarolimus, rimiducid (AP1903), AP20187 (other names: 2,2′-[[2-[(dimethylamino)methyl]-1,3-propanediyl]bis[imino(2-oxo-2,1-ethanediyl)oxy-3,1-phenylene[(1R)-3-(3,4-dimethoxyphenyl)propylidene]]]ester; (2S,2'S)-1-[(2S)-1-oxo-2-(3,4,5-trimethoxyphenyl)butyl]-2-piperidinecarboxylic acid B/B Homodimerizer), AP21967 (other names: C16-(S)-7-methylindolerapamycin; C16-AiRap) and BPC015.

In particular embodiments, dimerizing agents include rapamycin (sirolimus) or a rapalog thereof, coumermycin or a derivative thereof, gibberellin or a derivative thereof, abscisic acid (ABA) or a derivative thereof, methotrexate or a derivative thereof, cyclosporin A or a derivative thereof, FKCsA or a derivative thereof, trimethoprim (Tmp)-synthetic ligand for FKBP (SLF) or a derivative thereof, or any combination thereof.

In other particular embodiments, an anti-dimerizing agent blocks the association of at least the two first fusion proteins with the dimerizing agent. For example, cyclosporin or FK506 could be used as anti-dimerizing agents to titrate out the dimerizing agent and, therefore, stop signaling since only one multimerization domain is bound. In certain embodiments, an anti-dimerizing agent (e.g., cyclosporine, FK506) is an immunosuppressive agent. For example, an immunosuppressive anti-dimerizing agent may be used to block or minimize the function of the fusion proteins of the instant disclosure and at the same time inhibit or block an unwanted or pathological inflammatory response in a clinical setting.

### (iii) FUSION PROTEINS

A “fusion protein” refers to a protein that includes polypeptide components derived from one or more parental proteins or polypeptides (e.g., fusion polypeptides) and does not naturally occur in a host cell. A fusion protein will contain two or more naturally-occurring amino acid sequences that are linked together in a way that does not occur naturally. For example, a fusion protein may have two or more portions from the same protein linked in a way not normally found in a cell, or a fusion protein may have portions from two, three, four, five or more different proteins linked in a way not normally found in a cell. A fusion protein can be encoded by a nucleic acid molecule wherein a nucleotide sequence encoding one protein or portion thereof is appended in frame with, and optionally separated by nucleotides that encode a linker, spacer or junction amino acids, a nucleic acid molecule that encodes one or more different proteins or a portion thereof. In certain embodiments, a nucleic acid molecule encoding a fusion protein is introduced into a host cell and expressed.

As used herein, the term “host” refers to a cell (e.g., T cell) or microorganism that may be genetically modified with an exogenous nucleic acid molecule to produce a polypeptide of interest (e.g., DARIC binding or signaling components). In certain embodiments, a host cell may optionally already possess or be modified to include other genetic modifications that confer desired properties related or unrelated to fusion protein biosynthesis (e.g., deleted, altered or truncated TCR, checkpoint protein, or other gene; increased costimulatory factor expression). In certain embodiments, a host cell is a human T cell or a human T cell with TCRα, TCRβ, or both knocked out with a site-specific nuclease (e.g., a LAGLIDADG homing endonuclease, LHE).

In particular embodiments, a DARIC includes a first fusion protein including a first multimerization domain and a second fusion protein including a second multimerization domain. In particular embodiments, the first fusion protein includes a binding domain, a first transmembrane domain, and a first multimerization domain; and the second fusion protein includes a second multimerization domain, a second transmembrane domain, and an intracellular component. In particular embodiments, a first and/or second fusion protein can optionally include linkers, tags, or selectable markers.

A fusion protein of a fusion protein can contain more than one multimerization domain, including a multimerization domain that promotes homodimerization in the presence of homo-bivalent dimerizing agent. In such embodiments, the administration of a dimerizing agent will promote some level of basal signaling in the absence of binding to an extracellular target—for example, as a way to drive cell proliferation in vitro or in vivo prior to activation. For T cells, it is known that lower-level activation promotes proliferation, whereas the higher order multimerization (as would occur by high density of antigen on a target cell and heterodimerization of the fusion proteins with dimerizing agents) would lead to activation of a cytotoxicity response.

In certain embodiments, a fusion protein can have multiple binding domains. For example, an engineered cell can express a third fusion protein including a binding domain and a second multimerization domain, optionally a transmembrane domain or a transmembrane domain with intracellular component, wherein the third fusion protein localizes extracellularly when expressed. In related embodiments, the fusion proteins include one, two, three, or four binding domains, wherein the one, two, three, or four binding domains are specific for one target or up to four different targets. In particular embodiments, a DARIC includes a binding domain for CD33 and CLL1.

Fusion proteins can include one or more polypeptide domains or segments including signal peptides, cell permeable peptide domains (CPP), binding domains, signaling domains, etc., epitope tags (e.g., maltose binding protein (“MBP”), glutathione S transferase (GST), HIS6, MYC, FLAG, V5, VSV-G, and HA), polypeptide linkers, and polypeptide cleavage signals. Fusion proteins and polypeptides are typically linked C-terminus to N-terminus, although they can also be linked C-terminus to C-terminus, N-terminus to N-terminus, or N-terminus to C-terminus. In particular embodiments, the polypeptides of the fusion protein can be in any order. Fusion polypeptides or fusion proteins can also include conservatively modified variants, polymorphic variants, alleles, mutants, subsequences, and interspecies homologs, so long as the desired activity of the fusion polypeptide is preserved. Fusion polypeptides may be produced by chemical synthetic methods or by chemical linkage between the two moieties or may generally be prepared using other standard techniques. Ligated DNA sequences including the fusion polypeptide are operably linked to suitable transcriptional or translational control elements as disclosed elsewhere herein.

Fusion proteins and polypeptides may optionally include one or more linkers that can be used to link the one or more polypeptides or domains within a polypeptide. A peptide linker sequence may be employed to separate any two or more polypeptide components by a distance sufficient to ensure that each polypeptide folds into its appropriate secondary and tertiary structures so as to allow the polypeptide domains to exert their desired functions. Such a peptide linker sequence is incorporated into the fusion polypeptide using standard techniques in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. In particular embodiments, preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. Linker sequences are not required when a particular fusion polypeptide segment contains non-essential N-terminal amino acid regions that can be used to separate the functional domains and prevent steric interference. In particular embodiments, preferred linkers are typically flexible amino acid subsequences which are synthesized as part of a recombinant fusion protein. Linker polypeptides can be between 1 and 200 amino acids in length, between 1 and 100 amino acids in length, or between 1 and 50 amino acids in length, including all integer values in between.

Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase.

In particular embodiments, the polypeptide cleavage signal is a viral self-cleaving peptide or ribosomal skipping sequence.

Illustrative examples of ribosomal skipping sequences include: a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide.

In one embodiment, the viral 2A peptide is selected from the group including: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

(iii-a) Multimerization Domain.

A “multimerization domain,” as used herein, refers to a molecule that preferentially interacts or associates with another molecule directly or via a dimerizing agent, wherein the interaction of the different multimerization domains substantially contribute to or efficiently promote multimerization (i.e., the formation of a dimer, trimer, or multipartite complex, which may be a homodimer, heterodimer, homotrimer, heterotrimer, homomultimer, heteromultimer).

In particular embodiments, multimerization domains will associate using a dimerizing agent. In particular embodiments, the dimerizing agent is rapamycin or an analog thereof. For example, the first and second multimerization domains are a pair selected from a FK506 binding protein (FKBP) multimerization domain and a FKBP-rapamycin binding (FRB) multimerization domain, or variants thereof. FRB domains are polypeptide regions (protein “domains”) that are capable of forming a tripartite complex with an FKBP protein and rapamycin or rapalog thereof. FRB domains are present in a number of naturally occurring proteins, including mTOR proteins (also referred to in the literature as FRAP, RAPT1, or RAFT) from human and other species; yeast proteins including Tor1 and Tor2; and a Candida FRAP homolog. Information concerning the nucleotide sequences, cloning, and other aspects of these proteins is known in the art. For example, a protein sequence accession number for a human mTOR is GenBank Accession No. L34075.1 (Brown et al., Nature 369:756, 1994).

In particular embodiments, the first and second multimerization domains localize extracellularly when the first and second fusion proteins are expressed. In particular embodiments, the first and second multimerization domains localize intracellularly when the first and second fusion proteins are expressed.

In particular embodiments, the term “FKBP-rapamycin binding (FRB) multimerization domain” refers to an FRB polypeptide. FRB domains for use in the fusion proteins of this disclosure generally contain at least 85 to 100 amino acid residues. In certain embodiments, an FRB amino acid sequence for use in fusion proteins of this disclosure will include a 93 amino acid sequence Ile-2021 through Lys-2113 and a mutation of T2098L (T82L is equivalent position in 93 amino acid FRB polypeptide), with reference to GenBank Accession No. L34075.1. An FRB domain for use in fusion proteins of this disclosure will be capable of binding to a complex of an FKBP protein bound to rapamycin or an analog thereof of this disclosure. In certain embodiments, a peptide sequence of an FRB domain includes (a) a naturally occurring peptide sequence spanning at least the indicated 93 amino acid region of human mTOR or corresponding regions of homologous proteins; (b) a variant of a naturally occurring FRB in which up to ten amino acids, or 1 to 5 amino acids or 1 to 3 amino acids, or in some embodiments just one amino acid, of the naturally-occurring peptide have been deleted, inserted, or substituted; or (c) a peptide encoded by a nucleic acid molecule capable of selectively hybridizing to a DNA molecule encoding a naturally occurring FRB domain or by a DNA sequence which would be capable, but for the degeneracy of the genetic code, of selectively hybridizing to a DNA molecule encoding a naturally occurring FRB domain. In particular embodiments, an FRB polypeptide binds to an FKBP polypeptide through a bridging factor, thereby forming a ternary complex.

Particular embodiments utilize the FRB sequence: ILWHEMWHEGLEEASRLYFGERNVKGMFEVLEPLHAMMERGPQTLKETSFNQAYGRDLMEA QEWCRKYMKSGNVKDLLQAWDLYYHVFRRISK (SEQ ID NO: 55) and particular embodiments utilize the sequence:

SEQ ID NO: 56)
ILWHEMWHEGLEEASRLYFGERNVKGMFEVLEPLHAMMERGPQTLKETS
FNQAYGRDLMEAQEWCRKYMKSGNVKDLIQAWDLYYHVFRRISK.

In particular embodiments, the term “FK506 binding protein (FKBP) multimerization domain” refers to an FKBP polypeptide. FKBPs are the cytosolic receptors for macrolides, such as FK506, FK520 and rapamycin, and are highly conserved across species lines. For the purpose of this disclosure, FKBPs are proteins or protein domains that are capable of binding to rapamycin or to an analog thereof and further forming a tripartite complex with an FRB-containing protein or fusion protein. An FKBP domain may also be referred to as a “rapamycin binding domain”. Information concerning the nucleotide sequences, cloning, and other aspects of various FKBP species is known in the art (see, e.g., Staendart et al., Nature 346:671, 1990 (human FKBP12); Kay, Biochem. J. 314:361, 1996). Homologous FKBP proteins in other mammalian species, in yeast, and in other organisms are also known in the art and may be used in the fusion proteins disclosed herein. The size of FKBP domains for use in the disclosure varies, depending on which FKBP protein is employed. An FKBP domain of a fusion protein of this disclosure will be capable of binding to rapamycin or an analog thereof and participating in a tripartite complex with an FRB-containing protein (as may be determined by any means, direct or indirect, for detecting such binding).

The peptide sequence of an FKBP domain of an FKBP fusion protein of the disclosure includes (a) a naturally occurring FKBP peptide sequence, preferably derived from the human FKBP12 protein (GenBank Accession No. AAA58476.1) or a peptide sequence derived therefrom, from another human FKBP, from a murine or other mammalian FKBP, or from some other animal, yeast or fungal FKBP; (b) a variant of a naturally occurring FKBP sequence in which up to ten amino acids, or 1 to 5 amino acids or 1 to 3 amino acids, or in some embodiments just one amino acid, of the naturally-occurring peptide have been deleted, inserted, or substituted; or (c) a peptide sequence encoded by a nucleic acid molecule capable of selectively hybridizing to a DNA molecule encoding a naturally occurring FKBP or by a DNA sequence which would be capable, but for the degeneracy of the genetic code, of selectively hybridizing to a DNA molecule encoding a naturally occurring FKBP. In particular embodiments, the FKBP polypeptide is an FKBP12 polypeptide or an FKBP12 polypeptide including an F36V mutation. In particular embodiments, an FKBP polypeptide contemplated herein binds to an FRB polypeptide through a bridging factor, thereby forming a ternary complex.

Particular embodiments utilize the sequence:

(SEQ ID NO: 57)
GVQVETISPGDGRTFPKRGQTCVVHYTGMLEDGKKFDSSRDRNKPFKFM
LGKQEVIRGWEEGVAQMSVGQRAKLTISPDYAYGATGHPGIIPPHATLV
FDVELLKLE.

And particular embodiments utilize the sequence:

(SEQ ID NO: 58)
GVQVETISPGDGRTFPKRGQTCVVHYTGMLEDGKKVDSSRDRNKPFKFM
LGKQEVIRGWEEGVAQMSVGQRAKLTISPDYAYGATGHPGIIPPHATLV
FDVELLKLE.

A “bridging factor” refers to a molecule that associates with and that is disposed between two or more multimerization domains. In particular embodiments, multimerization domains substantially contribute to or efficiently promote formation of a polypeptide complex only in the presence of a bridging factor. In particular embodiments, multimerization domains do not contribute to or do not efficiently promote formation of a polypeptide complex in the absence of a bridging factor. Illustrative examples of bridging factors suitable for use in particular embodiments contemplated herein include AP21967, rapamycin (sirolimus) or a rapalog thereof, coumermycin or a derivative thereof, gibberellin or a derivative thereof, abscisic acid (ABA) or a derivative thereof, methotrexate or a derivative thereof, cyclosporin A or a derivative thereof, FKCsA or a derivative thereof, trimethoprim (Tmp)-synthetic ligand for FKBP (SLF) or a derivative thereof, or any combination thereof.

Other multimerization domain pairs include FKBP and calcineurin, FKBP and cyclophilin, FKBP and bacterial DHFR, calcineurin and cyclophilin, PYL1 and AB11, or GIB1 and GAI, or variants thereof.

In particular embodiments, the first multimerization domain is an FRB multimerization domain and the second multimerization domain is an FKBP multimerization domain. In particular embodiments, the first multimerization domain is an FKBP multimerization domain and the second multimerization domain is an FRB multimerization domain. In particular embodiments, the dimerizing agent/bridging factor is a rapamycin and/or analog thereof.

In certain embodiments, the first and second multimerization domains are the same or different.

(iii-b) Binding Domains.

A “binding domain” refers to a protein, polypeptide, oligopeptide, peptide or other molecule that possesses the ability to specifically recognize and bind to a target (e.g., CD19, CD20, CD33, CLL1 and/or other target antigen).

Fusion protein binding domains useful in the instant disclosure include those known in the art or as described herein, or those generated by a variety of methods known in the art (see, e.g., U.S. Pat. Nos. 6,291,161 and 6,291,158). For example, fusion protein binding domains may be identified by screening a Fab phage library for Fab fragments that specifically bind to a target of interest (see Hoet et al., Nat. Biotechnol. 23:344, 2005). Additionally, traditional strategies for hybridoma development, such as using a target antigen as an immunogen in convenient systems (e.g., mice, HuMAb Mouse®, TC Mouse™, KM-Mouse®, llamas, sheep, chicken, rats, hamsters, rabbits, etc.), can be used to develop anti-target antibodies having target-specific binding domains of interest.

Sources of further binding domains include target-specific antibody variable domains from various species (which can be formatted as antibodies, sFvs, scFvs, Fabs, or soluble VH domain or domain antibodies), including human, rodent, avian, and ovine. Additional sources of binding domains include variable domains of antibodies from other species, such as camelid (from camels, dromedaries, or llamas (Ghahroudi et al., FEBS Letters 414:521, 1997; Vincke et al., J. Biol. Chem. 284:3273, 2009; and Hamers-Casterman et al., Nature 363:446, 1993; and Nguyen et al., J. Mol. Biol. 275:413, 1998), nurse sharks (Roux et al., Proc. Nat'l. Acad. Sci. (USA) 95:11804, 1998), spotted ratfish (Nguyen et al., Immunogenetics 54:39, 2002), or lamprey (Herrin et al., Proc. Nat'l. Acad. Sci. (USA) 105:2040, 2008 and Alder et al., Nature Immunol. 9:319, 2008). These antibodies can apparently form antigen-binding regions using only heavy chain variable region, i.e., these functional antibodies are homodimers of heavy chains only (referred to as “heavy chain antibodies”) (Jespers et al., Nat. Biotechnol. 22:1161, 2004; Cortez-Retamozo et al., Cancer Res. 64:2853, 2004; Baral et al., Nature Med. 12:580, 2006, and Barthelemy et al., J. Biol. Chem. 283:3639, 2008).

Other alternative sources of target-specific binding domains includes sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), ankyrin repeat proteins (also known as DARPins; Binz et al., J. Mol. Biol. 332:489, 2003 and Binz et al., Nat. Biotechnol. 22:575, 2004), fibronectin binding domains (also known as adnectins or monobodies; Richards et al., J. Mol. Biol. 326:1475, 2003; Parker et al., Protein Eng. Des. Sel. 18:435, 2005 and Hackel et al., J. Mol. Biol. 381:1238, 2008), cysteine-knot miniproteins (Vita et al., Proc. Nat'l. Acad. Sci. (USA) 92:6404, 1995; Martin et al., Nat. Biotechnol. 21:71, 2002 and Huang et al., Structure 13:755, 2005), tetratricopeptide repeat domains (Main et al., Structure 11:497, 2003 and Cortajarena et al., ACS Chem. Biol. 3:161, 2008), leucine-rich repeat domains (Stumpp et al., J. Mol. Biol. 332:471, 2003), anticalins (Skerra, FEBS J. 275:2677, 2008), lipocalin domains (see, e.g., PCT Publication No. WO 2006/095164, Beste et al., Proc. Nat'l. Acad. Sci. (USA) 96:1898, 1999 and Schönfeld et al., Proc. Nat'l. Acad. Sci. (USA) 106:8198, 2009), armadillo repeat proteins (ArmRPs; Varadamsetty et al., J. Mol. Biol. 424:68, 2012), diabodies (Manzke et al., Int. J. Cancer 82:700, 1999), repebodies (Lee et al., Proc. Nat'l. Acad. Sci. U.S.A. 109: 3299, 2012), minibodies (Hu et al., Cancer Res. 56:3055, 1996), cyclotides (Craik et al., J. Mol. Biol. 294:1327, 1999), V-like domains (see, e.g., US Patent Application Publication No. 2007/0065431), C-type lectin domains (Zelensky and Gready, FEBS J. 272:6179, 2005; Beavil et al., Proc. Nat'l. Acad. Sci. (USA) 89:753, 1992 and Sato et al., Proc. Nat'l. Acad. Sci. (USA) 100:7779, 2003), mAb² or Fcab™ (see, e.g., PCT Publication Nos. WO 2007/098934; WO 2006/072620), or the like (Nord et al., Protein Eng. 8:601, 1995; Nord et al., Nat. Biotechnol. 15:772, 1997; Nord et al., Eur. J. Biochem. 268:4269, 2001; and Binz et al. (2005) Nat. Biotechnol. 23:1257, 2005).

In further embodiments, a binding domain is specific for a target that is an antigen associated with a cancer (e.g., solid malignancy, hematologic malignancy), an inflammatory disease, an autoimmune disease, or a graft versus host disease. Exemplary target antigens include, alpha folate receptor (FRα), αᵥβ₆ integrin, ADGRE2, BACE2, B cell maturation antigen (BCMA), B7-H3 (CD276), B7-H4, B7-H6, CA19.9, carbonic anhydrase IX (CAIX), CCR1, CD7, CD16, CD19, CD20, CD22, CD30, CD33, CD37, CD38, CD44, CD44v6, CD44v7/8, CD70, CD79a, CD79b, CD123, CD133, CD138, CD171, CD244, carcinoembryonic antigen (CEA), C-type lectin-like molecule-1 (CLL1), CD2 subset 1 (CS-1), CLDN6, cMET, chondroitin sulfate proteoglycan 4 (CSPG4), CLDN18.2, cutaneous T cell lymphoma-associated antigen 1 (CTAGE1), DLL3, epidermal growth factor receptor (EGFR), epidermal growth factor receptor variant III (EGFRvIII), EGFR806, epithelial glycoprotein 2 (EGP2), epithelial glycoprotein 40 (EGP40), EPHB2, ERBB4, epithelial cell adhesion molecule (EPCAM), ephrin type-A receptor 2 (EPHA2), fibroblast activation protein (FAP), Fc Receptor Like 5 (FCRLS), fetal acetylcholinesterase receptor (AchR), FLT3, FN, FN-EDB, FRBeta, ganglioside G2 (GD2), ganglioside G3 (GD3), Glypican-3 (GPC3), EGFR family including ErbB2 (HER2), HER2p95, EGFRv3, IL-10□1, IL-13□12, Kappa, cancer/testis antigen 2 (LAGE-1A), K-Ras, K-Ras G12C, K-Ras G12D, K-Ras G12V, Lambda, Lewis-Y (LeY), L1 cell adhesion molecule (L1-CAM), LILRB2, LY6G6GD, melanoma antigen recognized by T cells 1 (MelanA or MART1), Mesothelin (MSLN), MMP10, MUC1, MUC16, MHC class I chain related proteins A (MICA), MHC class I chain related proteins B (MICB), neural cell adhesion molecule (NCAM), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), transmembrane activator and CAML interactor (TACI), tumor endothelial marker 1 (TEM1/CD248), tumor endothelial marker 7-related (TEM7R), TIM3, trophoblast glycoprotein (TPBG), UL16-binding protein (ULBP) 1, ULBP2, ULBP3, ULBP4, ULBP5, ULBP6, and vascular endothelial growth factor receptor 2 (VEGFR2).

In some embodiments, the one or more antigen-binding domains bind CD19, CD20, CD22, CD33, CD79A, CD79B, B7H3, Muc16, Her2, EGFR, FN-EDB, CLDN18.2, DLL3, FLT3, CLL1, CD123, or BCMA. In some embodiments, the one or more antigen-binding domains bind CD33, CLL1, CD19, CD20, CD22, CD79A, CD79B, or BCMA. In some embodiments, the one or more antigen-binding domains bind CD33 and/or CLL1. In particular embodiments, the binding domain is an anti-CD33 VHH antibody. In particular embodiments, the binding domain is an anti-CLL1 VHH antibody.

In various embodiments, the one or more antigen-binding domains bind a target polypeptide derived from a protein selected from the group including: α-fetoprotein (AFP), ASCL2, B Melanoma Antigen (BAGE) family members, Brother of the regulator of imprinted sites (BORIS), Cancer-testis antigens, Cancer-testis antigen 83 (CT-83), Carbonic anhydrase IX (CAIX), Carcinoembryonic antigen (CEA), Cytomegalovirus (CMV) antigens, Cytotoxic T cell (CTL)-recognized antigen on melanoma (CAMEL), Epstein-Barr virus (EBV) antigens, EPHB2, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human papillomavirus (HPV)-E6, HPV-E7, Human telomerase reverse transcriptase (hTERT), IGF2BP3/A3, IGF2BP1, K-Ras, K-Ras G12C, K-Ras G12D, K-Ras G12V, Latent membrane protein 2 (LMP2), LY6G6D, Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, PAP, PIK3CA, PIK3CA H1047R, Placenta-specific 1 (PLAC1), Preferentially expressed antigen in melanoma (PRAME), Prostate specific antigen PSA, Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, TP53 R175H, Tyrosinase, Tyrosinase related protein (TRP)1, TRP2, UBD, Wilms tumor protein (WT-1), Wnt10A, X Antigen Family Member 1 (XAGE1), and X Antigen Family Member 2 (XAGE2).

(iii-c) Intracellular Components.

An intracellular component of a fusion protein includes one or more intracellular signaling, co-stimulatory, or co-receptor domains that transmit or allow the transmission of an intracellular signal. In particular embodiments, the intracellular component generates a signal that promotes an immune effector function of a fusion protein modified cell. In particular embodiments, the intracellular component generates a stimulatory and/or co-stimulatory signal based on ligand binding. Examples of immune effector function include cytolytic activity and helper activity, including the secretion of cytokines. Intracellular component signals can also lead to immune cell proliferation, activation, differentiation, and the like.

A signaling domain refers to the functional portion of a protein which acts by transmitting information within the cell to regulate cellular activity via defined signaling pathways by generating second messengers or functioning as effectors by responding to such messengers. Stimulation refers to a primary response induced by binding of a stimulatory molecule (e.g., a fusion protein) or co-stimulatory molecule with its cognate ligand, thereby mediating a signal transduction event, such as signal transduction via appropriate signaling domains of the fusion protein. Stimulation can mediate altered expression of certain molecules.

An intracellular signaling domain can include the entire intracellular portion of a signaling domain or a functional fragment thereof. In particular embodiments, an intracellular signaling domain can include a primary intracellular signaling domain. In particular embodiments, primary intracellular signaling domains include those derived from the molecules responsible for primary stimulation, or antigen dependent stimulation. In particular embodiments, the intracellular signaling domain can include a costimulatory intracellular domain.

A primary intracellular signaling domain can include a signaling motif which is known as an immunoreceptor tyrosine-based activation motif or ITAM. Examples of ITAM containing primary cytoplasmic signaling sequences include those derived from CD3ζ, common FcR gamma (FCER1G), Fc gamma RIIa, FcR beta (Fc Epsilon R1b), CD3 gamma, CD3 delta, CD3 epsilon, CD79a, CD79b, DAP10, and DAP12.

In particular embodiments, a CD3ζ (CD247) stimulatory domain can include amino acid residues from the cytoplasmic domain of the T cell receptor zeta chain, or functional fragments thereof, that are sufficient to functionally transmit an initial signal necessary for cell activation. In particular embodiments, a CD3ζ stimulatory domain can include a human CD3ζ stimulatory domain or functional fragments thereof. In particular embodiments, in the case of an intracellular signaling domain that is derived from a CD3ζ molecule, the intracellular signaling domain retains sufficient CD3ζ structure such that it can generate a signal under appropriate conditions.

In particular embodiments, the intracellular signaling domain can include a costimulatory intracellular domain. In particular embodiments, costimulatory intracellular signaling domains include those derived from molecules responsible for costimulatory signals, or antigen independent stimulation. In particular embodiments, a costimulatory intracellular signaling domain can be the intracellular portion of a costimulatory molecule. A costimulatory molecule refers to a cognate binding partner on an immune cell that specifically binds with a costimulatory ligand, thereby mediating a costimulatory response by the immune cell, such as proliferation. Costimulatory molecules include cell surface molecules other than antigen receptors or their ligands that contribute to an efficient immune response. A costimulatory molecule can be represented in the following protein families: TNF receptor proteins, immunoglobulin-like proteins, cytokine receptors, integrins, signaling lymphocytic activation molecules (SLAM proteins), and activating NK cell receptors. Examples of such molecules include: an MHC class I molecule, B and T cell lymphocyte attenuator (BTLA, CD272), a Toll ligand receptor, CD27, CD28, 4-1BB (CD137), OX40, GITR, CD30, CD40, ICOS (CD278), BAFFR, HVEM (LIGHTR), ICAM-1, lymphocyte function-associated antigen-1 (LFA-1; CD11a/CD18), CD2, CDS, CD7, CD287, LIGHT, NKG2C, NKG2D, SLAMF7, NKp80 (KLRF1), NKp30, NKp44, NKp46, CD160 (BY55), B7-H3 (CD276), CD19, CD4, CD8α, CD8β, IL2Rβ, IL2Rγ, IL7Rα, ITGA4, VLA1, CD49a, IA4, CD49d, ITGA6, VLA-6, CD49f, ITGAD, CD11d, ITGAE, CD103, ITGAL, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, and the like.

In particular embodiments, a costimulatory intracellular signaling domain (also referred to as costimulatory domain) includes 4-1BB (CD137, TNFRSF9). 4-1BB refers to a member of the tumor necrosis factor receptor (TNFR) superfamily. In particular embodiments, a 4-1BB costimulatory domain includes a human 4-1BB costimulatory domain or a functional fragment thereof.

In particular embodiments, a costimulatory intracellular signaling domain includes CD28. CD28 is a T cell-specific glycoprotein involved in T cell activation, the induction of cell proliferation and cytokine production, and promotion of T cell survival. In particular embodiments, a CD28 costimulatory domain includes a human CD28 costimulatory domain or a functional fragment thereof.

In particular embodiments, an intracellular component includes a combination of one or more stimulatory domains and one or more costimulatory domains described herein. In particular embodiments, an intracellular component includes a 4-1BB costimulatory domain and a CD3ζ stimulatory domain. In particular embodiments, an intracellular component includes a 4-1BB costimulatory domain and a CD3ζ stimulatory domain.

In particular embodiments, an intracellular component includes a CD3ζ primary intracellular signaling domain and an OX40 costimulatory intracellular domain. In particular embodiments, an intracellular component includes a CD3ζ primary intracellular signaling domain and an TNFR2 costimulatory intracellular domain.

(iii-d) Transmembrane Domains.

A fusion protein can be designed to include a transmembrane domain. A transmembrane domain can anchor a fusion protein to a cell membrane. A transmembrane domain can include one or more additional amino acids adjacent to the transmembrane region, e.g., one or more amino acids associated with the extracellular region of the protein from which the transmembrane was derived (e.g., 1, 2, 3, 4, 5, 6, 7, 8, 9, 10, 11, 12, 13, 14, 15 amino acids, or more of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15 amino acids, or more of the intracellular region). In particular embodiments, the transmembrane domain may be from the same protein that an intracellular component signaling domain, costimulatory domain, hinge domain, or co-receptor is derived from. In particular embodiments, the transmembrane domain is not derived from the same protein that any other domain of a fusion protein is derived from. In particular embodiments, the transmembrane domain can be selected or modified by amino acid substitution to avoid binding of or to minimize interactions with other domains in the fusion protein.

In particular embodiments, a transmembrane domain has a three-dimensional structure that is thermodynamically stable in a cell membrane, and generally ranges in length from 15 to 30 amino acids. The structure of a transmembrane domain can include an alpha helix, a beta barrel, a beta sheet, a beta helix, or any combination thereof.

The transmembrane domain may be derived either from a natural or from a recombinant source. Where the source is natural, the domain may be derived from any membrane-bound or transmembrane protein. In particular embodiments, the transmembrane domain is capable of signaling to the intracellular component(s) whenever a fusion protein having an extracellular ligand binding domain has bound to a target. In particular embodiments, a transmembrane domain may include at least the transmembrane region(s) of: the α, β, or ζ chain of the T-cell receptor; CD28; CD27; CD3ε; CD45; CD4; CD5; CD8; CD9; CD16; CD22; CD33; CD37; CD64; CD80; CD86; CD134; CD137; and/or CD154. In particular embodiments, a transmembrane domain may include at least the transmembrane region(s) of: KIRDS2; OX40; CD2; LFA-1; ICOS; 4-1BB; GITR; CD40; BAFFR; HVEM; SLAMF7; NKp80; NKp44; NKp30; NKp46; CD160; CD19; IL2Rβ; IL2Rγ; IL7Ra; ITGA1; VLA1; CD49a; ITGA4; IA4; CD49D; ITGA6; VLA-6; CD49f; ITGAD; CDI Id; ITGAE; CD103; ITGAL; CDI Ia; ITGAM; CDI Ib; ITGAX; CDI Ic; ITGB1; CD29; ITGB2; CD18; ITGB7; TNFR2; DNAM1; SLAMF4; CD84; CD96; CEACAM1; CRT AM; Ly9; CD160; PSGL1; CD100; SLAMF6 (NTB-A, LyI08); SLAM; BLAME; SELPLG; LTBR; PAG/Cbp; NKG2D; and/or NKG2C. In particular embodiments, a transmembrane domain may include a transmembrane domain from CD28, CD4, or the CD8α chain.

In particular embodiments, the transmembrane domain can include predominantly hydrophobic residues such as leucine and valine. In particular embodiments, the transmembrane domain can include a triplet of phenylalanine, tryptophan and valine found at each end of the transmembrane domain. In particular embodiments, a CD28, CD4, or CD8 hinge is juxtaposed on the extracellular side of the transmembrane domain.

In some embodiments, a fusion protein (e.g., DARIC binding component) includes a transmembrane domain or GPI signal sequence. In further embodiments, a fusion protein (e.g., DARIC binding component) contains a GPI molecule, wherein the GPI signal sequence has been removed or altered to attach the GPI molecule.

In particular embodiments, the first and/or second fusion protein includes a transmembrane domain including a CD4 transmembrane domain. In particular embodiments, the first and/or second fusion protein includes a transmembrane domain including a CD8α transmembrane domain.

(iii-e) Linkers.

As used herein, a linker within a fusion protein can be any portion of a fusion protein that serves to connect two subcomponents or domains of the fusion protein. In particular embodiments, linkers can provide flexibility for different components of the fusion protein. Linkers in the context of linking VH and VL of antibody derived binding domains of scFv are described above. Linkers can also include spacer regions and junction amino acids.

Spacer regions are a type of linker region that are used to create appropriate distances and/or flexibility from other linked components.

In particular embodiments, the length of a spacer region can be customized for individual purposes. For example, a spacer region can be customized for individual cellular markers on targeted cells to optimize cell recognition and destruction following fusion protein binding. In certain examples, the spacer can be of a length that provides for increased responsiveness of a fusion protein expressing cell following antigen binding, as compared to in the absence of the spacer. In particular embodiments, a spacer region length can be selected based upon the location of a cellular marker epitope, affinity of a binding domain for the epitope, and/or the ability of the fusion protein modified cells to destroy target cells ex vivo and/or in vivo in response to cellular marker recognition. Spacer regions can also allow for high expression levels in fusion protein modified cells. In particular embodiments, an extracellular spacer region of a fusion protein is located between a transmembrane domain and the extracellular binding domain.

Exemplary spacers include those having 10 to 250 amino acids, 10 to 200 amino acids, 10 to 150 amino acids, 10 to 100 amino acids, 10 to 50 amino acids, or 10 to 25 amino acids. In particular embodiments, a spacer region is 12 amino acids, 20 amino acids, 21 amino acids, 26 amino acids, 27 amino acids, 45 amino acids, or 50 amino acids. In particular embodiments, a long spacer is greater than 119 amino acids, an intermediate spacer is 13-119 amino acids, and a short spacer is 10-12 amino acids.

In particular embodiments, a spacer region includes an immunoglobulin hinge region. An immunoglobulin hinge region may be a wild-type immunoglobulin hinge region or an altered wild-type immunoglobulin hinge region. In particular embodiments, an immunoglobulin hinge region is a human immunoglobulin hinge region. An immunoglobulin hinge region may be an IgG, IgA, IgD, IgE, or IgM hinge region. An IgG hinge region may be an IgG1, IgG2, IgG3, or IgG4 hinge region. In particular embodiments, the spacer region can include all or a portion of a hinge region sequence from IgG1, IgG2, IgG3, IgG4 or IgD alone or in combination with all or a portion of a CH2 region; all or a portion of a CH3 region; or all or a portion of a CH2 region and all or a portion of a CH3 region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

Exemplary spacers include IgG4 hinge alone, IgG4 hinge linked to CH2 and CH3 domains, or IgG4 hinge linked to the CH3 domain. Hinge regions can be modified to avoid undesirable structural interactions such as dimerization with unintended partners. Other examples of hinge regions that can be used in fusion proteins described herein include the hinge region present in extracellular regions of type 1 membrane proteins, such as CD8α, CD4, CD28, and CD7, which may be wild-type or variants thereof.

In particular embodiments, a spacer region includes a hinge region of a type II C-lectin interdomain (stalk) region or a cluster of differentiation (CD) molecule stalk region. A “stalk region” of a type II C-lectin or CD molecule refers to the portion of the extracellular domain of the type II C-lectin or CD molecule that is located between the C-type lectin-like domain (CTLD; e.g., similar to CTLD of natural killer cell receptors) and the hydrophobic portion (transmembrane domain). For example, the extracellular domain of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids between the stalk region and the transmembrane region or the CTLD. In another example, the 233 amino acid human NKG2A protein (UniProt ID P26715.1) has a hydrophobic portion (transmembrane domain) ranging from amino acids 71-93 and an extracellular domain ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

(iii-f) Tags and Selectable Markers.

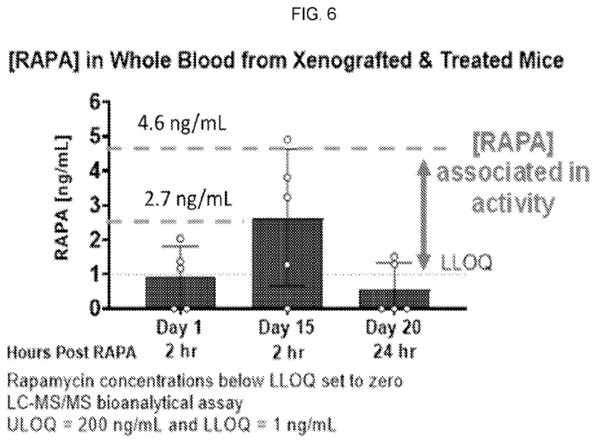

In particular embodiments, a fusion protein can include one or more tags and/or be expressed with one more selectable markers. Exemplary tags include His tag, Flag tags, Xpress tag, Avi tag, Calmodulin binding peptide (CBP) tag, Polyglutamate tag, HA tags, Myc tag, Strep tag (which refers to the original STREP® tag, STREP® tag II (IBA Institut fur Bioanalytik, Germany); see, e.g., U.S. Pat. No. 7,981,632), Softag 1, Softag 3, and V5. See FIG. 6 for exemplary sequences.

Conjugate binding molecules that specifically bind tag sequences disclosed herein are commercially available. For example, His tag antibodies are commercially available from suppliers including Life Technologies, Pierce Antibodies, and GenScript. Flag tag antibodies are commercially available from suppliers including Pierce Antibodies, GenScript, and Sigma-Aldrich. Xpress tag antibodies are commercially available from suppliers including Pierce Antibodies, Life Technologies, and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal, and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

In particular embodiments, one or more transduction markers can be co-expressed with the fusion protein, for example, using a skipping element or IRES site that allows expression of the transduction marker and other components of the fusion protein as distinct molecules. Exemplary self-cleaving polypeptides include 2A peptides from porcine teschovirus-1 (P2A), Thosea asigna virus (T2A), equine rhinitis A virus (E2A), and foot-and-mouth disease virus (F2A) as described elsewhere herein.

In particular embodiments, the transduction marker can include any cell surface displayed marker that can be detected with an antibody that binds to that marker and allows sorting of cells that have the marker. In particular embodiments, the transduction marker can include the magnetic sortable marker streptavidin binding peptide (SBP) displayed at the cell surface by a truncated Low Affinity Nerve Growth Receptor (LNGFRF) and one-step selection with streptavidin-conjugated magnetic beads (Matheson et al. (2014) PloS one 9(10): e111437) or a truncated human epidermal growth factor receptor (EGFR) (tEGFR; see Wang et al., Blood 118: 1255, 2011).

In some alternatives, the transduction marker is a truncated EGFR (EGFRt), a truncated Her2 (Her2), a truncated Her2 (Her2tG), a truncated CD19 (CD19t), or the transduction marker DHFRdm.

Transduction markers can include any suitable fluorescent protein including: blue fluorescent proteins (e.g., BFP, eBFP, eBFP2); cyan fluorescent proteins (e.g., eCFP, Cerulean, CyPet); green fluorescent proteins (e.g., GFP-2, tagGFP, turboGFP, eGFP,); orange fluorescent proteins (e.g., mOrange, mKO, Kusabira-Orange); red fluorescent proteins (e.g., mKate, mPlum, DsRed monomer, mCherry, mRFP1, DsRed-Express); yellow fluorescent proteins (e.g., YFP, eYFP, Citrine, Venus); and any other suitable fluorescent proteins, including, for example, firefly Iuciferase.

### (iv) POLYNUCLEOTIDES

In particular embodiments, polynucleotides encoding a DARIC, one or more DARIC components, DARIC signaling components, and/or DARIC binding components.

As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include: pre-messenger RNA (pre-mRNA), messenger RNA (mRNA), RNA, short interfering RNA (siRNA), short hairpin RNA (shRNA), microRNA (miRNA), ribozymes, genomic RNA (gRNA), plus strand RNA (RNA(+)), minus strand RNA (RNA(−)), tracrRNA, crRNA, single guide RNA (sgRNA), synthetic RNA, synthetic mRNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

As used herein, “isolated polynucleotide” refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” also refers to a complementary DNA (cDNA), a recombinant DNA, or other polynucleotide that does not exist in nature and that has been made by the hand of man. In particular embodiments, an isolated polynucleotide is a synthetic polynucleotide, a semi-synthetic polynucleotide, or a polynucleotide obtained or derived from a recombinant source.

In various embodiments, a polynucleotide includes an mRNA encoding a polypeptide contemplated herein. In certain embodiments, the mRNA includes a cap, one or more nucleotides, and a poly(A) tail.

In particular embodiments, polynucleotides encoding one or more DARIC components may be codon-optimized. As used herein, the term “codon-optimized” refers to substituting codons in a polynucleotide encoding a polypeptide in order to increase the expression, stability and/or activity of the polypeptide. Factors that influence codon optimization include one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, and/or (xi) isolated removal of spurious translation initiation sites.

As used herein the term “nucleotide” refers to a heterocyclic nitrogenous base in N-glycosidic linkage with a phosphorylated sugar. Nucleotides are understood to include natural bases, and a wide variety of art-recognized modified bases. Such bases are generally located at the 1′ position of a nucleotide sugar moiety. Nucleotides generally include a base, sugar and a phosphate group. In ribonucleic acid (RNA), the sugar is a ribose, and in deoxyribonucleic acid (DNA) the sugar is a deoxyribose, i.e., a sugar lacking a hydroxyl group that is present in ribose.

In various illustrative embodiments, polynucleotides contemplated herein include polynucleotides encoding one or more DARIC components, engineered antigen receptors, fusion polypeptides, and expression vectors, viral vectors, and transfer plasmids including polynucleotides contemplated herein.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may include 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. The cassette can be removed and inserted into a plasmid or viral vector as a single unit.

Polynucleotides include polynucleotide(s)-of-interest. As used herein, the term “polynucleotide-of-interest” refers to a polynucleotide encoding a polypeptide or fusion polypeptide or a polynucleotide that serves as a template for the transcription of an inhibitory polynucleotide, as contemplated herein.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated, expressed and/or delivered using any of a variety of well-established techniques known and available in the art. In order to express a desired polypeptide, a nucleotide sequence encoding the polypeptide, can be inserted into appropriate vector.

Illustrative examples of vectors include plasmids, autonomously replicating sequences, and transposable elements, e.g., Sleeping Beauty, PiggyBac.

Additional Illustrative examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include pCIneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

“Expression control sequences,” “control elements,” or “regulatory sequences” present in an expression vector are those non-translated regions of the vector including an origin of replication, selection cassettes, promoters, enhancers, translation initiation signals (Shine Dalgarno sequence or Kozak sequence) introns, a polyadenylation sequence, 5′ and 3′ untranslated regions, all of which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a polynucleotide includes a vector, including expression vectors and viral vectors. A vector may include one or more exogenous, endogenous, or heterologous control sequences such as promoters and/or enhancers. An “endogenous control sequence” is one which is naturally linked with a given gene in the genome. An “exogenous control sequence” is one which is placed in juxtaposition to a gene by means of genetic manipulation (i.e., molecular biological techniques) such that transcription of that gene is directed by the linked enhancer/promoter. A “heterologous control sequence” is an exogenous sequence that is from a different species than the cell being genetically manipulated. A “synthetic” control sequence may include elements of one more endogenous and/or exogenous sequences, and/or sequences determined in vitro or in silico that provide optimal promoter and/or enhancer activity for the particular therapy.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells include an AT-rich region located 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, dl587rev primer-binding site substituted (MND) U3 promoter (Haas et al. Journal of Virology. 2003; 77(17): 9439-9450).

In one embodiment, a vector includes an MNDU3 promoter.

In one embodiment, a vector includes an EF1a promoter including the first intron of the human EF1a gene.

In one embodiment, a vector includes an EF1a promoter that lacks the first intron of the human EF1a gene.

In a particular embodiment, it may be desirable to use a cell, cell type, cell lineage or tissue specific expression control sequence to achieve cell type specific, lineage specific, or tissue specific expression of a desired polynucleotide sequence (e.g., to express a particular nucleic acid encoding a polypeptide in only a subset of cell types, cell lineages, or tissues or during specific stages of development).

In a particular embodiment, it may be desirable to express a polynucleotide a T cell specific promoter.

As used herein, “conditional expression” may refer to any type of conditional expression including inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc. Inducer agents include glucocorticoids, estrogens, mifepristone (RU486), metals, interferons, small molecules, cumate, tetracycline, doxycycline, and variants thereof.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. Examples of IRES generally employed by those of skill in the art include those described in U.S. Pat. No. 6,692,736. Further examples of “IRES” known in the art include IRES obtainable from picornavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18(11):6178-6190), the fibroblast growth factor 2 (FGF-2), and insulin-like growth factor (IGFII), the translational initiation factor eIF4G and yeast transcription factors TFIID and HAP4, the encephelomycarditis virus (EMCV) which is commercially available from Novagen (Duke et al., 1992. J. Virol 66(3):1602-9) and the VEGF IRES (Huez et al., 1998. Mol Cell Biol 18(11):6178-90). IRES have also been reported in viral genomes of Picornaviridae, Dicistroviridae and Flaviviridae species and in HCV, Friend murine leukemia virus (FrMLV) and Moloney murine leukemia virus (MoMLV).

In one embodiment, the IRES used in polynucleotides contemplated herein is an EMCV IRES.

In particular embodiments, the polynucleotides a consensus Kozak sequence. As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO: 59), where R is a purine (A or G) (Kozak, 1986. Cell. 44(2):283-92, and Kozak, 1987. Nucleic Acids Res. 15(20):8125-48).

Elements directing the efficient termination and polyadenylation of the heterologous nucleic acid transcripts increases heterologous gene expression. Transcription termination signals are generally found downstream of the polyadenylation signal. In particular embodiments, vectors include a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments, the poly(A) sequence is an SV40 polyA sequence, a bovine growth hormone polyA sequence (BGHpA), a rabbit β-globin polyA sequence (rβgpA), variants thereof, or another suitable heterologous or endogenous polyA sequence known in the art. In particular embodiments, the poly(A) sequence is synthetic.

In particular embodiments, polynucleotides encoding one or more polypeptides, or fusion polypeptides may be introduced into immune effector cells, e.g., T cells, by both non-viral and viral methods. In particular embodiments, delivery of one or more polynucleotides may be provided by the same method or by different methods, and/or by the same vector or by different vectors.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to a T cell.

Illustrative examples of non-viral vectors include plasmids (e.g., DNA plasmids or RNA plasmids), transposons, cosmids, and bacterial artificial chromosomes.

Illustrative methods of non-viral delivery of polynucleotides contemplated in particular embodiments include: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term “viral vector” or “lentiviral vector” may refer either to a virus or viral particle capable of transferring a nucleic acid into a cell or to the transferred nucleic acid itself. Viral vectors and transfer plasmids contain structural and/or functional genetic elements that are primarily derived from a virus.

Viral vectors including polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, viral vectors including polynucleotides contemplated herein are administered directly to an organism or subject for transduction of cells in vivo. Alternatively, naked DNA can be administered. Administration is by any of the routes normally used for introducing a molecule into ultimate contact with blood or tissue cells including injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated in particular embodiments include adeno-associated virus (AAV), retrovirus, herpes simplex virus, adenovirus, and vaccinia virus vectors.

In various embodiments, one or more polynucleotides encoding one or more DARIC components and/or other polypeptides contemplated herein are introduced into an immune effector cell, e.g., T cell, by transducing the cell with a recombinant adeno-associated virus (rAAV), including the one or more polynucleotides.

AAV is a small (26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5 and 3′ AAV inverted terminal repeats (ITRs). The ITR sequences are 145 bp in length. In particular embodiments, the rAAV includes ITRs and capsid sequences isolated from AAV1, AAV2, AAV3, AAV4, AAV5, AAV6, AAV7, AAV8, AAV9, or AAV10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may include ITRs from AAV2, and capsid proteins from any one of AAV1, AAV2, AAV3, AAV4, AAV5, AAV6, AAV7, AAV8, AAV9, or AAV10. In a preferred embodiment, the rAAV includes ITR sequences derived from AAV2 and capsid sequences derived from AAV6. In a preferred embodiment, the rAAV includes ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

In various embodiments, one or more polynucleotides encoding one or more DARIC components and/or other polypeptides contemplated herein are introduced into an immune effector cell, e.g., T cell, by transducing the cell with a retrovirus, e.g., lentivirus, including the one or more polynucleotides.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

In various embodiments, a lentiviral vector contemplated herein includes one or more LTRs, and one or more, or all, of the following accessory elements: a cPPT/FLAP, a Psi (Ψ) packaging signal, an export element, poly (A) sequences, and may optionally include a WPRE or HPRE, an insulator element, a selectable marker, and a cell suicide gene, as discussed elsewhere herein.

In particular embodiments, lentiviral vectors contemplated herein may be integrative or non-integrating or integration defective lentivirus. As used herein, the term “integration defective lentivirus” or “IDLV” refers to a lentivirus having an integrase that lacks the capacity to integrate the viral genome into the genome of the host cells. Integration-incompetent viral vectors have been described in patent application WO 2006/010834, which is herein incorporated by reference in its entirety.

Illustrative mutations in the HIV-1 pol gene suitable to reduce integrase activity include: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H.

The term “long terminal repeat (LTR)” refers to domains of base pairs located at the ends of retroviral DNAs which, in their natural sequence context, are direct repeats and contain U3, R and U5 regions.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173.

As used herein, the term “packaging signal” or “packaging sequence” refers to psi [Ψ] sequences located within the retroviral genome which are required for insertion of the viral RNA into the viral capsid or particle, see e.g., Clever et al., 1995. J. of Virology, Vol. 69, No. 4; pp. 2101-2109.

The term “export element” refers to a cis-acting post-transcriptional regulatory element which regulates the transport of an RNA transcript from the nucleus to the cytoplasm of a cell. Examples of RNA export elements include the human immunodeficiency virus (HIV) rev response element (RRE) (see e.g., Cullen et al., 1991. J. Virol. 65:1053; and Cullen et al., 1991. Cell 58: 423), and the hepatitis B virus post-transcriptional regulatory element (HPRE).

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766).

Lentiviral vectors preferably contain several safety enhancements as a result of modifying the LTRs. “Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. Self-inactivation is preferably achieved through in the introduction of a deletion in the U3 region of the 3′ LTR of the vector DNA, i.e., the DNA used to produce the vector RNA. Thus, during reverse transcription, this deletion is transferred to the 5′ LTR of the proviral DNA. In particular embodiments, it is desirable to eliminate enough of the U3 sequence to greatly diminish or abolish altogether the transcriptional activity of the LTR, thereby greatly diminishing or abolishing the production of full-length vector RNA in transduced cells. In the case of HIV based lentivectors, it has been discovered that such vectors tolerate significant U3 deletions, including the removal of the LTR TATA box (e.g., deletions from −418 to −18), without significant reductions in vector titers.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells.

In certain embodiments, lentiviral vectors are produced according to known methods. See e.g., Kutner et al., BMC Biotechnol. 2009; 9:10. doi: 10.1186/1472-6750-9-10; Kutner et al. Nat. Protoc. 2009; 4(4):495-505. doi: 10.1038/nprot.2009.22.

According to certain specific embodiments contemplated herein, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides encoding one or more DARIC components and/or other polypeptides contemplated herein are introduced into an immune effector cell, by transducing the cell with an adenovirus including the one or more polynucleotides.

Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et aL, 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In various embodiments, one or more polynucleotides encoding one or more DARIC components and/or other polypeptides contemplated herein are introduced into an immune effector cell by transducing the cell with a herpes simplex virus, e.g., HSV-1, HSV-2, including the one or more polynucleotides.

The mature HSV virion includes an enveloped icosahedral capsid with a viral genome including a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group including: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which are incorporated by reference herein in its entirety.

### (v) GENETICALLY MODIFIED CELLS

The present disclosure includes cells genetically modified to express a DARIC. As used herein, the term “genetically modified”, “edited”, or “genetically engineered” refers to the addition of extra genetic material in the form of DNA or RNA into the cell. The terms “genetically modified cells” and “modified cells” are used interchangeably. In particular embodiments, a cell genetically modified to express a DARIC or components thereof includes an immune effector cell. An “immune effector cell” includes any cell of the immune system that has one or more effector functions (e.g., cytotoxic cell killing activity, secretion of cytokines, induction of antibody-dependent cell cytotoxicity (ADCC) and/or complement-dependent cytotoxicity (CDC). Immune effector cells are a subtype of immune cells.

The term “express” or “expression” refers to the process by which a polypeptide is produced based on the nucleic acid sequence of a gene.

Immune cells of the disclosure can be autologous/autogeneic (“self”) or non-autologous (“non-self,” e.g., allogeneic, syngeneic or xenogeneic). “Autologous” refers to cells from the same subject. “Allogeneic” refers to cells of the same species that differ genetically to a cell in comparison. “Syngeneic” refers to cells of a different subject that are genetically identical to the cell in comparison. “Xenogeneic” refers to cells of a different species to the cell in comparison. In particular embodiments, modified cells of the disclosure are autologous or allogeneic.

In particular embodiments, genetically modified cells include lymphocytes. In particular embodiments, genetically modified cells include T cells, B cells, natural killer (NK) cells, monocytes/macrophages, or HSPC.

Most T cells have a T-cell receptor (TCR) composed of two separate peptide chains (the α- and β-TCR chains). γd T cells represent a small subset of T cells that possess a distinct T cell receptor (TCR) made up of one γ-chain and one d-chain.

CD3 is expressed on all mature T cells. T cells can further be classified into cytotoxic T cells (CD8+ T cells, also referred to as CTLs) and helper T cells (CD4+ T cells).

Cytotoxic T cells destroy virally infected cells and tumor cells and are also implicated in transplant rejection. These cells recognize their targets by binding to antigen associated with MHC class I, which is present on the surface of nearly every cell of the body.

Central memory T cells (TCM) refer to antigen experienced CTL that express CD62L or CCR7 and CD45RO and does not express or has decreased expression of CD45RA as compared to naive cells.

Effector memory T cells (TEM) refer to an antigen experienced T-cell that does not express or has decreased expression of CD62L as compared to central memory cells and does not express or has decreased expression of CD45RA as compared to a naive cell. In particular embodiments, effector memory T cells are negative for expression of CD62L and CCR7, compared to naive cells or central memory cells, and have variable expression of CD28 and CD45RA. Effector T cells are positive for granzyme B and perforin as compared to memory or naive T cells.

Helper T cells assist other immune cells such as activating of cytotoxic T cells and macrophages and facilitating the maturation of B cells, among other functions. Helper T cells become activated when they are presented with peptide antigens by MHC class II molecules that are expressed on the surface of antigen presenting cells (APCs). Once activated, they divide rapidly and secrete cytokines that regulate or assist in the active immune response.

Natural killer T (NKT) cells are a subset of T cells that co-express an αβ T-cell receptor, but also express a variety of molecular markers that are typically associated with natural killer cells, such as NK1.1 (CD161), CD16, and/or CD56.

Natural killer cells (also known as K cells and killer cells) express CD8, CD16 and CD56 but do not express CD3. NK cells also express activating receptors such as NKp46 and inhibitory receptors such as NKG2A that regulate NK cell cytotoxic function against tumor and virally infected cells.

Tumor-infiltrating lymphocytes (TILs) refers to immune cells that have moved from the blood into a tumor and can function to recognize and kill cancer cells. Marrow-infiltrating lymphocytes (MILs) are antigen-experienced immune cells that travel to and remain in the bone marrow. Mucosal-associated invariant T (MAIT) cells are innate-like T cells which are found in the mucosa, blood, and secondary lymphoid organs (SLO), and display effector phenotype. MAIT cells display a semi-invariant T cell receptor (TCR) and are restricted by the major histocompatibility complex related molecule, MR1.

Macrophages (and their precursors, monocytes) reside in every tissue of the body where they engulf apoptotic cells, pathogens and other non-self-components. Monocytes/macrophages express CD11b, F4/80, CD68, CD11c, IL-4Rα, and/or CD163.

Immature dendritic cells (i.e., pre-activation) engulf antigens and other non-self-components in the periphery and subsequently, in activated form, migrate to T cell areas of lymphoid tissues where they provide antigen presentation to T cells. Dendritic cells express CD1a, CD1b, CD1c, CD1d, CD21, CD35, CD39, CD40, CD86, CD101, CD148, CD209, and DEC-205.

Hematopoietic stem cells (HSC) refer to undifferentiated hematopoietic cells that are capable of self-renewal and differentiation into all other hematopoietic cell types. HSC are CD34+.

Hematopoietic progenitor cells (HPC) are derived from HSC and are capable of further differentiation into mature cell types. HPC can self-renew or can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T cells, B cells, and NK cells. HPC are CD24^{lo} Lin⁻ CD117⁺.

HSPC refer to a cell population having HSC and HPC. HSPC cell populations can be positive for CD34, CD43, CD45RO, CD45RA, CD59, CD90, CD109, CD117, CD133, CD166, HLA DR, or a combination thereof.

Induced pluripotent stem cells (iPSCs) refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing or contacting with reprogramming factors.

Cells can be genetically modified ex vivo and in vivo by any method known in the art. In particular embodiments, cells are genetically modified using cell-targeted delivery methods.

In particular embodiments, lymphocytes are isolated from a sample such as blood or a blood-derived sample, an apheresis or a leukapheresis product. Exemplary samples include whole blood, peripheral blood mononuclear cells (PBMCs), bone marrow, thymus, cancer tissue, lymphoid tissue, spleen, or other appropriate sources.

Sources of HSPC include, for example, peripheral blood (see U.S. Pat. Nos. 5,004,681; 7,399,633; and U.S. Pat. No. 7,147,626; Craddock, et al., 1997, Blood 90(12):4779-4788; Jin, et al., 2008, Journal of Translational Medicine 6:39; Pelus, 2008, Curr. Opin. Hematol. 15(4):285-292; Papayannopoulou, et al., 1998, Blood 91(7):2231-2239; Tricot, et al., 2008, Haematologica 93(11):1739-1742; and Weaver et al., 2001, Bone Marrow Transplantation 27(2):S23-S29).

Methods regarding collection, anti-coagulation and processing, etc. of blood samples can be found in, for example, Alsever, et al., 1941, N.Y. St. J. Med. 41:126; De Gowin, et al., 1940, J. Am. Med. Ass. 114:850; Smith, et al., 1959, J. Thorac. Cardiovasc. Surg. 38:573; Rous and Turner, 1916, J. Exp. Med. 23:219; and Hum, 1968, Storage of Blood, Academic Press, New York, pp. 26-160.

In particular embodiments, collected cells are washed, centrifuged, and/or incubated in the presence of one or more reagents, for example, to remove unwanted components, enrich for desired components, lyse or remove cells sensitive to particular reagents. The isolation can include one or more of various cell preparation and separation steps, including separation based on one or more properties, such as size, density, sensitivity or resistance to particular reagents, and/or affinity, e.g., immunoaffinity, to antibodies or other binding partners.

In particular embodiments, one or more of the cell populations enriched, isolated and/or selected from a sample by the provided methods are cells that are positive for (marker+) or express high levels (marker^{hi}) of one or more particular markers, such as surface markers, or that are negative for (marker−) or express relatively low levels (marker^{lo}) of one or more markers.

In particular embodiments, T cells can be isolated from peripheral blood mononuclear cells (PBMCs) by lysing the red blood cells and depleting the monocytes, for example, by centrifugation through a PERCOLL™ gradient. In particular embodiments, a specific subpopulation of T cells, expressing CD3, CD28, CD4, CD8, CD45RA, and CD45RO is further isolated by positive or negative selection techniques. In particular embodiments, cell sorting and/or selection occurs via negative magnetic immunoadherence or flow cytometry using a cocktail of monoclonal antibodies directed to cell surface markers present on the cells negatively selected. For example, to enrich for CD4⁺ cells by negative selection, a monoclonal antibody cocktail that typically includes antibodies to CD14, CD20, CD11b, CD16, HLA-DR, and CD8 can be used.

Following isolation and/or enrichment, cells can be expanded to increase the number of cells. In particular embodiments, T cells can be activated and expanded before or after genetic modification to express an activity-inducible fusion protein, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and US 2006/0121005.

Generally, the T cells are expanded by contact with a surface having attached thereto an agent that stimulates a CD3 TCR complex associated signal and a ligand that stimulates a co-stimulatory molecule on the surface of the T cells. In particular embodiments, PBMCs or isolated T cells are contacted with a stimulatory agent and costimulatory agent, such as anti-CD3 and anti-CD28 antibodies, generally attached to a bead or other surface, in a culture medium with appropriate cytokines (see Berg et al., Transplant Proc. 30(8):3975-3977, 1998; Haanen et al., J. Exp. Med. 190(9): 13191328, 1999; Garland et al., J. Immunol Meth. 227(1-2):53-63, 1999). In particular embodiments, the T cells may be activated and stimulated to proliferate with feeder cells and appropriate antibodies and cytokines using methods such as those described in U.S. Pat. Nos. 6,040,177; 5,827,642; and WO 2012/129514.

In particular embodiments, artificial APC (aAPC) can be made by engineering K562, U937, 721.221, T2, and C1R cells to direct the stable expression and secretion of a variety of co-stimulatory molecules and cytokines. aAPCs are described in WO 03/057171 and US 2003/0147869.

In particular embodiments, HSPCs can be isolated and/or expanded following methods described in, for example, U.S. Pat. Nos. 7,399,633; 5,004,681; US 2010/0183564; WO2006/047569; WO2007/095594; WO 2011/127470; and WO 2011/127472; Vamum-Finney, et al., 1993, Blood 101:1784-1789; Delaney, et al., 2005, Blood 106:2693-2699; Ohishi, et al., 2002, J. Clin. Invest. 110:1165-1174; Delaney, et al., 2010, Nature Med. 16(2): 232-236; and Chapter 2 of Regenerative Medicine, Department of Health and Human Services, August 2006, and the references cited therein. The collection and processing of other cell types described herein are known by one of ordinary skill in the art.

In particular embodiments, the isolating, incubating, expansion, and/or engineering steps are carried out in a sterile or contained environment and/or in an automated fashion, such as controlled by a computer attached to a device in which the steps are performed. Final formulation of modified cells into formulations for administration to subjects are known to those of ordinary skill in the art, and relevant aspects of these processes are described elsewhere herein.

Targeted viral vectors and/or nanoparticles can also be used to genetically-modify immune cells in vivo. Viral vectors that can be used to deliver fusion protein-encoding genes to cells and numerous targeted (e.g., pseudotyped) viral vectors are known to those of ordinary skill in the art.

Exemplary cell-targeted nanoparticles include a cell targeting ligand (e.g., CD3, CD4, CD8, CD34) on the surface of the nanoparticle wherein the cell targeting ligand results in selective uptake of the nanoparticle by a selected cell type. The nanoparticle then delivers gene modifying components that result in expression of the DARIC.

Exemplary nanoparticles include liposomes (microscopic vesicles including at least one concentric lipid bilayer surrounding an aqueous core), liposomal nanoparticles (a liposome structure used to encapsulate another smaller nanoparticle within its core); and lipid nanoparticles (liposome-like structures that lack the continuous lipid bilayer characteristic of liposomes). Other polymer-based nanoparticles can also be used as well as porous nanoparticles constructed from any material capable of forming a porous network. Exemplary materials include metals, transition metals and metalloids (e.g., lithium, magnesium, zinc, aluminum and silica).

For in vivo delivery and cellular uptake, nanoparticles can have a neutral or negatively-charged coating and a size of 130 nm or less. Dimensions of the nanoparticles can be determined using, e.g., conventional techniques, such as dynamic light scattering and/or electron microscopy.

### (vi) FORMULATIONS

Formulations described herein can include ex vivo modified cells, vectors for ex vivo or in vivo transduction, or dimerization agents such as rapamycin and/or analogs thereof.

A “pharmaceutical” formulation or composition includes an active compound for administration (e.g., a genetically modified cell, viral vector, nanoparticle, drug molecule, or dimerizing agent) within a pharmaceutically-acceptable carrier.

The phrase “pharmaceutically acceptable” refers to those compounds, materials, and/or dosage forms which are, within the scope of sound medical judgment, suitable for use in contact with the tissues of human beings and animals without excessive toxicity, commensurate with a reasonable benefit/risk ratio. In certain instances, pharmaceutically-acceptable carriers have been approved by a relevant regulatory agency (e.g., the United States Food and Drug Administration (US FDA)).

Depending on the context and active compound (or active ingredient) for delivery, “pharmaceutically acceptable carriers” include any adjuvant, excipient, glidant, diluent, preservative, dye/colorant, surfactant, wetting agent, dispersing agent, suspending agent, stabilizer, isotonic agent, solvent, surfactant, or emulsifier which meets the requirements noted above. Exemplary pharmaceutically acceptable carriers are disclosed in Remington's Pharmaceutical Sciences, 18th Ed. Mack Printing Company, 1990. Moreover, formulations and compositions can be prepared to meet sterility, pyrogenicity, general safety, and purity standards as required by the US FDA Office of Biological Standards and/or other relevant foreign regulatory agencies.

Exemplary pharmaceutically-acceptable carriers include saline, buffered saline, physiological saline, water, Hanks' solution, Ringer's solution, Nonnosol-R (Abbott Labs), PLASMA-LYTE A® (Baxter Laboratories, Inc., Morton Grove, IL), glycerol, ethanol, and combinations thereof. In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Pharmaceutically acceptable carriers for therapeutic use are also well known in the pharmaceutical art, and are described, for example, in the Physicians Desk Reference, 62nd edition. Oradell, NJ: Medical Economics Co., 2008; Goodman & Gilman's The Pharmacological Basis of Therapeutics, Eleventh Edition. McGraw-Hill, 2005; Remington: The Science and Practice of Pharmacy, 20th Edition. Baltimore, MD: Lippincott Williams & Wilkins, 2000; and The Merck Index, Fourteenth Edition. Whitehouse Station, NJ: Merck Research Laboratories, 2006.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols, amino acids, organic sugars or sugar alcohols, PEG, sulfur-containing reducing agents, bovine serum albumin, gelatin or immunoglobulins, polyvinylpyrrolidone, and saccharides.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens, catechol, resorcinol, cyclohexanol, and 3-pentanol.

Therapeutically effective amounts of active ingredients within formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts of dimerizing agents within formulations per body area of subject can range from 0.1 to 5 mg or mg/m² or from 0.5 to 1 mg or mg/m². In other examples, a dose can include 0.1 mg/m², 0.3 mg/m², 0.75 mg, 0.9 mg, 1.5 mg, 0.1 to 5 mg or mg/m² or from 0.5 to 1 mg or mg/m². In other examples, a dose can include 0.1 mg/m², 0.2 mg/m², 0.3 mg/m², 0.4 mg/m², 0.5 mg/m², 0.6 mg/m², 0.7 mg/m², 0.75 mg or mg/m², 0.8 mg, 0.9 mg, 1 mg or more. In particular embodiments, therapeutically effective amounts of dimerizing agents within formulation include 0.75 mg-5.0 mg for subjects greater than 1.5 m². In particular embodiments, therapeutically effective amounts of dimerizing agents within formulation include less than 0.75 mg/m2 (e.g., 0.50 mg/m²) for subjects less than or equal to 1.5 m².

Therapeutically effective amounts of dimerizing agents within formulations should be such that the dose maintains a target trough blood level of 1-4 ng/mL (e.g., 1.5-3 ng/mL) of dimerizing agent per blood volume. In other examples, a dose can result in target trough blood level of 1 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, 2.0 ng/mL, 2.1 ng/mL, 2.2 ng/mL, 2.3 ng/mL, 2.4 ng/mL, 2.5 ng/mL, 2.6 ng/mL, 2.7 ng/mL, 2.8 ng/mL, 2.9 ng/mL, or 3.0 ng/mL. In other examples, a dose can result in target trough blood level ranging from 1 ng/mL-5 ng/mL. In particular embodiments, therapeutically effective amounts of dimerizing agents within formulations should be such that the dose maintains a target trough blood level of 2 ng/mL of dimerizing agent per blood volume. In particular embodiments, therapeutically effective amounts of dimerizing agents within formulations should be such that the dose maintains a target trough blood level of 1.5-3 ng/mL of dimerizing agent per blood volume.

In particular embodiments, administration of formulations results in the multimerization of a first fusion protein and a second fusion protein such that the dimerizing agent binds a multimerization domain of the first fusion protein and a multimerization domain of the second fusion protein thereby forming a DARIC.

Formulations and compositions can be prepared for administration by, e.g., injection, infusion, perfusion, lavage, or ingestion. The formulations and compositions can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection. Particular embodiments utilize oral liquid formulations and/or solid tablets for the administration of dimerizing agents.

### (vii) THERAPEUTIC METHODS

In particular embodiments, the present disclosure provides a method for modulating immune cell activation, including administering to a subject in need thereof an effective amount of cells expressing DARIC, agents to cause expression of DARIC in cells, or pharmaceutical compositions thereof, wherein the DARIC includes a first fusion protein including a first multimerization domain; and a second fusion protein including a second fusion domain and an intracellular component; and administering a dimerizing agent that binds both the first multimerization domain and the second multimerization domain thereby priming the DARIC for signaling. In particular embodiments the first fusion protein further includes a binding domain that binds a target cell antigen. When the binding domain of the DARIC binds the target cell antigen, the immune cell is activated.

In other embodiments, the first fusion protein does not includes a binding domain, and further includes an intracellular component. When the dimerizing agent that binds both the first multimerization domain and the second multimerization domain the DARIC is primed for signaling and may signal.

The term “administer”, “administering”, or “administration” refers to the dispensing or applying treatment.

The present disclosure provides methods and compositions for priming a DARIC for signaling by inducing the multimerization of at least a first fusion protein and a second fusion protein to thereby form the DARIC. More particularly, the disclosure relates to modulating the multimerization of a first fusion protein including an FKBP-rapamycin binding multimerization domain and a second fusion protein including an FK506 binding protein multimerization domain using rapamycin or analogs thereof for the formation of a DARIC that is primed for signaling.

In another aspect, the present disclosure provides a method for inhibiting growth, metastasis or metastatic growth of a malignancy (e.g., a solid malignancy or a hematologic malignancy), including administering to a subject in need thereof an effective amount of a cell encoding a DARIC and a dimerizing agent provided herein or a composition thereof.

A “subject in need” refers to a subject at risk of, or suffering from, a disease, disorder or condition that is amenable to treatment or amelioration with a non-natural cell, polypeptide complex or a composition thereof provided herein. In certain embodiments, a subject is a human. In particular embodiments, the subject is a pediatric patient. In particular embodiments, the subject is no more than 18 years old. In particular embodiments, the subject is at least 18 years old. In particular embodiments, the subject is a late adolescent, typically defined as 18-21 years old. In particular embodiments, the subject is 18, 19, 20, 21, 22, 23, 23, 25, 26, 27, or 28 years old. In particular embodiments, the subject is 16-30 years old. In additional embodiments, the subject is ≤31 yo, ≤30 yo, or ≤26 yo. In additional embodiments, the subject is an adult (greater than 31 years old).

In particular embodiments, the subject has or is diagnosed with a cancer, infectious disease, autoimmune disease, inflammatory disease, and immunodeficiency or condition associated therewith.

A wide variety of cancers, including solid malignancy and hematologic malignancy, are amenable to the compositions and methods disclosed herein. Types of cancer that may be treated include adenocarcinoma of the breast, prostate, pancreas, colon and rectum; all forms of bronchogenic carcinoma of the lung (including squamous cell carcinoma, adenocarcinoma, small cell lung cancer and non-small cell lung cancer); myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional types of cancers that may be treated include: histiocytic disorders; leukemia; histiocytosis malignant; Hodgkin's disease; non-Hodgkin's lymphoma; plasmacytoma; reticuloendotheliosis; melanoma; renal cell carcinoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor.

In particular embodiments, the subject has or is diagnosed with solid cancer. In particular embodiments, the solid cancer is selected from the group including: lung cancer (e.g., non-small cell lung carcinoma), squamous cell carcinoma (e.g., head and neck squamous cell carcinoma), colorectal cancer, pancreatic cancer, breast cancer, thyroid cancer, bladder cancer, cervical cancer, esophageal cancer, ovarian cancer, gastric cancer endometrial cancer, or brain cancer (e.g., gliomas, glioblastomas, or oligodendrogliomas).

“Treatment,” “treating” or “treated” refers to either a therapeutic treatment or prophylactic/preventative treatment. A treatment is therapeutic if at least one symptom of disease in an individual receiving treatment improves or a treatment may delay worsening of a progressive disease in an individual, or prevent onset of additional associated diseases.

Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin; and glioblastoma multiforme. The types of cancers that may be treated also include angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Additional exemplary cancers that are also amenable to the compositions and methods disclosed herein are B-cell cancers, including B-cell lymphomas [such as various forms of Hodgkin's disease, non-Hodgkins lymphoma (NHL) or central nervous system lymphomas], leukemias [such as acute lymphoblastic leukemia (ALL), chronic lymphocytic leukemia (CLL), Hairy cell leukemia and chronic myoblastic leukemia] and myelomas (such as multiple myeloma). Additional B cell cancers include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt lymphoma/leukemia, B-cell proliferations of uncertain malignant potential, lymphomatoid granulomatosis, and post-transplant lymphoproliferative disorder.

In particular embodiments, the subject has or is diagnosed with a hematological malignancy. In particular embodiments, the hematological malignancy is a leukemia, lymphoma, or multiple myeloma. In particular embodiments, the hematological malignancy is acute myelogenous leukemia (AML).

In another aspect, the present disclosure provides a method for treating an autoimmune or inflammatory disease, disorder or condition, including administering to a subject in need thereof an effective amount of a cell including DARIC and a dimerizing agent as described herein or a composition thereof.

Exemplary autoimmune or inflammatory diseases, disorders or conditions that may be treated by the fusion proteins and compositions and unit dose forms thereof include inflammatory bowel disease (e.g., Crohn's disease or ulcerative colitis), diabetes mellitus (e.g., type I diabetes), dermatomyositis, polymyositis, pernicious anaemia, primary biliary cirrhosis, acute disseminated encephalomyelitis (ADEM), Addison's disease, ankylosing spondylitis, antiphospholipid antibody syndrome (APS), autoimmune hepatitis, Goodpasture's syndrome, Graves' disease, Guillain-Barre syndrome (GBS), Hashimoto's disease, idiopathic thrombocytopenic purpura, systemic lupus erythematosus, lupus nephritis, neuropsychiatric lupus, multiple sclerosis (MS), myasthenia gravis, pemphigus vulgaris, asthma, psoriatic arthritis, rheumatoid arthritis, Sjögren's syndrome, temporal arteritis (also known as “giant cell arteritis”), autoimmune hemolytic anemia, Bullous pemphigoid, vasculitis, coeliac disease, chronic obstructive pulmonary disease, endometriosis, Hidradenitis suppurativa, interstitial cystitis, morphea, scleroderma, narcolepsy, neuromyotonia, vitiligo, and autoimmune inner ear disease.

In certain embodiments, a method for treating a hyperproliferative, inflammatory, autoimmune, or graft-versus-host disease, includes (a) administering an engineered cell including a first and a second nucleic acid molecule, wherein the first nucleic acid molecule encodes a first fusion protein including a first multimerization domain, a transmembrane domain, and an intracellular component, and the second nucleic acid molecule encodes a second fusion protein including a binding domain and a second multimerization domain; and (c) administering a dimerizing agent, wherein the dimerizing agent promotes the formation of a DARIC on the engineered cell surface with the dimerizing agent associated with and disposed between the multimerization domains of the first and second fusion proteins; wherein the binding domain of the DARIC specifically binds a cell surface target on a hyperproliferative, inflammatory, autoimmune, or graft-versus-host disease cell to promote an immunomodulatory response and thereby treats the disease.

In particular embodiments, a method for treating a hyperproliferative, inflammatory, autoimmune, or graft-versus-host disease, includes (a) administering one or more engineered cells including a first nucleic acid molecule and a second nucleic acid molecule, wherein the first nucleic acid molecule encodes a first fusion protein including a first multimerization domain, and the second nucleic acid molecule encodes a second fusion protein including a second multimerization domain, and (c) administering a dimerizing agent, wherein the dimerizing agent promotes the formation of a DARIC primed for signaling, e.g., a BiTE, with the dimerizing agent associated with and disposed between the multimerization domains of the first and second fusion proteins; wherein the binding domain of the DARIC specifically binds a cell surface target on a hyperproliferative, inflammatory, autoimmune, or graft-versus-host disease cell to promote an immunomodulatory response and thereby treats the disease.

In particular embodiments, cells are genetically modified to express the components of a DARIC. In particular embodiments, cells are genetically modified to express a first fusion protein including a first multimerization domain, and a second fusion protein including a second multimerization domain. In particular embodiments, the cells can be genetically modified in vivo or ex vivo. In particular embodiments, genetically modified cells are administered to a subject at a cell dose per weight of subject ranges from 1×10⁵ cells/kg to 2000×10⁶ cells/kg, 1×10⁶ cells/kg to 1000×10⁶ cells/kg, 1×10⁶ cells/kg to 100×10⁶ cells/kg, 5×10⁶ cells/kg to 500×10⁶ cells/kg, 10×10⁶ cells/kg to 1000×10⁶ cells/kg, 1×10⁶ cells/kg to 2×10⁶ cells/kg, 3×10⁶ cells/kg to 5×10⁶ cells/kg, or 7.5×10⁶ cells/kg to 10×10⁶ cells/kg.

In particular embodiments, the subject is lymphodepleted prior to administration of the genetically modified cells or prior to genetically modifying the cells. In particular embodiments, lymphodepletion includes administering Fludarabine 30 mg/m² IV once daily for 4 days; and Cyclophosphamide 500 mg/m² IV once daily for 2 days. In particular embodiments, Cyclophosphamide is administered on days 3 and 4 of Fludarabine administration. In particular embodiments, lymphodepletion begins 5 days prior to administering the cells or genetically modifying the cells.

When administration of a dose is described in grams per m², the amount of dose is calculated based on the body area of the subject. For example, a dose of 0.50 mg/m² means that a dose of 0.50 mg would be administered to a patient that is 1 m² in body area.

In particular embodiments, dimerizing agent is administered orally. Dimerizing agents can be administered orally as, for example, a liquid or as a solid (e.g., a solid tablet). In particular embodiments, dimerizing agent is orally administered at a dose of 0.75 mg, 1.0 mg, 1.25 mg, 1.5 mg, 1.75 mg, 2.0 mg, 2.25 mg, 2.5 mg, 2.75 mg, 3.0 mg, 3.25 mg, 3.5 mg, 3.75 mg, or 4 mg for subjects greater than 1.5 m². In particular embodiments, dimerizing agent is orally administered at a dose of 0.75 mg or 3.0 mg for subjects greater than 1.5 m². In particular embodiments, dimerizing agent is orally administered at a dose of at least 0.75 mg for subjects greater than 1.5 m². In particular embodiments, dimerizing agent is orally administered at a dose of at least 1 mg for subjects greater than 1.5 m². In particular embodiments, dimerizing agent is orally administered at a dose of at least 1.25 mg for subjects greater than 1.5 m². In particular embodiments, dimerizing agent is orally administered at a dose of at least 1.5 mg for subjects greater than 1.5 m². In particular embodiments, dimerizing agent is orally administered at a dose of at least 1.75 mg for subjects greater than 1.5 m². In particular embodiments, dimerizing agent is orally administered at a dose of at least 2 mg for subjects greater than 1.5 m². In particular embodiments, dimerizing agent is orally administered at a dose of at least 2.25 mg for subjects greater than 1.5 m². In particular embodiments, dimerizing agent is orally administered at a dose of at least 2.5 mg for subjects greater than 1.5 m². In particular embodiments, dimerizing agent is orally administered at a dose of at least 2.75 mg for subjects greater than 1.5 m². In particular embodiments, dimerizing agent is orally administered at a dose of 3.0 mg for subjects greater than 1.5 m².

In particular embodiments, dimerizing agent is administered orally at a dose of 0.50 mg/m² for subjects less than or equal to 1.5 m². In particular embodiments, dimerizing agent is administered orally at a dose of 0.1-2.0 mg/m² for subjects less than or equal to 1.5 m². In particular embodiments, the dosing should be adjusted to maintain a target trough blood level of 2 ng/mL. In various embodiments, dosing should be adjusted to maintain a target trough blood level within a target range of 1-4 ng/mL. In particular embodiments, dosing should be adjusted to maintain a target trough blood level within a target range of 1.5-3 ng/mL. In particular embodiments, dosing should be adjusted to maintain a target trough blood level within a target range of 3-9 ng/mL. In particular embodiments, dosing should be adjusted to maintain a target trough blood level within a target range of 1-2 ng/mL.

In particular embodiments, rapamycin or an analog thereof is administered orally at a dose of 0.75 mg or greater for subjects greater than 1.5 m². In particular embodiments, rapamycin or an analog thereof is administered orally at a dose of 0.50 mg/m² for subjects less than or equal to 1.5 m². In particular embodiments, rapamycin or an analog thereof is administered orally at a dose of 0.1-2.0 mg/m². In particular embodiments, dosing should be adjusted to maintain a target trough blood level of 2 ng/mL. In particular embodiments, dosing should be adjusted to maintain a target trough blood level within a target range of 1.5-3 ng/mL. In particular embodiments, dosing should be adjusted to maintain a target trough blood level within a target range of 3-9 ng/mL. In particular embodiments, dosing should be adjusted to maintain a target trough blood level within a target range of 1-2 ng/mL.

In particular embodiments, administration of dimerizing agent begins on day 2 after engineered cell product infusion. In particular embodiments, administration of dimerizing agent begins on day 1 after engineered cell product infusion. In particular embodiments, administration of dimerizing agent begins on day 3 after engineered cell product infusion. In particular embodiments, administration of dimerizing agent begins on day 4 after engineered cell product infusion. In particular embodiments, dimerizing agent is administered daily, once administration begins. In particular embodiments, dimerizing agent is administered twice a day. In particular embodiments, dimerizing agent will be administered daily from day 2 to day 21 (or day 3 to day 21) after engineered cell product infusion. In particular embodiments, dimerizing agent will be administered daily for 10, 11, 12, 13, 14, 15, 16, 17, 18, 19, 20, 21, 22, 23, 24, 25, 26, 27, 28, 29, or 30 days, once administration begins. In particular embodiments, dimerizing agent will be administered at least 16 hours after a dose of cells. In particular embodiments, dimerizing agent will be administered at least 24 hours, at least 36 hours, at least 48 hours, at least 60 hours, at least 72 hours, or at least 84 hours post administering the cells. In particular embodiments, dimerizing agent will be administered from 1 to 4, 1 to 3, 1 to 2, 2 to 4, 3 to 4, or 2 to 3 days post administering the dose of cells. In particular embodiments, administration of the dimerizing agent includes a rest period before administration of subsequent dimerizing agent courses. In particular embodiments, the rest period is at least 10 to 45 days.

In particular embodiments, rapamycin or an analog thereof is administered on day 2 after engineered cell product infusion. In particular embodiments, rapamycin or an analog thereof is administered on day 1 after engineered cell product infusion. In particular embodiments, rapamycin or an analog thereof is administered on day 3 after engineered cell product infusion. In particular embodiments, rapamycin or an analog thereof is administered on day 4 after engineered cell product infusion. In particular embodiments, rapamycin or an analog thereof is administered daily. In particular embodiments, rapamycin or an analog thereof is administered twice a day. In particular embodiments, rapamycin or an analog thereof will be administered daily from day 2 to day 21 after engineered cell product infusion. In particular embodiments, rapamycin or an analog thereof will be administered for 10, 11, 12, 13, 14, 15, 16, 17, 18, 19, 20, 21, 22, 23, 24, 25, 26, 27, 28, 29, or 30 days. In particular embodiments, dimerizing agent will be administered simultaneously with the dose of cells. In particular embodiments, rapamycin or an analog thereof will be administered the same day as the dose of cells. In particular embodiments, rapamycin or an analog thereof will be administered at least 24 hours, at least 36 hours, at least 48 hours, at least 60 hours, at least 72 hours, or at least 84 hours post administering the cells. In particular embodiments, rapamycin or an analog thereof will be administered from 1 to 4, 1 to 3, 1 to 2, 2 to 4, 3 to 4, or 2 to 3 days post administering the dose of cells. In particular embodiments, administration of the rapamycin or an analog thereof includes a rest period before administration of subsequent course of rapamycin or analog thereof. In particular embodiments, the rest period is at least 10 to 45 days.

A “course”, as it refers to treatment or therapy, refers to the administration of a drug, compound, or therapy in one or more separate administrations. For example, a course may include a dose of a drug, compound, or therapy once a day for 10 days. After a rest period, another course of treatment can be administered.

In particular embodiments, a bone marrow aspirate and/or biopsy will be conducted and analyzed for disease. In particular embodiments, the bone marrow aspirate and/or biopsy will be conducted on day 28. In particular embodiments, dimerizing agent will continue to be withheld in a subject in morphological remission with <1% disease by multiparameter flow. In particular embodiments, subsequent dimerizing agent courses can be administered in subjects with evidence of persistent disease at a level of >1% in the bone marrow. In particular embodiments, disease is leukemia. In particular embodiments, disease includes leukemia. In particular embodiments, subjects in remission are administered subsequent dimerizing agent courses. In particular embodiments, subjects with an absence of Grade 3 or higher toxicity are administered subsequent dimerizing agent courses. In particular embodiments, subjects in remission with an absolute phagocyte count (APC) greater than 500 cells/μL are administered subsequent dimerizing agent courses. In particular embodiments, subjects in remission with an absolute phagocyte count (APC) greater than 400 cells/μL are administered subsequent dimerizing agent courses. In particular embodiments, subjects in remission with an absolute phagocyte count (APC) greater than 300 cells/μL are administered subsequent dimerizing agent courses. In particular embodiments, subsequent dimerizing agent courses are administered at day 42 after cells modified to express DARIC are infused into subject. In particular embodiments, subsequent dimerizing agent courses are administered at day 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 after cells modified to express DARIC are infused into subject. In particular embodiments, subsequent dimerizing agent courses are administered at any day after cells modified to express DARIC are infused into subject. In particular embodiment, subsequent dimerizing agent courses are administered 14 days after cessation of prior dimerizing agent administration. In particular embodiment, subsequent dimerizing agent courses are administered 7, 8, 9, 10, 11, 12, 13, 14, 15, 16, 17, 18, 19, 20, 21, 22, 23, 24, 25, 26 or more days after cessation of prior dimerizing agent administration.

In particular embodiments, a bone marrow aspirate and/or biopsy will be conducted and analyzed for disease. In particular embodiments, the bone marrow aspirate and/or biopsy will be conducted on day 28. In particular embodiments, rapamycin or an analog thereof will continue to be withheld in a subject in morphological remission with <1% disease by multiparameter flow. In particular embodiments, subsequent courses of rapamycin or an analog thereof can be administered in subjects with evidence of persistent disease at a level of >1% in the bone marrow. In particular embodiments, disease is leukemia. In particular embodiments, disease includes leukemia. In particular embodiments, subjects in remission are administered subsequent courses of rapamycin or analog thereof. In particular embodiments, subjects with an absence of Grade 3 or higher toxicity are administered subsequent courses of rapamycin or analog thereof. In particular embodiments, subjects in remission with an absolute phagocyte count (APC) greater than 500 cells/μL are administered subsequent courses of rapamycin or an analog thereof. In particular embodiments, subjects in remission with an absolute phagocyte count (APC) greater than 400 cells/μL are administered subsequent courses of rapamycin or analog thereof. In particular embodiments, subjects in remission with an absolute phagocyte count (APC) greater than 300 cells/μL are administered subsequent courses of rapamycin or analog thereof. In particular embodiments, subsequent courses of rapamycin or an analog thereof are administered at day 42 after cells modified to express DARIC are infused into subject. In particular embodiments, subsequent courses of rapamycin or an analog thereof are administered at day 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 after cells modified to express DARIC are infused into subject. In particular embodiments, subsequent courses of rapamycin or an analog thereof are administered at any day after cells modified to express DARIC are infused into subject. In particular embodiment, subsequent courses of rapamycin or an analog thereof are administered 14 days after cessation of prior rapamycin or an analog thereof administration. In particular embodiment, subsequent courses of rapamycin or an analog thereof are administered 7, 8, 9, 10, 11, 12, 13, 14, 15, 16, 17, 18, 19, 20, 21, 22, 23, 24, 25, 26 or more days after cessation of prior rapamycin or an analog thereof administration.

Any of the aforementioned non-natural cells, fusion proteins, dimerizing agents and other accessory molecules may be used in the methods of treatment of this disclosure.

### (viii) EXEMPLARY EMBODIMENTS

### Exemplary Embodiments—Set 1

1. A method including
Identifying a subject with cells expressing a dimerizing agent regulated immunomodulatory complex (DARIC), wherein the DARIC includes:
   a first fusion protein including a CD33 single domain variable heavy (VHH) binding domain, an FK506 binding protein (FKBP) multimerization domain, and a transmembrane domain, and
   a second fusion protein including an FKBP-rapamycin binding (FRB) multimerization domain, a transmembrane domain, and an intracellular component; and
administering to the subject a course of rapamycin or an analog thereof that binds and is disposed between the multimerization domain of the first fusion protein and the multimerization domain of the second fusion protein
wherein the course:
   results in a blood trough level of the rapamycin or the analog thereof of 1.5 ng/mL to 3 ng/mL;
   begins 2 or 3 days after the subject has in vivo cells expressing the DARIC;
   extends for 18, 19, or 20 days with daily administrations of the rapamycin or the analog thereof;
   includes a daily dose of 0.75 mg or more if the subject is greater than 1.5 m² or a daily dose of less than 0.75 mg if the subject is 1.5 m² or less; and
   includes a rest period at the end of the course wherein no rapamycin or analog thereof is administered to the subject.

2. A method including
Identifying a subject with cells expressing a dimerizing agent regulated immunomodulatory complex (DARIC), wherein the DARIC includes:
   a first fusion protein including an extracellular component, an FK506 binding protein (FKBP) or FKBP-rapamycin binding (FRB) multimerization domain or variant thereof, and a transmembrane domain, and
   a second fusion protein including an FK506 binding protein (FKBP) or FKBP-rapamycin binding (FRB) multimerization domain or variant thereof, a transmembrane domain, and an intracellular component; and
administering to the subject a course of rapamycin or an analog thereof that binds and is disposed between the multimerization domain of the first fusion protein and the multimerization domain of the second fusion protein
wherein the course one or more of:
   results in a blood trough level of the rapamycin or the analog thereof of 1.5 ng/mL to 3 ng/mL;
   begins 0-4 days after the subject has in vivo cells expressing the DARIC;
   extends for at least 14 days with daily administrations of the rapamycin or the analog thereof;
   includes a rest period at the end of the course wherein no rapamycin or analog thereof is administered to the subject.

3. A method including
Identifying a subject with cells expressing a dimerizing agent regulated immunomodulatory complex (DARIC), wherein the DARIC includes:
   a first fusion protein including an extracellular component, an FK506 binding protein (FKBP) or FKBP-rapamycin binding (FRB) multimerization domain or variant thereof, and a transmembrane domain, and
   a second fusion protein including an FK506 binding protein (FKBP) or FKBP-rapamycin binding (FRB) multimerization domain or variant thereof, a transmembrane domain, and an intracellular component; and
administering to the subject a course of rapamycin or an analog thereof that binds and is disposed between the multimerization domain of the first fusion protein and the multimerization domain of the second fusion protein
wherein the course one or more of:
   results in a blood trough level of the rapamycin or the analog thereof of 1.5 ng/mL to 3 ng/mL;
   includes a daily dose of 0.75 mg or more if the subject is greater than 1.5 m² or a daily dose of less than 0.75 mg, if the subject is 1.5 m² or less;
   and
   includes a rest period at the end of the course wherein no rapamycin or analog thereof is administered to the subject.

4. A method including
Identifying a subject with cells expressing a dimerizing agent regulated immunomodulatory complex (DARIC), wherein the DARIC includes:
   a first fusion protein including a multimerization domain, and
   a second fusion protein including a multimerization domain; and administering to the subject a course of rapamycin or an analog thereof that binds and is disposed between the multimerization domain of the first fusion protein and the multimerization domain of the second fusion protein.

5. The method of embodiment 4, wherein the course results in a blood trough level of the rapamycin or the analog thereof of 1.0 ng/mL to 3 ng/mL.

6. The method of embodiment 4, wherein the course begins 0-4 days after the subject has in vivo cells expressing the DARIC.

7. The method of any of embodiments 4-6, wherein the course begins 2 or 3 days after the subject has in vivo cells expressing the DARIC.

8. The method of any of embodiments 4-7, wherein the course extends for at least 14 days.

9. The method of any of embodiments 4-8, wherein the course extends for 18, 19, 20, 21, or 22 days with daily administrations of the rapamycin or the analog thereof.

10. The method of any of embodiments 4-9, wherein the course includes a daily dose of at least 0.75 mg if the subject has a body surface area of greater than 1.5 m² or a daily dose of less than 0.75 mg if the subject has a body surface area of 1.5 m² or less.

11. The method of embodiment 10, wherein the subject has a body surface area of greater than 1.5 m² and the daily dose is 0.75 mg-3.5 mg.

12. The method of embodiment 10, wherein the subject has a body surface area of 1.5 m² or less and the daily dose is 0.25 mg/m²-0.74 mg/m².

13. The method of embodiment 10, wherein the subject has a body surface area of 1.5 m² or less and the daily dose is 0.50 mg/m².

14. The method of embodiment 10, wherein the course includes a rest period wherein no rapamycin or analog thereof is administered to the subject after the course of daily administrations.

15. The method of embodiment 14, wherein the rest period is 13, 14, or 15 days.

16. The method of embodiment 14, wherein the rest period is at least 13 days, at least 14 days, at least 15 days, at least 16 days, at least 17 days, at least 18 days, at least 19 days, at least 20 days, at least 21 days, or at least 22 days.

17. The method of embodiment 14, wherein the rest period is at least 14 days.

18. The method of embodiment 14, wherein the rest period is 14 days.

19. The method of embodiment 14, wherein the method further includes administering a second course of the rapamycin or analog thereof after the rest period.

20. The method of embodiment 19, wherein the second course results in a blood trough level of the rapamycin or the analog thereof of 1.0 ng/mL to 3 ng/mL.

21. The method of embodiment 19 or 20, wherein the second course extends for at least 14 days.

22. The method of embodiment 19 or 20, wherein the second course extends for 18, 19, 20, 21, or 22 days with daily administrations of the rapamycin or the analog thereof.

23. The method of any of embodiments 19-22, wherein the second course includes a daily dose of at least 0.75 mg if the subject has a body surface area of greater than 1.5 m² or a daily dose of less than 0.75 mg if the subject has a body surface area of 1.5 m² or less.

24. The method of any of embodiments 4-23, wherein
the multimerization domain of the first fusion protein includes an FKBP-rapamycin binding (FRB) multimerization domain or variant thereof, and the multimerization domain of the second fusion protein includes an FK506 binding protein (FKBP) multimerization domain or variant thereof or
the multimerization domain of the first fusion protein includes an FK506 binding protein (FKBP) multimerization domain or a variant thereof, and the multimerization domain of the second fusion protein includes an FKBP-rapamycin binding (FRB) multimerization domain or a variant thereof.

25. The method of embodiment 24, wherein the FKBP multimerization domain or the variant thereof is FKBP12.

26. The method of embodiment 24, wherein the FKBP multimerization domain or the variant thereof has the sequence as set forth in SEQ ID NO: 55 or 56 or has at least 90% sequence identity to the sequence as set forth in SEQ ID NO: 55 or 56.

27. The method of embodiment 24, wherein the FKBP multimerization domain or the variant thereof has the sequence as set forth in SEQ ID NO: 55 or 56 or has at least 95% sequence identity to the sequence as set forth in SEQ ID NO: 55 or 56.

28. The method of embodiment 24, wherein the FKBP multimerization domain or the variant thereof has the sequence as set forth in SEQ ID NO: 55 or 56 or has at least 98% sequence identity to the sequence as set forth in SEQ ID NO: 55 or 56.

29. The method of embodiment 24, wherein the FRB multimerization domain or the variant thereof is FRB T2098L.

30. The method of embodiment 24, wherein the FRB multimerization domain or the variant thereof has the sequence as set forth in SEQ ID NO: 57 or 58 or has at least 90% sequence identity to the sequence as set forth in SEQ ID NO: 57 or 58.

31. The method of embodiment 24, wherein the FRB multimerization domain or the variant thereof has the sequence as set forth in SEQ ID NO: 57 or 58 or has at least 95% sequence identity to the sequence as set forth in SEQ ID NO: 57 or 58.

32. The method of embodiment 24, wherein the FRB multimerization domain or the variant thereof has the sequence as set forth in SEQ ID NO: 57 or 58 or has at least 98% sequence identity to the sequence as set forth in SEQ ID NO: 57 or 58.

33. The method of embodiment 24, wherein the FRB multimerization domain and the FKBP multimerization domain localize extracellularly when the first fusion protein and the second fusion protein are expressed.

34. The method of embodiment 24, wherein the FRB multimerization domain and the FKBP multimerization domain localize intracellularly when the first fusion protein and the second fusion protein are expressed.

35. The method of any of embodiments 4-34, wherein the first fusion protein and/or the second fusion protein further include a binding domain.

36. The method of embodiment 35, wherein the binding domain binds a cancer antigen.

37. The method of embodiment 35 or 36, wherein the binding domain is a single domain variable heavy chain (VHH) or a single chain variable fragment (scFv).

38. The method of embodiment 35 or 36, wherein the binding domain includes a receptor extracellular domain or ligand.

39. The method of any of embodiments 35-38, wherein the binding domain includes a binding domain of a CD33 antibody.

40. The method of embodiment 39, wherein the binding domain of the CD33 antibody is a VHH.

41. The method of embodiment 40, wherein the VHH has the sequence as set forth in any one of SEQ ID NOs: 2, 3, 4, 5, 6, 7, 8, 9, 10, 11, 12, 13, 14, 15, 16, 17, 18, 19, 20, or 21.

42. The method of embodiment 40, wherein the VHH has at least 90% sequence identity to the sequence as set forth in any one of SEQ ID NOs: 2, 3, 4, 5, 6, 7, 8, 9, 10, 11, 12, 13, 14, 15, 16, 17, 18, 19, 20, or 21 and specifically binds CD33.

43. The method of embodiment 40, wherein the VHH has at least 95% sequence identity to the sequence as set forth in any one of SEQ ID NOs: 2, 3, 4, 5, 6, 7, 8, 9, 10, 11, 12, 13, 14, 15, 16, 17, 18, 19, 20, or 21 and specifically binds CD33.

44. The method of embodiment 40, wherein the VHH has at least 98% sequence identity to the sequence as set forth in any one of SEQ ID NOs: 2, 3, 4, 5, 6, 7, 8, 9, 10, 11, 12, 13, 14, 15, 16, 17, 18, 19, 20, or 21 and specifically binds CD33.

45. The method of any of embodiments 4-44, wherein the first fusion protein and/or the second fusion protein include a binding domain that binds CLL1.

46. The method of embodiment 45, wherein the binding domain that binds CLL1 has the sequence as set forth in any one of SEQ ID NOs: 43, 44, 45, 46, 47, 48, 49, 50, 51, 52, 53 or 54.

47. The method of embodiment 45, wherein the binding domain that binds CLL1 has at least 90% sequence identity to the sequence as set forth in any one of SEQ ID NOs: 43, 44, 45, 46, 47, 48, 49, 50, 51, 52, 53 or 54 and specifically binds CLL1.

48. The method of embodiment 45, wherein the binding domain that binds CLL1 has at least 95% sequence identity to the sequence as set forth in any one of SEQ ID NOs: 43, 44, 45, 46, 47, 48, 49, 50, 51, 52, 53 or 54 and specifically binds CLL1.

49. The method of embodiment 45, wherein the binding domain that binds CLL1 has at least 98% sequence identity to the sequence as set forth in any one of SEQ ID NOs: 43, 44, 45, 46, 47, 48, 49, 50, 51, 52, 53 or 54 and specifically binds CLL1.

50. The method of any of embodiments 4-49, wherein the first fusion protein and/or the second fusion protein further include an intracellular component.

51. The method of embodiment 50, wherein the intracellular component includes an intracellular primary signaling domain.

52. The method of embodiment 51, wherein the intracellular primary signaling domain includes CD3ζ or a fragment thereof.

53. The method of embodiment 50 or 51, wherein the intracellular component includes a co-receptor domain.

54. The method of any of embodiments 50-53, wherein the intracellular component includes a costimulatory domain.

55. The method of embodiment 54, wherein the costimulatory domain includes Toll-like receptor 1 (TLR1), TLR2, TLR3, TLR4, TLR5, TLR6, TLR7, TLR8, TLR9, TLR10, caspase recruitment domain family member 11 (CARD11), CD2, CD7, CD27, CD28, CD30, CD40, CD54 (ICAM), CD83, CD94, CD134 (OX40), CD137 (4-1BB), CD278 (ICOS), DNAX-Activation Protein 10 (DAP10), Linker for activation of T-cells family member 1 (LAT), SH2 Domain-Containing Leukocyte Protein Of 76 kD (SLP76), T cell receptor associated transmembrane adaptor 1 (TRAT1), TNFR2, TNFRS14, TNFRS18, TNRFS25, zeta chain of T cell receptor associated protein kinase 70 (ZAP70), or a fragment or combination thereof.

56. The method of embodiment 54, wherein the costimulatory domain includes CD137 (4-1BB) or a fragment or combination thereof.

57. The method of any of embodiments 4-56, wherein the first fusion protein and/or the second fusion protein further include a transmembrane domain.

58. The method of embodiment 57, wherein the transmembrane domain of the first fusion protein and/or the second fusion protein is a CD4 transmembrane domain or a CD8α transmembrane domain.

59. The method of any of embodiments 4-58, wherein the first fusion protein and/or the second fusion protein further includes a spacer.

60. The method of any of embodiments 4-59, wherein:
(a) the first fusion protein includes: an FRB multimerization domain or variant thereof; a CD8α transmembrane domain or a CD4 transmembrane domain; a CD137 co-stimulatory domain; and/or a CD3ζ primary signaling domain; and
(b) the second fusion protein includes: a CD33 VHH that has an amino acid sequence as set forth in any one of SEQ ID NOs: 2-21; an FKBP multimerization domain or variant thereof; and a CD4 transmembrane domain or a CD8α transmembrane domain.

61. The method of any of embodiments 4-60, wherein the first fusion protein includes a signal peptide, a CD8α transmembrane domain; a CD137 co-stimulatory domain; and a CD3ζ primary signaling domain.

62. The method of any of embodiments 4-61, wherein the second fusion protein includes a signal peptide and a CD4 transmembrane domain.

63. The method of any of embodiments 4-62, wherein the first fusion protein and/or the second fusion protein has the sequence as set forth in any one of SEQ ID NOs: 32, 33, 34, 35, 36, 37, 38, 39, 40, or 41.

64. The method of any of embodiments 4-63, wherein the first fusion protein and/or the second fusion protein has at least 90% sequence identity to the sequence as set forth in any one of SEQ ID NOs: 32, 33, 34, 35, 36, 37, 38, 39, 40, or 41.

65. The method of any of embodiments 4-64, wherein the first fusion protein and/or the second fusion protein has the sequence as set forth in SEQ ID NO: 40.

66. The method of any of embodiments 4-65, wherein the first fusion protein and/or the second fusion protein has at least 90% sequence identity to the sequence as set forth in SEQ ID NO: 40.

67. The method of any of embodiments 4-66, wherein the first fusion protein and/or the second fusion protein has at least 95% sequence identity to the sequence as set forth in SEQ ID NO: 40.

68. The method of any of embodiments 4-67, wherein the first fusion protein and/or the second fusion protein has at least 98% sequence identity to the sequence as set forth in SEQ ID NO: 40.

69. The method of any of embodiments 4-68, wherein the rapamycin or the analog thereof includes rapamycin, AP1903, AP20187, AP21967, everolimus, novolimus, pimecrolimus, ridaforolimus, tacrolimus, temsirolimus, umirolimus, or zotarolimus.

70. The method of any of embodiments 4-69, wherein at least a subset of the cells expressing the DARIC in the identified subject are hematopoietic cells.

71. The method of any of embodiments 4-70, wherein at least a subset of the cells expressing the DARIC in the identified subject are T cells.

72. The method of any of embodiments 4-71, wherein at least a subset of the cells expressing the DARIC in the identified subject are αβ T cell or γδ T cell.

73. The method any of embodiments 4-72, wherein at least a subset of the cells expressing the DARIC in the identified subject are CD3+, CD4+, or CD8+ cells.

74. The method of any of embodiments 4-73, wherein at least a subset of the cells expressing the DARIC in the identified subject are immune effector cells, wherein at least a subset of the cells expressing the DARIC in the identified subject.

75. The method of any of embodiments 4-74, wherein at least a subset of the cells expressing the DARIC in the identified subject are cytotoxic T lymphocytes (CTLs), tumor infiltrating lymphocytes (TILs), or helper T cells.

76. The method of any of embodiments 4-75, wherein at least a subset of the cells expressing the DARIC in the identified subject are natural killer (NK) cells or natural killer T (NKT) cells.

77. The method of any of embodiments 4-76, wherein the cells were isolated from peripheral blood mononuclear cells, bone marrow, lymph nodes tissue, cord blood, thymus issue, tissue from a site of infection, ascites, pleural effusion, spleen tissue, or tumors.

78. The method of embodiment 77, wherein the cells were ex vivo manufactured to express the DARIC.

79. The method of embodiment 78, wherein the cells were administered to the subject at a dose of cells per weight of subject of from 1×10⁵ cells/kg to 2000×10⁶ cells/kg.

80. The method of embodiment 78, wherein the cells were administered to the subject at a dose of cells per weight of subject of from 1×10⁶ cells/kg to 1000×10⁶ cells/kg.

81. The method of embodiment 78, wherein the cells were administered to the subject at a dose of cells per weight of subject of from 1×10⁶ cells/kg to 100×10⁶ cells/kg.

82. The method of embodiment 78, wherein the cells were administered to the subject at a dose of cells per weight of subject of from 5×10⁶ cells/kg to 500×10⁶ cells/kg.

83. The method of embodiment 78, wherein the cells were administered to the subject at a dose of cells per weight of subject of from 10×10⁶ cells/kg to 1000×10⁶ cells/kg.

84. The method of embodiment 78, wherein the cells were administered to the subject at a dose of cells per weight of subject of from 1×10⁶ cells/kg to 2×10⁶ cells/kg.

85. The method of embodiment 78, wherein the cells were administered to the subject at a dose of cells per weight of subject of from 3×10⁶ cells/kg to 5×10⁶ cells/kg.

86. The method of embodiment 78, wherein the cells were administered to the subject at a dose of cells per weight of subject of from 7.5×10⁶ cells/kg to 15×10⁶ cells/kg.

87. The method of embodiment 78, wherein the dose of cells per weight of subject is 10×10⁶ cells/kg.

88. The method of any of embodiments 4-87, wherein the cells were modified in the subject in vivo to express the DARIC.

89. The method of any of embodiments 4-88, wherein the subject is a pediatric patient.

90. The method of any of embodiments 4-88, wherein the subject is no more than 28 years old.

91. The method of any of embodiments 4-88, wherein the subject is no more than 18 years old.

92. The method of any of embodiments 4-88, wherein the subject is from 18 to 28 years old.

93. The method of any of embodiments 4-88, wherein the subject is an adult patient.

94. The method of any of embodiments 4-88, wherein the subject is at least 18 years old.

95. The method of any of embodiments 4-94, wherein the subject has or is diagnosed with a cancer, infectious disease, autoimmune disease, inflammatory disease, an immunodeficiency, or a condition associated therewith.

96. The method of any of embodiments 4-95, wherein the subject has or is diagnosed with a solid cancer.

97. The method of embodiment 96, wherein the solid cancer includes lung cancer, squamous cell carcinoma, colorectal cancer, pancreatic cancer, breast cancer, thyroid cancer, bladder cancer, cervical cancer, esophageal cancer, ovarian cancer, gastric cancer, endometrial cancer, or brain cancer.

98. The method of embodiment 97, wherein the lung cancer is a non-small cell lung carcinoma.

99. The method of embodiment 97, wherein the squamous cell carcinoma is head and neck squamous cell carcinoma.

100. The method of embodiment 97, wherein the brain cancer includes gliomas, glioblastomas, or oligodendrogliomas.

101. The method of any of embodiments 4-100, wherein the subject has or is diagnosed with a hematological malignancy.

102. The method of embodiment 101, wherein the hematological malignancy is a leukemia, lymphoma, or multiple myeloma.

103. The method of any of embodiments 4-102, wherein the haematological malignancy is acute myelogenous leukemia (AML).

104. The method of any of embodiments 4-103, wherein the subject is lymphodepleted prior to administering the course.

105. The method of embodiment 104, wherein the lymphodepletion includes administering a dose of Fludarabine and a dose of Cyclophosphamide.

106. The method of embodiment 105, wherein the Fludarabine is administered at a dose of 30 mg/m² IV once daily for 4 days, and the Cyclophosphamide is administered at a dose of 500 mg/m² IV once daily for 2 days.

107. The method of any of embodiments 104-106, wherein the Cyclophosphamide is administered on days 3 and 4 of Fludarabine administration.

108. The method of any of embodiments 104-107, wherein the lymphodepletion begins 7 days prior to administering the course.

### Exemplary Embodiments—Set 2

1. A method for treating a subject including:
a) administering a dose of cells expressing a dimerizing agent regulated immunomodulatory complex (DARIC), wherein the DARIC includes:
   a first fusion protein including a multimerization domain, and
   a second fusion protein including a multimerization domain; and
b) administering a course of rapamycin or an analog thereof that binds and is disposed between the multimerization domain of the first fusion protein and the multimerization domain of the second fusion protein.

2. A method for treating a subject including:
a) editing cells of the subject to express a dimerizing agent regulated immunomodulatory complex (DARIC), wherein the DARIC comprises:
   a first fusion protein including a multimerization, and
   a second fusion protein including a multimerization domain; and
b) administering a course of rapamycin or an analog thereof that binds and is disposed between the multimerization domain of the first fusion protein and the multimerization domain of the second fusion protein.

3. A method for priming a dimerizing agent regulated immunomodulatory complex (DARIC) for signaling in a subject comprising:
a) administering a dose of cells expressing a DARIC, wherein the DARIC includes:
   a first fusion protein including a multimerization domain, and
   a second fusion protein including a multimerization domain; and
b) administering a course of rapamycin or an analog thereof the binds and is disposed between the multimerization domain of the first fusion protein and the multimerization domain of the second fusion protein.

4. A method for priming a dimerizing agent regulated immunomodulatory complex (DARIC) for signaling in a subject including:
a) genetically modifying cells of the subject to express DARIC, wherein the DARIC includes:
   a first fusion protein including a multimerization domain, and
   a second fusion protein including a multimerization domain; and
b) administering a course of rapamycin or an analog thereof that binds and is disposed between the multimerization domain of the first fusion protein and the multimerization domain of the second fusion protein.

5. The method of any of embodiments 1-4, wherein the second fusion protein further includes a binding domain.

6. The method of any of embodiments 1-4, wherein the first fusion protein further includes a binding domain.

7. The method of embodiment 5 or 6, wherein the binding domain binds a cancer antigen.

8. The method of any of embodiments 5-7, wherein the binding domain is a variable heavy chain (VHH) or a single chain variable fragment (scFv).

9. The method of any of embodiments 5-7, wherein the binding domain is a receptor.

10. The method of embodiment 9, wherein the receptor is the extracellular portion of a receptor.

11. The method of any of embodiments 1-10, wherein the second fusion protein further includes a spacer.

12. The method of any of embodiments 1-11, first fusion protein further includes an intracellular component including intracellular signaling domain.

13. The method of any of embodiments 1-12, wherein the multimerization domain of the first fusion protein includes an FKBP-rapamycin binding (FRB) multimerization domain or variant thereof, and the multimerization domain of the second fusion protein includes an FK506 binding protein (FKBP) multimerization domain or variant thereof.

14. The method of any of embodiments 1-12, wherein the multimerization domain of the first fusion protein includes an FK506 binding protein (FKBP) multimerization domain or a variant thereof, and the multimerization domain of the second fusion protein includes an FKBP-rapamycin binding (FRB) multimerization domain or a variant thereof.

15. The method of embodiment 13 or 14, wherein the FKBP multimerization domain or the variant thereof is FKBP12.

16. The method of embodiment 13 or 14, wherein the FRB multimerization domain or the variant thereof is FRB T2098L.

17. The method of any of embodiments 13-16, wherein the FRB multimerization domain and the FKBP multimerization domain localize extracellularly when the first fusion protein and the second fusion protein are expressed.

18. The method of any of embodiments 1-17, wherein the administering results in a target trough blood level of rapamycin or the analog thereof ranging from 0.5 ng/mL to 5 ng/mL.

19. The method of any of embodiments 1-17, wherein the administering results in a target trough blood level of rapamycin or the analog thereof ranging from 1 ng/mL to 5 ng/mL.

20. The method of any of embodiments 1-17, wherein the administering results in a target trough blood level of rapamycin or the analog thereof ranging from 1 ng/mL to 4 ng/mL.

21. The method of any of embodiments 1-17, wherein the administering results in a target trough blood level of rapamycin or the analog thereof ranging from 1 ng/mL to 3 ng/mL.

22. The method of any of embodiments 1-17, wherein the administering results in a target trough blood level of rapamycin or the analog thereof ranging from 1.5 ng/mL to 3 ng/mL.

23. The method of any of embodiments 1-22, wherein the administering results in a target trough blood level of rapamycin or the analog thereof of 0.5 ng/mL, 1 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, 2.0 ng/mL, 2.1 ng/mL, 2.2 ng/mL, 2.3 ng/mL, 2.4 ng/mL, 2.5 ng/mL, 2.6 ng/mL, 2.7 ng/mL, 2.8 ng/mL, 2.9 ng/mL, or 3.0 ng/mL

24. The method of any of embodiments 1-22, wherein the administering results in a target trough blood level of rapamycin or the analog thereof of 2 ng/mL.

25. The method of any of embodiments 1-24, wherein the subject has a body surface area greater than 1.5 m².

26. The method of embodiment 25, wherein a dose of rapamycin or the analog thereof during the course is from 0.75 mg to 4 mg.

27. The method of embodiment 25, wherein each dose or a majority of the doses of rapamycin or the analog thereof during the course are from 0.5 mg to 4 mg.

28. The method of embodiment 25, wherein a dose of rapamycin or the analog thereof during the course is from 0.75 mg to 3.5 mg.

29. The method of embodiment 25, wherein each dose or a majority of the doses of rapamycin or the analog thereof during the course are from 0.75 mg to 3.5 mg.

30. The method of embodiment 25, wherein a dose of rapamycin or the analog thereof during the course is from 0.75 mg to 1.5 mg.

31. The method of embodiment 25, wherein each dose or a majority of the doses of rapamycin or the analog thereof during the course are from 0.75 mg to 1.5 mg.

32. The method of embodiment 25, wherein a dose of rapamycin or the analog thereof during the course is from 0.75 mg to 2.0 mg.

33. The method of embodiment 25, wherein each dose or a majority of the doses of rapamycin or the analog thereof during the course are from 0.75 mg to 2.0 mg.

34. The method of embodiment 25, wherein a dose of rapamycin or the analog thereof during the course is from 0.75 mg to 2.5 mg.

35. The method of embodiment 25, wherein each dose or a majority of the doses of rapamycin or the analog thereof during the course are from 0.75 mg to 2.5 mg.

36. The method of embodiment 25, wherein a dose of rapamycin or the analog thereof during the course is 0.5 mg.

37. The method of embodiment 25, wherein each dose or a majority of the doses of rapamycin or the analog thereof during the course are 0.5 mg.

38. The method of embodiment 25, wherein a dose of rapamycin or the analog thereof during the course is 0.75 mg.

39. The method of embodiment 25, wherein each dose or a majority of the doses of rapamycin or the analog thereof during the course are 0.75 mg.

40. The method of embodiment 25, wherein each dose or a majority of the doses of rapamycin or the analog thereof during the course are 1.0 mg.

41. The method of embodiment 25, wherein each dose or a majority of the doses of rapamycin or the analog thereof during the course are 1.0 mg.

42. The method of embodiment 25, wherein a dose of rapamycin or the analog thereof during the course is 1.5 mg.

43. The method of embodiment 25, wherein each dose or a majority of the doses of rapamycin or the analog thereof during the course are 1.5 mg.

44. The method of embodiment 25, wherein a dose of rapamycin or the analog thereof during the course is 2.0 mg.

45. The method of embodiment 25, wherein each dose or a majority of the doses of rapamycin or the analog thereof during the course are 2.0 mg.

46. The method of embodiment 25, wherein a dose of rapamycin or the analog thereof during the course is 2.5 mg.

47. The method of embodiment 25, wherein each dose or a majority of the doses of rapamycin or the analog thereof during the course are 2.5 mg.

48. The method of embodiment 25, wherein a dose of rapamycin or the analog thereof during the course is 3.0 mg.

49. The method of embodiment 25, wherein each dose or a majority of the doses of rapamycin or the analog thereof during the course are 3.0 mg.

50. The method of embodiment 25, wherein a dose of rapamycin or the analog thereof during the course is 3.5 mg.

51. The method of embodiment 25, wherein each dose or a majority of the doses of rapamycin or the analog thereof during the course are 3.5 mg.

52. The method of embodiment 25, wherein a dose of rapamycin or the analog thereof during the course is 0.8 mg.

53. The method of embodiment 25, wherein each dose or a majority of the doses of rapamycin or the analog thereof during the course is 0.8 mg/m.

54. The method of embodiment 25, wherein a dose of rapamycin or the analog thereof during the course is 0.7 mg.

55. The method of embodiment 25, wherein each dose or a majority of the doses of rapamycin or the analog thereof during the course is 0.7 mg.

56. The method of embodiment 25, wherein a dose of rapamycin or the analog thereof during the course is 0.6 mg.

57. The method of embodiment 25, wherein each dose or a majority of the doses of rapamycin or the analog thereof is 0.6 mg.

58. The method of embodiment 25, wherein a dose of rapamycin or the analog thereof during the course is higher than 0.75 mg.

59. The method of embodiment 25, wherein each dose or a majority of the doses of rapamycin or the analog thereof during the course higher than 0.75 mg.

60. The method of embodiment 25, wherein a dose of rapamycin or the analog thereof during the course is higher than 1.0 mg.

61. The method of embodiment 25, wherein each dose or a majority of the doses of rapamycin or the analog thereof during the course is higher than 1.0 mg.

62. The method of embodiment 25, wherein a dose of rapamycin or the analog thereof during the course is higher than 1.5 mg.

63. The method of embodiment 25, wherein each dose or a majority of the doses of rapamycin or the analog thereof during the course is higher than 1.5 mg.

64. The method of embodiment 25, wherein a dose of rapamycin or the analog thereof during the course is higher than 2.0 mg.

65. The method of embodiment 25, wherein each dose or a majority of the doses of rapamycin or the analog thereof during the course is higher than 2.0 mg.

66. The method of embodiment 25, wherein a dose of rapamycin or the analog thereof during the course is higher than 2.5 mg.

67. The method of embodiment 25, wherein each dose or a majority of the doses of rapamycin or the analog thereof during the course is higher than 2.5 mg.

68. The method of embodiment 25, wherein a dose of rapamycin or the analog thereof during the course is higher than 3.0 mg.

69. The method of embodiment 25, wherein each dose or a majority of the doses of rapamycin or the analog thereof during the course is higher than 3.0 mg.

70. The method of embodiment 25, wherein a dose of rapamycin or the analog thereof during the course is higher than 3.5 mg.

71. The method of embodiment 25, wherein each dose or a majority of the doses of rapamycin or the analog thereof during the course is higher than 3.5 mg.

72. The method of embodiment 25, wherein a dose of rapamycin or the analog thereof during the course is 3.0-4.0 mg.

73. The method of embodiment 25, wherein each dose or a majority of the doses of rapamycin or the analog thereof during the course is 3.0-4.0 mg.

74. The method of embodiment 25, wherein a dose of rapamycin or the analog thereof during the course is 3.5-4.5 mg.

75. The method of embodiment 25, wherein each dose or a majority of the doses of rapamycin or the analog thereof during the course is 3.5-4.5 mg.

76. The method of any of embodiments 1-24, wherein the subject has a body surface area less than or equal to 1.5 m².

77. The method of embodiment 76, wherein a dose of rapamycin or the analog thereof during the course is from 0.2 mg/m² to 0.75 mg/m².

78. The method of embodiment 76, wherein each dose or a majority of the doses of rapamycin or the analog thereof during the course are from 0.2 mg/m² to 0.75 mg/m².

79. The method of embodiment 76, wherein a dose of rapamycin or the analog thereof during the course is from 0.3 mg/m² to 0.7 mg/m².

80. The method of embodiment 76, wherein each dose or a majority of the doses of rapamycin or the analog thereof during the course are from 0.3 mg/m² to 0.7 mg/m².

81. The method of embodiment 76, wherein a dose of rapamycin or the analog thereof during the course is from 0.4 mg/m² to 0.6 mg/m².

82. The method of embodiment 76, wherein each dose or a majority of the doses of rapamycin or the analog thereof during the course are from 0.4 mg/m² to 0.6 mg/m².

83. The method of embodiment 76, wherein a dose of rapamycin or the analog thereof during the course is from 0.1 mg/m² to 0.5 mg/m².

84. The method of embodiment 76, wherein each dose or a majority of the doses of rapamycin or the analog thereof during the course are from 0.1 mg/m² to 0.5 mg/m².

85. The method of embodiment 76, wherein a dose of rapamycin or the analog thereof during the course is from 0.5 mg/m² to 0.7 mg/m².

86. The method of embodiment 76, wherein each dose or a majority of the doses of rapamycin or the analog thereof during the course are from 0.5 mg/m² to 0.7 mg/m².

87. The method of embodiment 76, wherein a dose of rapamycin or the analog thereof during the course is 0.1 mg/m².

88. The method of embodiment 76, wherein each dose or a majority of the doses of rapamycin or the analog thereof during the course are 0.1 mg/m².

89. The method of embodiment 76, wherein a dose of rapamycin or the analog thereof during the course is 0.2 mg/m².

90. The method of embodiment 76, wherein each dose or a majority of the doses of rapamycin or the analog thereof during the course are 0.2 mg/m².

91. The method of embodiment 76, wherein a dose of rapamycin or the analog thereof during the course is 0.3 mg/m².

92. The method of embodiment 76, wherein each dose or a majority of the doses of rapamycin or the analog thereof during the course are 0.3 mg/m².

93. The method of embodiment 76, wherein a dose of rapamycin or the analog thereof during the course is 0.4 mg/m².

94. The method of embodiment 76, wherein each dose or a majority of the doses of rapamycin or the analog thereof during the course are 0.4 mg/m².

95. The method of embodiment 76, wherein a dose of rapamycin or the analog thereof during the course is 0.5 mg/m².

96. The method of embodiment 76, wherein each dose or a majority of the doses of rapamycin or the analog thereof during the course are 0.5 mg/m².

97. The method of embodiment 76, wherein a dose of rapamycin or the analog thereof during the course is 0.6 mg/m².

98. The method of embodiment 76, wherein each dose or a majority of the doses of rapamycin or the analog thereof during the course are 0.6 mg/m².

99. The method of embodiment 76, wherein a dose of rapamycin or the analog thereof during the course is 0.7 mg/m².

100. The method of embodiment 76, wherein each dose or a majority of the doses of rapamycin or the analog thereof during the course are 0.7 mg/m².

101. The method of embodiment 76, wherein a dose of rapamycin or the analog thereof during the course is 0.75 mg/m².

102. The method of any of embodiments 26-101, wherein the dose of rapamycin or the analog thereof is administered once a day.

103. The method of any of embodiments 26-101, wherein the dose of rapamycin or the analog thereof is administered twice a day.

104. The method of any of embodiments 1-102, wherein the course is daily administration for 10 to 28 days.

105. The method of any of embodiments 1-102, wherein the course is daily administration for 11 to 27 days.

106. The method of any of embodiments 1-102, wherein the course is daily administration for 12 to 26 days.

107. The method of any of embodiments 1-102, wherein the course is daily administration for 13 to 25 days.

108. The method of any of embodiments 1-102, wherein the course is daily administration for 14 to 24 days.

109. The method of any of embodiments 1-102, wherein the course is daily administration for 15 to 23 days.

110. The method of any of embodiments 1-102, wherein the course is daily administration for 16 to 22 days.

111. The method of any of embodiments 1-102, wherein the course is daily administration for 17 to 21 days.

112. The method of any of embodiments 1-102, wherein the course is daily administration for 18 to 20 days.

113. The method of any of embodiments 1-102, wherein the course is daily administration for 10, 11, 12, 13, 14, 15, 16, 17, 18, 19, 20, 21, 22, 23, 24, 25, 26, 27, or 28 days.

114. The method of any of embodiments 1-113, wherein the course is daily administration for 19 days.

115. The method of any of embodiments, 1-114, wherein the course begins at least 16 hours after the subject first has in vivo cells expressing the DARIC.

116. The method of any of embodiments 1-114, wherein the course does not begin for at least 24 hours after the subject first has in vivo cells expressing the DARIC (due to, for example, infusion of cells expressing the DARIC).

117. The method of any of embodiments 1-114, wherein the course does not begin for at least 36 hours after the subject first has in vivo cells expressing the DARIC.

118. The method of any of embodiments 1-114, wherein the course does not begin for at least 48 hours after the subject first has in vivo cells expressing the DARIC.

119. The method of any of embodiments 1-114, wherein the course does not begin for at least 60 hours after the subject first has in vivo cells expressing the DARIC.

120. The method of any of embodiments 1-114, wherein the course does not begin for at least 72 hours after the subject first has in vivo cells expressing the DARIC.

121. The method of any of embodiments 1-114, wherein the course does not begin for at least 84 hours after the subject first has in vivo cells expressing the DARIC.

122. The method of any of embodiments 1-114, wherein the course begins 1 to 4 days after the subject first has in vivo cells expressing the DARIC.

123. The method of any of embodiments 1-114, wherein the course begins 1 to 3 days after the subject first has in vivo cells expressing the DARIC.

124. The method of any of embodiments 1-114, wherein the course begins 1 to 2 days after the subject first has in vivo cells expressing the DARIC.

125. The method of any of embodiments 1-114, wherein the course begins 2 to 4 days after the subject first has in vivo cells expressing the DARIC.

126. The method of any of embodiments 1-114, wherein the course begins 3 to 4 days after the subject first has in vivo cells expressing the DARIC.

127. The method of any of embodiments 1-114, wherein the course begins 2 to 3 days after the subject first has in vivo cells expressing the DARIC.

128. The method of any of embodiments 1-127, wherein the method includes a rest period of no rapamycin or analog thereof administration after the course.

129. The method of embodiment 128, wherein the rest period is 10-45 days.

130. The method of embodiment 128, wherein the rest period is 14-25 days.

131. The method of embodiment 128, wherein the rest period is 14, 15, 16, 17, 18, 19, 20, 21, 22, 23, 24, or 25 days.

132. The method of embodiment 128, wherein the rest period is at least 14 days.

133. The method of any of any of embodiments 128-132, wherein a subsequent course of rapamycin or the analog thereof is administered to the subject after the rest period.

134. The method of embodiment 133, where the subsequent course matches a course of any of the preceding embodiments.

135. The method of embodiment 134, followed by a second rest period.

136. The method of embodiment 135, wherein the rest period matches a rest period of any of embodiments 128-135.

137. The method of embodiment 135 or 136, where a second subsequent course of rapamycin or the analog thereof is administered to the subject after the second rest period.

138. The method of embodiment 137, wherein the second subsequent course matches a course of any of the preceding embodiments.

139. The method of any of embodiments 1-138, wherein the rapamycin or the analog thereof includes rapamycin, AP1903, AP20187, AP21967, everolimus, novolimus, pimecrolimus, ridaforolimus, tacrolimus, temsirolimus, umirolimus, zotarolimus, or BPC015.

140. The method of any of embodiments 1-4 or 11-139, wherein the first and/or second fusion protein include a binding domain.

141. The method any of embodiments 5, 6, 7, or 140 wherein the binding domain includes a binding domain of a CD33 antibody.

142. The method of embodiment 141, wherein the binding domain of the CD33 antibody is a VHH.

143. The method of embodiment 142, wherein the VHH has the sequence as set forth in any one of SEQ ID NOs: 2, 3, 4, 5, 6, 7, 8, 9, 10, 11, 12, 13, 14, 15, 16, 17, 18, 19, 20, or 21.

144. The method of embodiment 142, wherein the VHH has at least 90% sequence identity to the sequence as set forth in any one of SEQ ID NOs: 2, 3, 4, 5, 6, 7, 8, 9, 10, 11, 12, 13, 14, 15, 16, 17, 18, 19, 20, or 21 and specifically binds CD33.

145. The method of any of embodiments 1-142, wherein the first fusion protein and/or the second fusion protein include a binding domain that binds CLL1.

146. The method of embodiment 145, wherein the binding domain that binds CLL1 has the sequence as set forth in any one of SEQ ID NOs: 43, 44, 45, 46, 47, 48, 49, 50, 51, 52, 53 or 54.

147. The method of embodiment 145, wherein the binding domain that binds CLL1 has at least 90% sequence identity to the sequence as set forth in any one of SEQ ID NOs: 43, 44, 45, 46, 47, 48, 49, 50, 51, 52, 53 or 54 and specifically binds CLL1.

148. The method of any of embodiments 1-147, wherein the first fusion protein includes a CD8a transmembrane domain and the second fusion protein includes a CD4 transmembrane domain.

149. The method of any of embodiments 1-148, wherein the first fusion protein includes an internal component.

150. The method of embodiment 149, wherein the intracellular component includes a primary signaling domain.

151. The method of embodiment 150, wherein the primary signaling domain is CD3ζ or a fragment thereof.

152. The method of any of embodiments 149-151, wherein the intracellular component includes a costimulatory domain.

153. The method of embodiment 152, wherein the costimulatory domain includes Toll-like receptor 1 (TLR1), TLR2, TLR3, TLR4, TLR5, TLR6, TLR7, TLR8, TLR9, TLR10, caspase recruitment domain family member 11 (CARD11), CD2, CD7, CD27, CD28, CD30, CD40, CD54 (ICAM), CD83, CD94, CD134 (OX40), CD137 (4-1BB), CD278 (ICOS), DNAX-Activation Protein 10 (DAP10), Linker for activation of T-cells family member 1 (LAT), SH2 Domain-Containing Leukocyte Protein Of 76 kD (SLP76), T cell receptor associated transmembrane adaptor 1 (TRAT1), TNFR2, TNFRS14, TNFRS18, TNRFS25, zeta chain of T cell receptor associated protein kinase 70 (ZAP70), or a fragment or combination thereof.

154. The method of embodiment 152, wherein the costimulatory domain OX40 or TNFR2 or a fragment or combination thereof.

155. The method of any of embodiments 1-154 wherein:
(a) the first fusion protein includes: an FRB multimerization domain or variant thereof; a CD8α transmembrane domain or a CD4 transmembrane domain; a CD137 co-stimulatory domain; and/or a CD3ζ primary signaling domain; and
(b) the second fusion protein includes: a CD33 VHH that has an amino acid sequence as set forth in any one of SEQ ID NOs: 2-21; an FKBP multimerization domain polypeptide or variant thereof; and a CD4 transmembrane domain or a CD8α transmembrane domain.

156. The method of any of embodiments 1-155, wherein the first or second fusion protein has the sequence as set forth in in any one of SEQ ID NOs: 32, 33, 34, 35, 36, 37, 38, 39, 40, or 41.

157. The method of any of embodiments 1-155, wherein the first fusion protein and/or the second fusion protein has at least 90% sequence identity to the sequence as set forth in any one of SEQ ID NOs: 32, 33, 34, 35, 36, 37, 38, 39, 40, or 41.

158. The method of any of embodiments 1-155, wherein the first fusion protein and/or the second fusion protein has the sequence as set forth in SEQ ID NO: 40.

159. The method of any of embodiments 1-155, wherein the first fusion protein and/or the second fusion protein has at least 90% sequence identity to the sequence as set forth in SEQ ID NO: 40.

160. The method of any of embodiments 1-159, wherein at least a subset of the cells are hematopoietic cells.

161. The method of any of embodiments 1-160, wherein at least a subset of the cells are T cells.

162. The method of any of embodiments 1-161, wherein at least a subset of the cells are αβ T cell or γδ T cell.

163. The method of any of embodiments 1-162, wherein at least a subset of the cells are CD3+, CD4+, or CD8+ cells.

164. The method of any of embodiments 1-163, wherein at least a subset of the cells are immune effector cells.

165. The method of any of embodiments 1-164, wherein at least a subset of the cells are cytotoxic T lymphocytes (CTLs), tumor infiltrating lymphocytes (TILs), or helper T cells.

166. The method of any of embodiments 1-165, wherein at least a subset of the cells are natural killer (NK) cells or natural killer T (NKT) cells.

167. The method of any of embodiments 1-166, wherein the cells were isolated from peripheral blood mononuclear cells, bone marrow, lymph nodes tissue, cord blood, thymus issue, tissue from a site of infection, ascites, pleural effusion, spleen tissue, or tumors.

168. The method of any of embodiments 1, 3, or 5-167, wherein the dose of cells per weight of subject is from 1×10⁵ cells/kg to 2000×10⁶ cells/kg.

169. The method of any of embodiments 1, 3, or 5-167, wherein the dose of cells per weight of subject is from 1×10⁶ cells/kg to 1000×10⁶ cells/kg.

170. The method of any of embodiments 1, 3, or 5-167, wherein the dose of cells per weight of subject is from 1×10⁶ cells/kg to 100×10⁶ cells/kg.

171. The method of any of embodiments 1, 3, or 5-167, wherein the dose of cells per weight of subject is from 5×10⁶ cells/kg to 500×10⁶ cells/kg.

172. The method of any of embodiments 1, 3, or 5-167, wherein the dose of cells per weight of subject is from 10×10⁶ cells/kg to 1000×10⁶ cells/kg.

173. The method of any of embodiments 1, 3, or 5-167, wherein the dose of cells per weight of subject is from 1×10⁶ cells/kg to 2×10⁶ cells/kg.

174. The method of any of embodiments 1, 3, or 5-167, wherein the dose of cells per weight of subject is from 3×10⁶ cells/kg to 5×10⁶ cells/kg.

175. The method of any of embodiments 1, 3, or 5-167, wherein the dose of cells per weight of subject is from 7.5×10⁶ cells/kg to 15×10⁶ cells/kg.

176. The method of any of embodiments 1, 3, or 5-167, wherein the dose of cells per weight of subject is 10×10⁶ cells/kg.

177. The method of any of embodiments 1-176, wherein the subject is a pediatric patient.

178. The method of any of embodiments 1-176, wherein the subject is no more than 28 years old.

179. The method of any of embodiments 1-176, wherein the subject is no more than 18 years old.

180. The method of any of embodiments 1-176, wherein the subject is from 18 to 28 years old.

181. The method of any of embodiments 1-176, wherein the subject has or is diagnosed with a cancer, infectious disease, autoimmune disease, inflammatory disease, an immunodeficiency, or a condition associated therewith.

182. The method of any of embodiments 1-181, wherein the subject has or is diagnosed with a solid cancer.

183. The embodiment 182, wherein the solid cancer includes lung cancer, squamous cell carcinoma, colorectal cancer, pancreatic cancer, breast cancer, thyroid cancer, bladder cancer, cervical cancer, esophageal cancer, ovarian cancer, gastric cancer, endometrial cancer, or brain cancer.

184. The method of embodiment 183, wherein the lung cancer is a non-small cell lung carcinoma.

185. The method of embodiment 183, wherein the squamous cell carcinoma is head and neck squamous cell carcinoma.

186. The method of embodiment 183, wherein the brain cancer includes gliomas, glioblastomas, or oligodendrogliomas.

187. The method of any of embodiments 1-186, wherein the subject has or is diagnosed with a hematological malignancy.

188. The method of embodiment 187, wherein the hematological malignancy is a leukemia, lymphoma, or multiple myeloma.

189. The method of embodiment 188, wherein the haematological malignancy is acute myelogenous leukemia (AML).

190. The method of any of embodiments 1-189, wherein the subject is lymphodepleted prior to administering the cells or genetically modifying the cells.

191. The method of embodiment 190, wherein the lymphodepletion includes administering a dose of Fludarabine and a dose of Cyclophosphamide.

192. The method of embodiment 191, wherein the Fludarabine is administered at a dose of 30 mg/m² IV once daily for 4 days, and the Cyclophosphamide is administered at a dose of 500 mg/m² IV once daily for 2 days.

193. The method of embodiment 190 or 191, wherein the Cyclophosphamide is administered on days 3 and 4 of Fludarabine administration.

194. The method of any of embodiments 191-193, wherein the lymphodepletion begins 5 days prior to administering the cells or genetically modifying the cells.

195. The method of any of embodiments 1-194, having an FRB multimerization domain and an FKBP multimerization domain (which can be “the” FRB multimerization domain and “the” FKBP multimerization domain of a preceding embodiment, but does not have to be), wherein the FRB multimerization domain and the FKBP multimerization domain localize intracellularly when the first fusion protein and the second fusion protein are expressed.

### (ix) EXPERIMENTAL EXAMPLES

### Example 1: DARIC33 Generation

Despite the successes of CAR T cell therapies, safety and efficacy often remain a challenge. Safety challenges include cytokine release syndrome, neurotoxicity and concern for aplasia due to expression of CD33 on normal hematopoietic tissue. Efficacy challenges include relapse due to antigen escape and T cell exhaustion. Next generation cell therapy designs can potentially address these concerns by providing a platform that can allow for controlled cell activation.

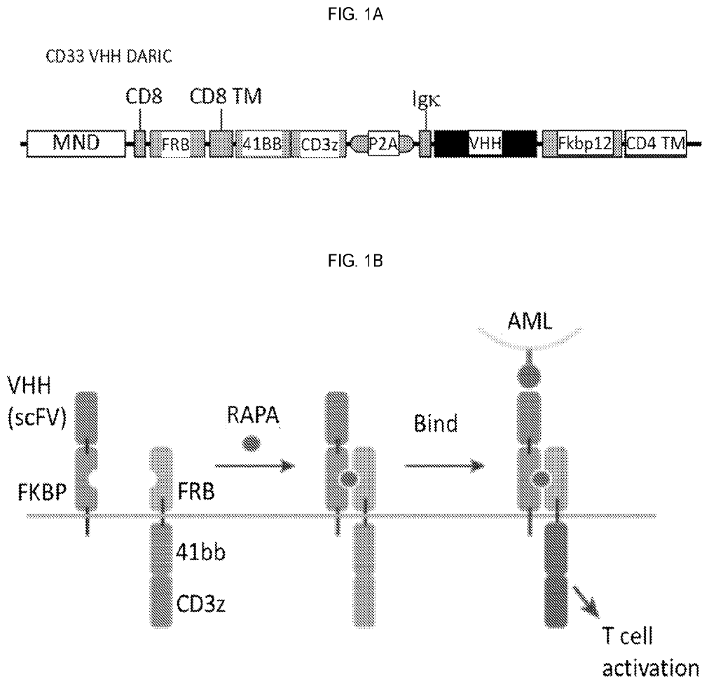

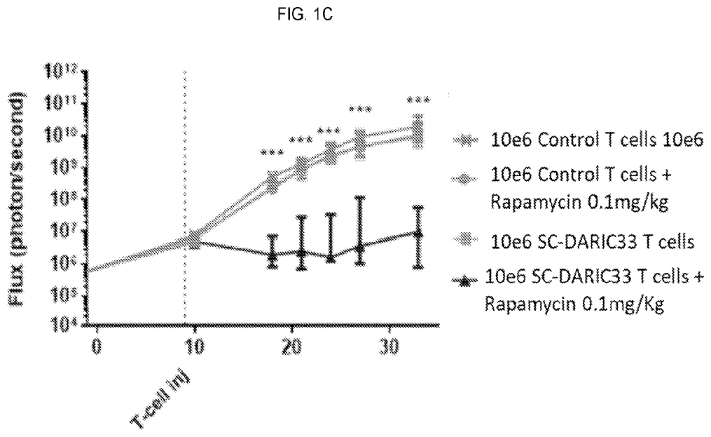

Here, anti-CD33 VHH DARIC binding and signaling components were designed, constructed, and verified. CD33 specific VHH DARIC lentiviral vectors were constructed including an MNDU3 promoter operably linked to a polynucleotide encoding: a DARIC signaling component (CD8α-signal peptide, an FRB variant (T82L), a CD8α transmembrane domain, an intracellular 4-1BB costimulatory domain, and a CD3 zeta signaling domain); a P2A sequence; and a DARIC binding component (an Igκ-signal peptide, a CD33 specific VHH binding domain (camelid or humanized), a G4S linker, an FKBP12 domain, and a CD4 derived transmembrane domain with a truncated intracellular signaling domain (FIG. 1A; SEQ ID NO: 40). See also, e.g., SEQ ID NOs: 32-41. See also WO 2015/017214 and WO 2020/227474. FIG. 1B depicts a SC-DARIC33 construct and rapamycin activation thereof.

To further demonstrate the efficacy of SC-DARIC33 to provide tumor control, two in vivo models were developed of adoptive engineered cell therapy of tumor cell line xenografts. Tumor cell lines were engineered to express a luciferase transgene to allow monitoring of tumor progression by bioluminescence.

In a first model, immunodeficient NSG mice engrafted with the AML cell line MV4-11 (CD33+) were treated with 10e6 SC-DARIC33 with or without rapamycin. 10e6 Control T cells not subjected to lentiviral transduction were included as a control. Rapid increases in tumor flux were seen in mice treated with control T cells with or without rapamycin as well as in mice treated with 10⁶ SC-DARIC33 T cells without rapamycin. Mice treated with SC-DARIC33 with rapamycin showed suppressed tumor growth over the 36-day study period. Among SC-DARIC33 treated mice who also received rapamycin, no statistically significant increase in tumor flux was seen following adoptive transfer of the SC-DARIC33 cells for the duration of the study (vs the maximal pre-treatment time point FIG. 1C).

### Example 2: CAR-T Dose Determination

To determine an appropriate starting dose for the SC-DARIC33 T Cell Product, it was sought to compare the efficacy of a previously developed CD19 CAR T cell product to SC-DARIC33.

To design a comparative model of CD19 and CD33-targeted T cell therapies, a Raji Non-Hodgkin lymphoma cell line was transduced with a vector directing expression of CD33 and eGFP:ff/luc to create Raji.CD33. The resulting cell line expresses CD19 and CD33 at similar antigen densities and following injection into NSG mice develops disseminated progressive disease which is usually fatal within 30 days if untreated. Matched clinical scale T cell products as well as a Mock T cell product from the same donor were generated next.

Immunocompromised mice were engrafted with 5×10⁵ Raji.CD33 cells and following confirmation of tumor growth were treated with 10e6 or 30e6 SC-DARIC33 T cell (with or without rapamycin), CD19 CAR T cells or mock T cells 7 days later. Tumor progression was monitored by bioluminescence for 33 days.

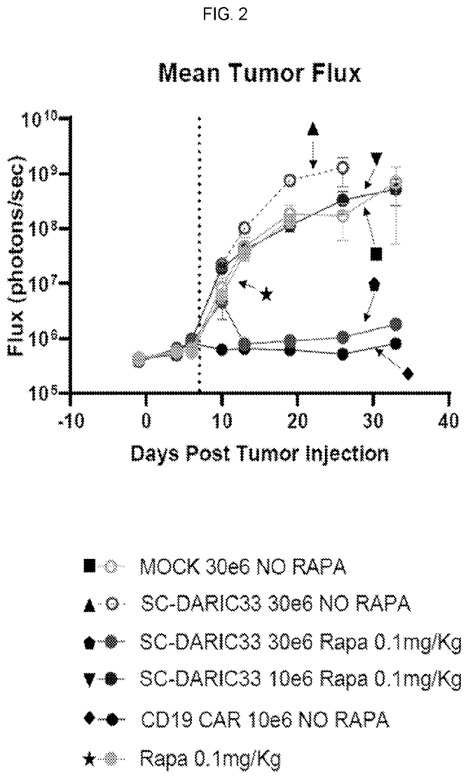

As shown in FIG. 2, there was a rapid increase in tumor-expressed bioluminescence among mice treated with either rapamycin alone, or control (mock transduced) T cells following tumor injection. Mice receiving SC-DARIC33 without rapamycin also showed increases in tumor flux to a similar rate and degree. Mice treated with 30e6 SC-DARIC33 T cells and rapamycin showed suppression of tumor growth to an extent similar to mice who received 10e6 CD19 CAR T cells. Mice treated with 10e6 SC-DARIC33 T cells and rapamycin did not show suppression of tumor growth, suggesting that this dose of SC-DARIC33 was less effective.

### Example 3: Rapamycin-Induced DARIC33 Activity Against Tumor Xenografts

To determine optimal clinical dosing of rapamycin for DARIC33 activation, it was necessary to determine the concentrations of rapamycin needed to activate DARIC33 in vivo.

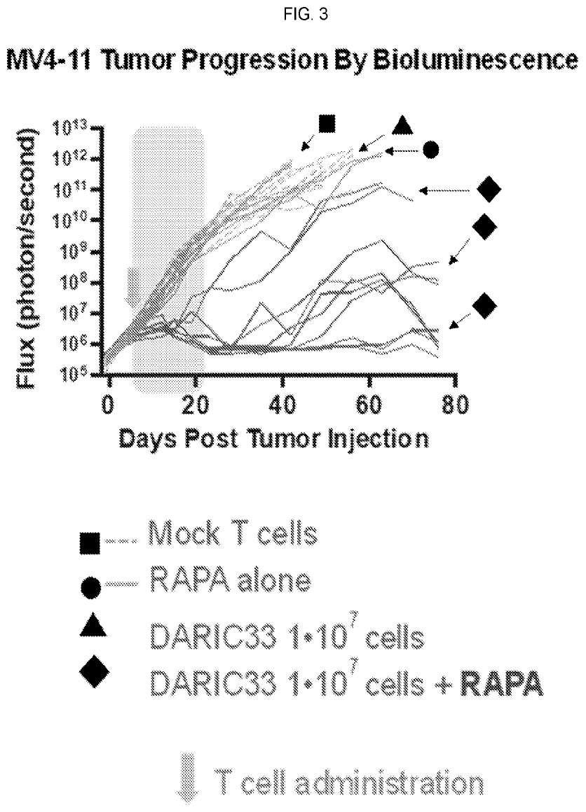

The AML cell line (MV-4-11) (modified to express luciferase for in vivo bioluminescence tracking) was adoptively transferred to NSG mice. Tumor engrafted mice were then treated with 10⁷ DARIC33 T cells or an equivalent number of untransduced T cells as a control. Following T cell injection, mice were treated with 0.01 mg/kg rapamycin via IP injection daily on days 1-20. Tumor growth was tracked by bioluminescence twice weekly. It was observed that 6 of 8 mice treated with DARIC33 T cells and rapamycin exhibited suppressed tumor growth, whereas all mice not treated with rapamycin, mice treated with rapamycin but not DARIC33 T cells, and mice treated with untransduced control T cells all exhibited rapid tumor growth (FIG. 3).

### Example 4: Assessing Reversibility of Rapamycin Induced Activation of DARIC33 T Cells

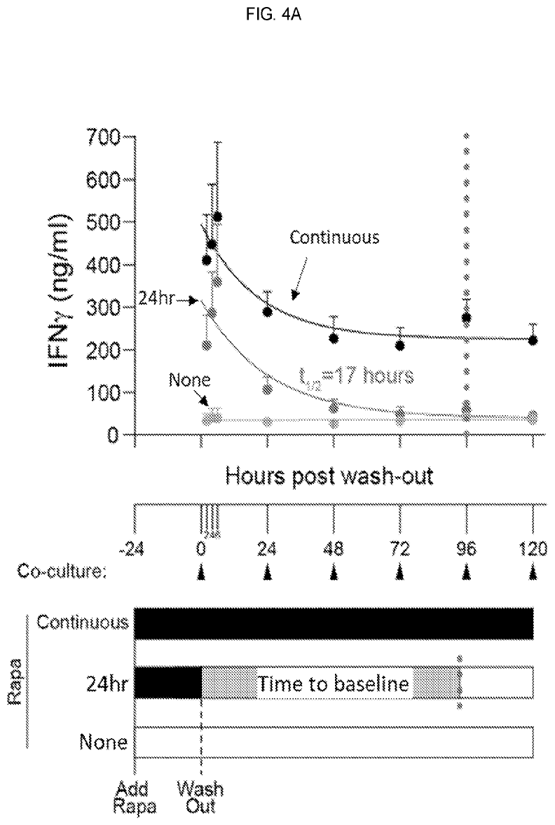

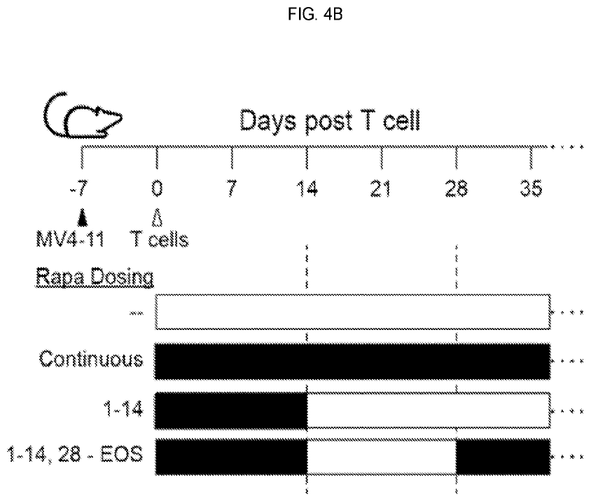

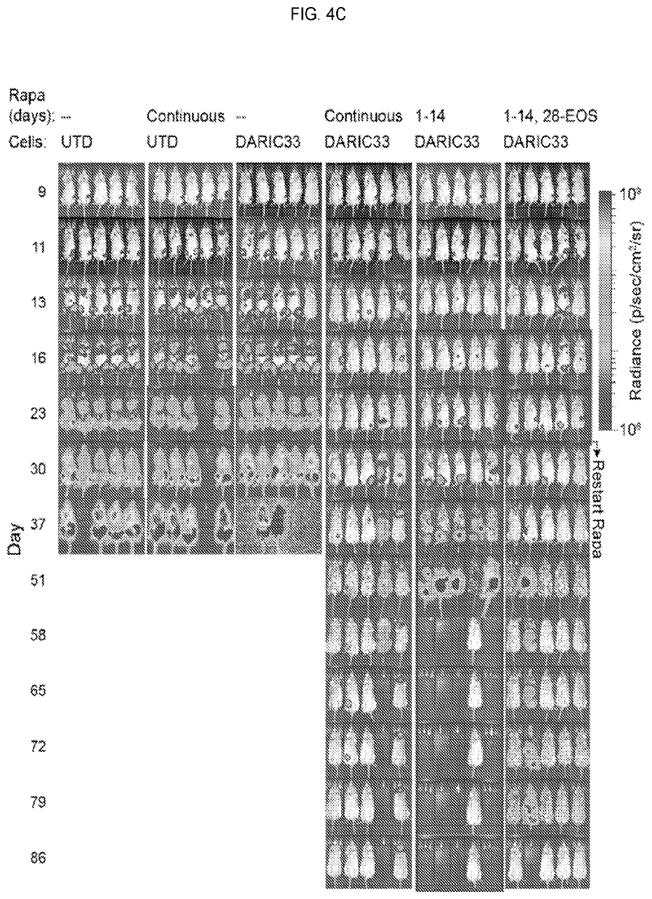

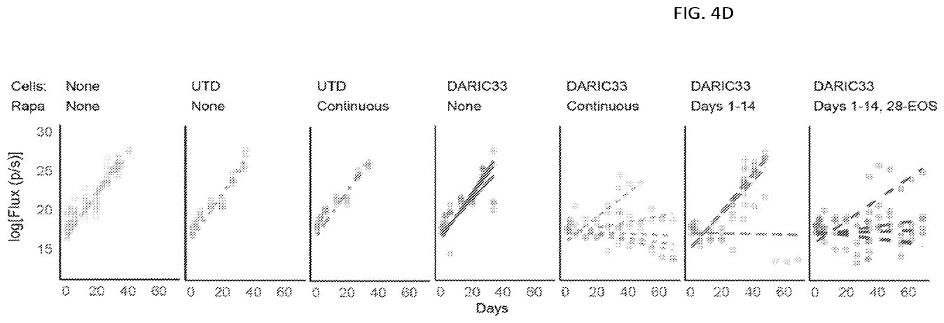

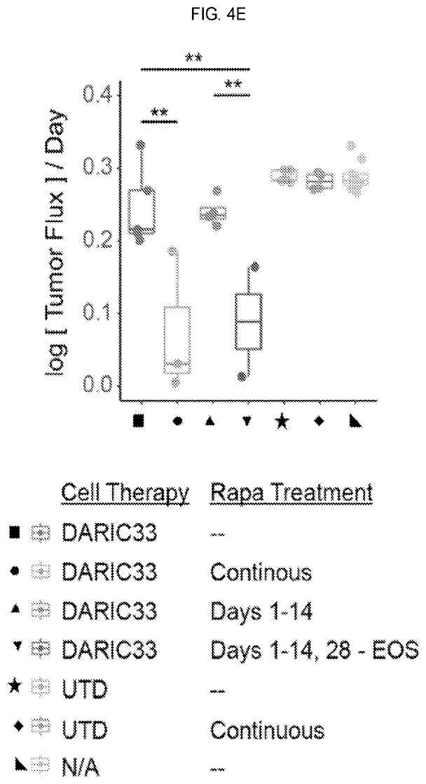

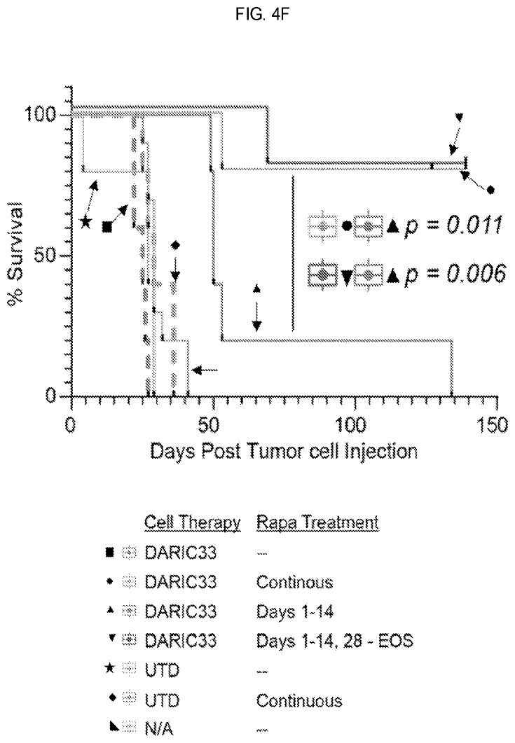

The capacity to temporarily pause DARIC33 T cell effector function in patients following cell dosing represents a control feature for toxicity mitigation and hematopoietic recovery. Moreover, therapeutic T cells that are intermittently rested may be less prone to functional exhaustion and capable of repopulating memory cell compartments. Therefore, to define kinetic effects of rapamycin removal, 50,000 DARIC33 T cells cultured with rapamycin for 24 hours were washed with rapamycin-free media prior to co-culture with 50,000 CD33+ MV4-11 AML target cells in rapamycin-free media. As a control, 50,000 DARIC33 T cells were continuously maintained in the presence of rapamycin and co-cultured at a 1:1 with the same MV-4-11 AML tumor line. Samples were taken after incubation at 37° C. at various time points (0 h, 2 h, 4 h, 6 h, 24 h, 48 h, 72 h, 96 h and 120 h) post co-culture initiation to determine activity. At early time points, pre-incubated SC-DARIC33 T cells showed high levels of activity measured by IFNγ release, followed by a progressive decline in activity, again measured by IFN^{γ} release, that returned to baseline within 96 hours and followed first-order kinetics characterized by a half-life of 17 hours (FIG. 4A). The t½ was determined by fitting a single-phase exponential decay.

To establish reversibility of SC-DARIC33 T cell activation in vivo, mice bearing AML xenografts derived from MV4-11 modified for BLI were treated with 107 SC-DARIC33+ T cells, or the equivalent number of UTD control cells. T cells were infused intravenously (IV) in NSG mice 7 days after engraftment of 1×10⁶ MV4-11.ff/luc leukemia cells. Following T cell infusion, mice were treated with 0.1 mg/kg rapamycin 3 times weekly for the indicated durations. Specifically, rapamycin was delivered following continuous (Days 1-150), interrupted (Days 1-14 and 28-150), or abbreviated (days 1-14) schedules (see FIG. 4B for schema). Whereas mice receiving UTD control cells (with or without rapamycin) exhibited tumor growth and tumor associated symptoms by day 50, mice treated with SC-DARIC33 cells and rapamycin exhibited delayed tumor progression (FIG. 4C), and prolonged symptom-free survival (FIG. 4F). Four of 5 mice receiving the abbreviated rapamycin schedule exhibited tumor relapses 3 weeks after rapamycin was discontinued. In contrast, when rapamycin was reinitiated, 4 of 5 mice controlled the tumor through the end of the observation period. Estimates of tumor growth kinetics, modeled using linear mixed effects, revealed similar growth rates among mice receiving inactive treatments and similar control of tumor growth among mice receiving continuous and intermittent rapamycin schedules (FIG. 4D and FIG. 4E). Suppression of AML growth by SC-DARIC33 cells correlated with an increase in survival (FIG. 4E). These data are consistent with a model wherein discontinuation of rapamycin pauses SC-DARIC33 antitumor activity, which may be restored by resuming rapamycin exposure.

### Example 5: In Vitro Modeling of DARIC33 T Cell Response to Rapamycin

To support rapamycin dose selection for first-in-human testing of SC-DARIC33, it was sought to define rapamycin concentrations required for SC-DARIC33 activation in situ. To begin, cytokine release following stimulation of DARIC33 cells over night with MV4-11 AML cells in media (X-VIVO) human whole blood, or mouse whole blood in the presence of increasing rapamycin concentrations was measured.

Specifically, quantification of the amount (concentration) of rapamycin that results in the half-maximal IFNγ cytokine release by DARIC33 T cells after co-culture with antigen positive (CD33+) target cells present in, or added to, human whole blood or mouse whole blood, was performed.

Healthy donor peripheral blood mononuclear cells (PBMC) were thawed, activated with anti-CD3 and anti-CD28 antibodies and transduced with DARIC33 A07-LV and control vector and expanded in G-Rex24 multi-well culture plates. On day 10 after initiation of T cell production, T cells were harvested, washed, and 50,000 DARIC T cells added to plates at a 1:1 effector to target ratio with the acute myeloid leukemia (AML) cell line MV-4-11. T cells and tumor cells were immediately centrifuged and then resuspended in 200 ul serial dilutions of rapamycin prepared in heparinized human whole blood from healthy human volunteers or mouse whole blood from NSG mice. Co-cultures were incubated for 24 hours at 37° C. and then centrifuged to isolate plasma, which was used for assessment of IFNγ release using the Meso Scale Discovery (MSD) cytokine assay.

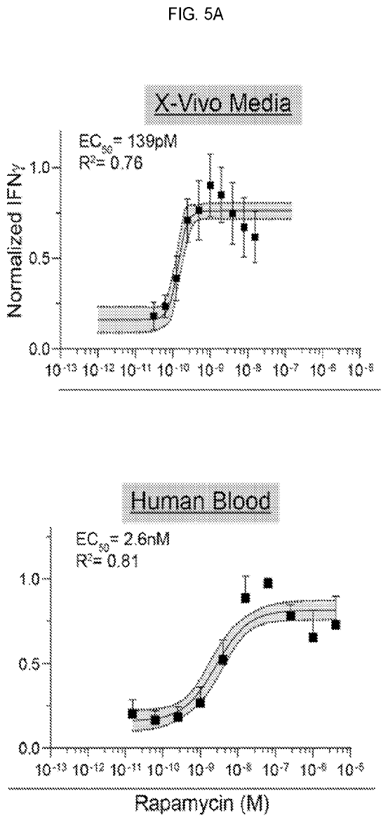

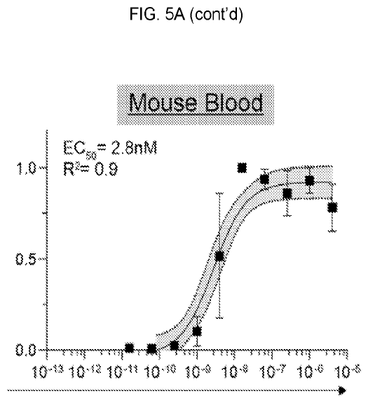

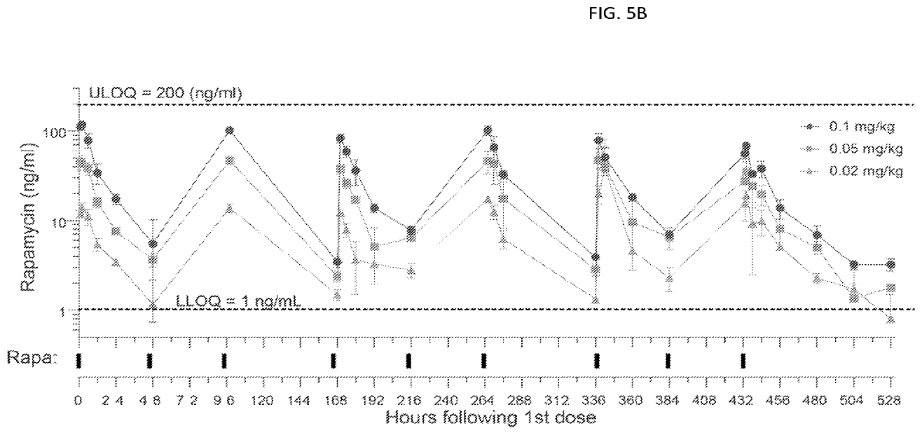

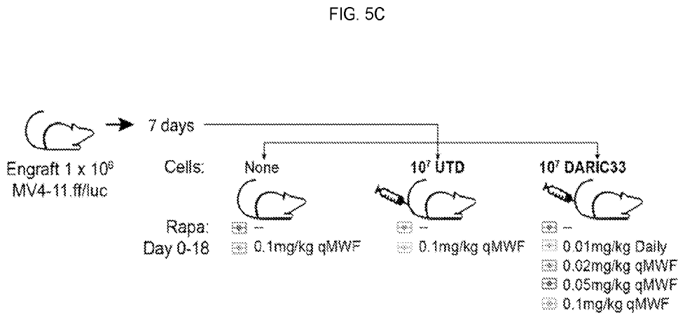

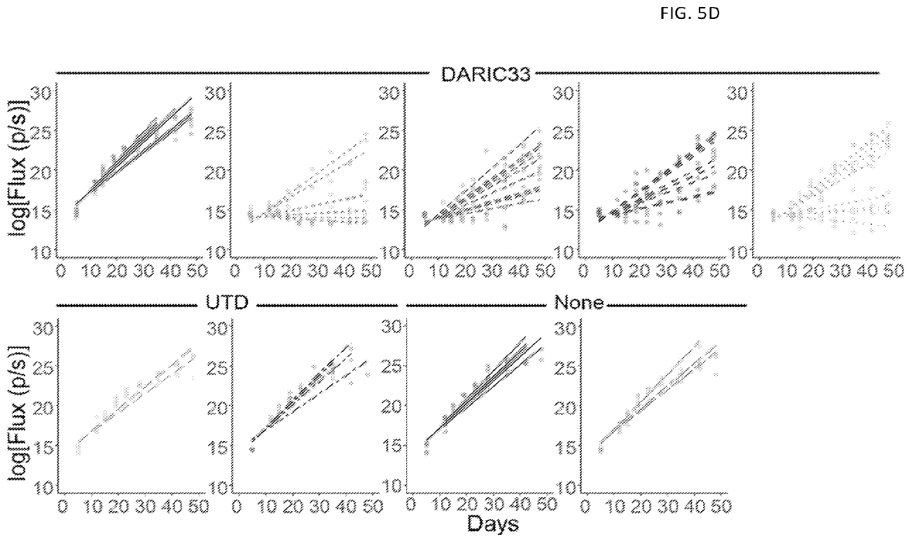

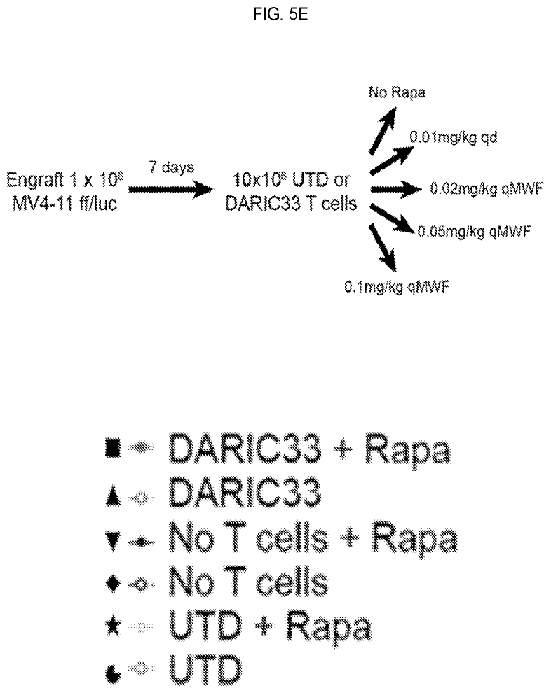

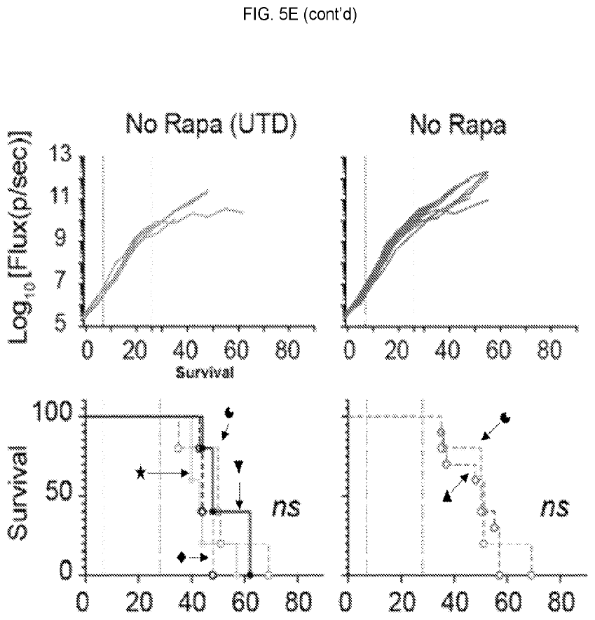

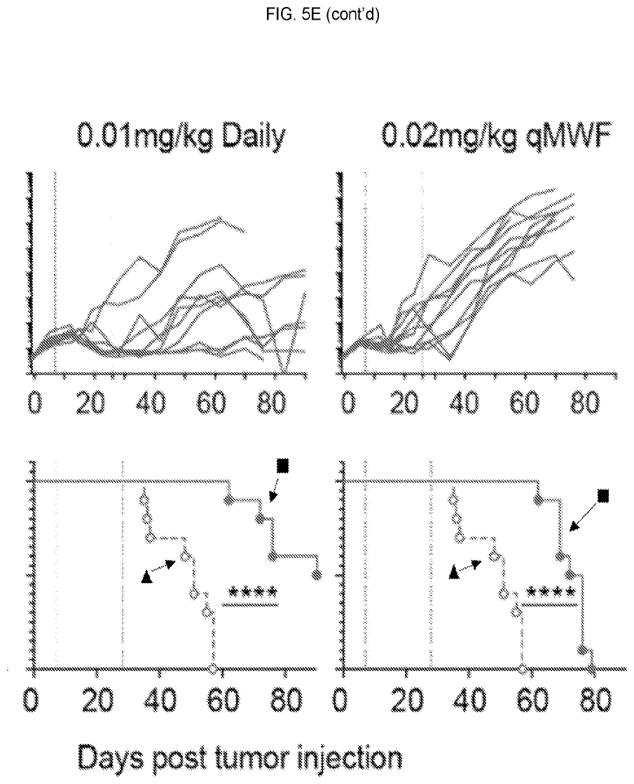

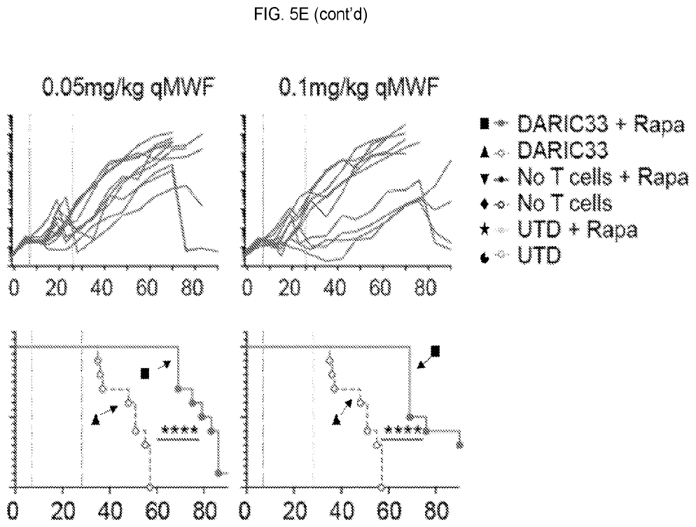

IFNγ values were used to calculate the rapamycin EC50 of IFNγ release by DARIC33 T cells. For each of the 6 total T cell and whole blood donor combinations across 2 experiments, replicates were averaged, and values were individually normalized such that the maximal IFNγ level was set to 1. Normalized data points were then plotted and fit to a three parameter dose-response curve, assuming a Hill slope of 1.0, to calculate EC50 using GraphPad Prism software. The resulting rapamycin EC50 was calculated at 2.6 nM with a range of 1.5-4.3 nM, representing the rapamycin concentration at which the IFNγ production by DARIC33 T cells co-cultured with tumor target cells was at half of its maximal value (FIGS. 5A-5E). The EC50 is consistent between activation by CD33+ cells present in healthy human blood, and human blood with exogenous CD33+ target cells added. Similar data transformations were performed for IFN^{γ} values obtained from T cell:tumor co-cultures in mouse blood. The calculated EC50 was 2.8 nM from 3 PBMC donor derived T cells with a range of 1.4-4.1 nM.

Rapamycin dependent increases in IFNγ release were similar across samples (IFNγ release, human blood, EC50=2.6 nM vs mouse blood EC50=2.8 nM), as well as, among human T cell donors (EC50 range of 1.5 nM-6.3 nM across three T cell donors and two blood donors, examined in duplicate, n=12 total). See FIG. 5A. These data define a target range of whole blood rapamycin concentrations capable of heterodimerizing the DARIC33.

Next measured was rapamycin exposure following single and repeat administrations in tumor-naive mice using a LC MS/MS quantitative whole blood assay. (J Chromatogr B Biomed Sci Appl, 1998 Nov. 6; 718(2)251-7). Concentrations of rapamycin in whole blood obtained during administration of various rapamycin doses 3 times weekly are shown, along with the timing of IP rapamycin injections, bars, below (see FIG. 5B). Upper limit of quantitation (ULOQ=200 ng/mL) and lower limit of quantitation (LLOQ=1 ng/mL) are indicated. Blood concentrations of rapamycin were generally dose proportional, peaked within 2 hours of intraperitoneal (IP) administration and decayed with an elimination half-time between 16 and 24 hours. Peak rapamycin concentrations ranged from 10 ng/mL at doses of 0.02 mg/kg to near 100 ng/mL at a dose of 0.1 mg/kg ((Table 1).

TABLE 1

| Dose | | | | | | | | AUC-T₀₋₄₈ |
|---|---|---|---|---|---|---|---|---|
| Level | Analysis | Tₘₐₓ | Tₘᵢₙ | T_{1/2} | Cₘₐₓ | Cₘᵢₙ | C_{48 h} | ng · |
| (mg/kg) | Day | (h) | (h) | (h) | (ng/mL) | (ng/mL) | (ng/mL) | h/mL) |
| 0.02 | 1 | 2 | 48 | 16.0 | 13.7 | 1.13 | 1.16 | 226 |
| 0.05 | 1 | 1 | 48 | 17.7 | 44.9 | 3.68 | 3.68 | 666 |
| 0.1 | 1 | 2 | 48 | 13.8 | 118.0 | 5.49 | 5.49 | 1480 |
| 0.02 | 19 | 2 | 96 | 25.1 | 19.0 | 0.80 | 2.27 | 318 |
| 0.05 | 19 | 2 | 72 | 21.8 | 34.9 | 1.35 | 5.03 | 621 |
| 0.1 | 19 | 2 | 96 | 24.3 | 68.3 | 3.22 | 6.91 | 1020 |

To determine the impact of various rapamycin dose levels and dosing schedules on anti-AML activity of SC-DARIC33 T cells, tumor bearing mice were treated with 10e7 (10 million) SC-DARIC33 T, or 10e6 UTD T cells followed by rapamycin (0.02 mg/kg qMWF, 0.05 mg/kg qMWF, 0.1 mg/kg qMWF and 0.01 mg/kg daily, all IP for 21 days, see FIG. 5C for schema). Among mice receiving dosing regimens predicted to be inactive (e.g. no treatment, rapamycin alone, UTD cells alone or followed by rapamycin, or SC-DARIC33 cells alone) tumor growth was not diminished (log[Flux]/day=0.26-0.27, FIG. 5E). In contrast, treatments predicted to be active (e.g., SC-DARIC33 product followed by rapamycin) exhibited lower rates of tumor growth, with the lowest rate observed among mice receiving 0.01 mg/kg rapamycin IP daily (log[Flux]/day=0.058). Tumor growth rates correlated with survival: while control mice developed tumor-associated symptoms near day 45, none of the mice receiving active treatment (DARIC33+rapamycin) exhibited signs of tumor progression at this time point. All rapamycin doses tested prolonged survival (p<0.001 log rank test): at the end of the 90-day observation period, treatment with SC-DARIC33 and 0.01 mg/kg rapamycin daily continued to control tumor outgrowth in 5 of 10 mice (FIG. 5E).

To define the minimum rapamycin concentrations associated with DARIC33 efficacy in tumor-bearing mice, concentrations of rapamycin were measured in the blood of mice treated with SC-DARIC33 T cells and the lowest rapamycin dose (0.01 mg/kg i.p. rapamycin daily) that exhibited in vivo anti-tumor activity. Blood samples obtained 2 hours after rapamycin administration on days 1 and 15, and 24 hours after rapamycin administration on day 20, contained 1.4 mg/mL to 3.3 ng/mL rapamycin (LC MS/MS quantitative whole blood assay, n=5 mice). Interestingly, while in vitro mouse and human whole blood assays showed similar rapamycin dependent DARIC33 activation, species differences were identified in rapamycin red blood cell (RBC) partitioning and plasma protein binding (PPB) ((Table 2 and Table 3).

TABLE 2
Red Blood Cell Partitioning of Rapamycin
in NSG Mouse Whole Blood (K₂EDTA)

| | Conc. in | | | | |
|---|---|---|---|---|---|
| | Spiked Plasma | | | | |
| | Conc. In | Ratio | | | |
| | Plasma from | Adjusted for | | % Bound | % Stability |
| Compound | Spiked Blood | Hematocrit | K_{p (RBC/PL)} | to RBC | in Plasma |
| Rapamycin | 0.55 | 1.06 | 0.0628 | 5.5% | 109.4% |
| (2 mM) | | | | | |

TABLE 3
Rapamycin (2 mM) Protein Binding in CD-1
mice, NSG mice, and Human Plasma (K₂EDTA)

| | % Protein | Fu Unbound | | % Stability |
|---|---|---|---|---|
| | Free | Fraction | % Protein Bound | in Matrix |
| Species | (Mean ± SD) | (Mean ± SD) | (Mean ± SD) | (2.5 hours) |
| CD-1 Mouse | 0.07 ± 0.20 | 0.0007 ± 0.0002 | 99.93 ± 0.02 | 104.23 |
| NSG Mouse | 0.06 ± 0.30 | 0.000553 ± 0.000341 | 99.94 ± 0.03 | 191.90 |
| (undiluted) | | | | |
| Human | 6.10 ± 8.20 | 0.061 ± 0.0082 | 93.90 ± 0.82 | 76.07 |

In humans, 94.5% of rapamycin is bound to RBCs while only 3.1% is found in plasma. In human plasma, rapamycin is highly protein bound (92%). In contrast, 5.5% RBC partitioning and greater than 99% PPB of rapamycin were observed in mice (Table 4),

TABLE 4
Rapamycin (2 mM) Protein Binding in 10% NSG Mouse Plasma (K₂EDTA)

| | | | Fu | Fu | | | |
|---|---|---|---|---|---|---|---|
| | | | (unbound | (Unbound | % Protein | | |
| | % Free | % Free | Fraction) | Fraction) | Bound | % Bound | % Stability |
| | Measured | Estimated | Measured | Estimated | Measured | Estimated | in |
| | in | in | in | in | in | in | Matrix |
| | Diluted | Undiluted | Diluted | Undiluted | Diluted | Undiluted | (2.5 |
| Species | Plasma | Plasma | Plasma | Plasma | Plasma | Plasma | Hours) |
| 10% | 6.82 ± | 0.73 ± | .0682 ± | .00728 ± | 93.18 ± | 99.27 ± | 75.1 |
| NSG | 1.04 | .1.10 | 0.0104 | 0.00111 | 1.04 | 0.11 | |
| Mouse | | | | | | | |

suggesting that these species-specific differences in rapamycin distribution result in similar concentrations of bioavailable rapamycin in vitro. Together, these data informed a target rapamycin trough blood concentration range of 1.5-3 ng/mL for DARIC heterodimerization in humans.

### Example 6: Determination of Whole Blood Rapamycin Concentration in Treated Mice

To determine the concentration of rapamycin present in mice that exhibited suppressed tumor growth, peripheral blood was obtained by retroorbital bleeding and frozen at −80° C. for analysis. Quantitative liquid chromatography-tandem mass spectrometry (LS-MS/MS) was used to determine concentrations of rapamycin present in whole blood samples. As shown in FIG. 6, samples obtained on day 1, 2 hours following rapamycin injection were found to contain 0.916+0.896 ng/mL of rapamycin. Samples obtained on day 15, 2 hours following rapamycin injection were found to contain 2.65±1.983 ng/mL rapamycin. These finding are consistent with accumulation of rapamycin in the animals; furthermore, the collection timing coincided with the peak levels of rapamycin observed in prior experiments. Samples obtained on day 21, 24 hours after administration of rapamycin were found to contain 0.56±0.771 ng/mL of rapamycin on average but was below the limit of quantitation in 3 of 5 animals. Together these data suggest that rapamycin doses that result in peak concentrations of 2.65±1.983 ng/mL and trough concentrations of 0.56±0.771 ng/mL are sufficient to activate DARIC33 T cells in mice to exert anti-AML tumor control.

### Example 7: In Silico Modeling of DARIC33 Rapamycin Response

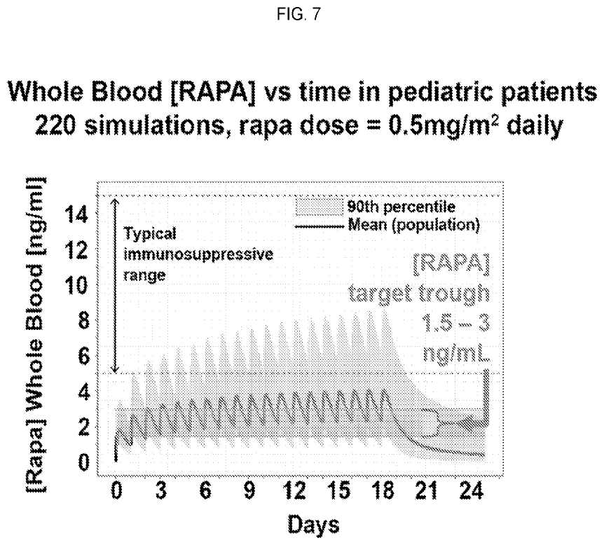

To determine the recommended Rapamycin (Sirolimus) starting dose in pediatric patients (also referred to as subjects), published pediatric patient Sirolimus whole blood exposures (Goyal et al, Biol Blood Marrow Transplant 2013, 569-575; Wu et al, CPT: Pharmacometrics & Systems Pharmacol 2012, 1, e17, doi:10.1038/psp.2012.18) were used to establish a population pharmacokinetic model. Several Sirolimus dose levels were simulated on a once daily dosing schedule for 19-21 days. Exposure profiles were generated showing geometric mean (dark blue line) and 10^{th}, 90^{th} percentiles (light blue shading) of expected Sirolimus concentrations (see FIG. 7). Typical immunosuppressive trough concentrations of Sirolimus are in the range of 5-15 ng/mL (red dashed lines). The simulation of a 0.50 mg/m² (for patients less than or equal to 1.5 m²) or 0.75 mg (for patients greater than or equal to 1.5 m²) Sirolimus dose, on a daily dosing schedule, provided the best exposure coverage while allowing the greatest number of patients to attain a constant Sirolimus exposure of 1.5-3 ng/ml (target trough concentration range, shaded green).

The target trough concentration range of Rapamycin was derived from the whole blood EC₅₀ for SC-DARIC33, which is 2.6 nM (equivalent to 2.6 ng/mL).

Furthermore, the target trough concentration range of Rapamycin was supported by analysis of Rapamycin whole blood concentrations associated with efficacy in tumor-bearing immune-compromised mice injected with SC-DARIC33, which indicated that the unbound Rapamycin concentrations in mice were below the unbound equivalent of the target trough concentration in pediatric patients.

In addition, the simulated Sirolimus dose of 0.50 mg/m² (for patients less than or equal to 1.5 m²) or 0.75 mg (for patients greater than or equal to 1.5 m²) Sirolimus dose, on a daily dosing schedule for 19 days, allowed for simulation of Sirolimus clearance following cessation of dosing that will enable the switching off of SC-DARIC33 activation.

### Example 8: Illustrative Clinical Protocol

Rapamycin (or Rapalog) dosing and target levels. Rapamycin will be initiated orally at a dose of 0.75 mg (for patients>1.5 m²) or 0.50 mg/m² (for patients 51.5 m²). Dosing should be adjusted to maintain a target trough blood level of 2 ng/mL within a target range of 1.5-3 ng/mL.

In the absence of evidence for DARIC activation, the target levels can be increased beyond 3, with a maximum target level of 9 ng/mL.

Initial Rapamycin Schedule. Beginning on Day +2 after SC-DARIC T cell product infusion, daily rapamycin will be administered to activate the SC-DARIC33. Rapamycin will be continued until day +21. Following the Day 28 bone marrow aspirate and/or biopsy, if the subject is in morphological remission with <1% disease by multiparameter flow, rapamycin will continue to be withheld. Subsequent rapamycin courses may be initiated per below after day +42.

If evaluation prior to day 42 demonstrates evidence of persistent leukemia at a level of >1% in the bone marrow, subsequent rapamycin courses can begin immediately at day 42.

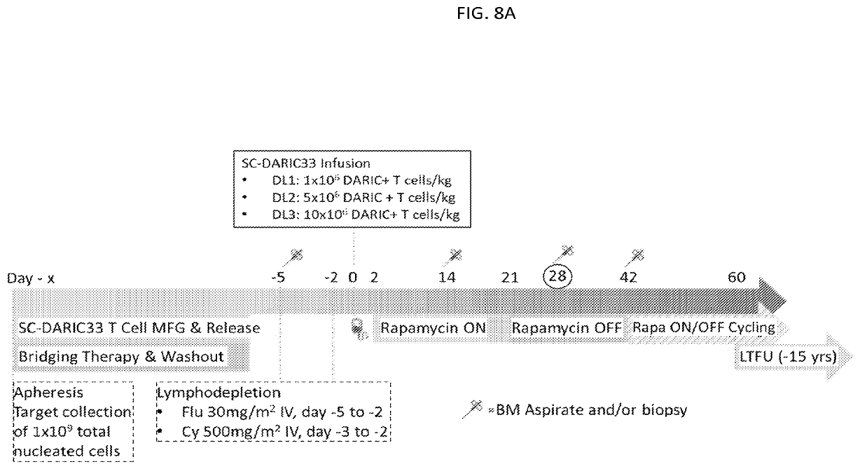

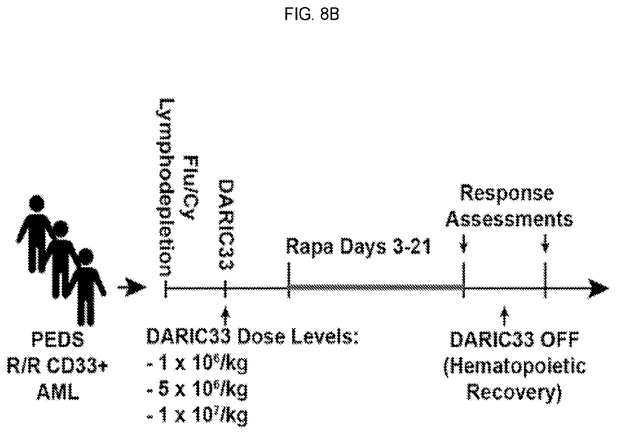

The protocol for infusion, rapamycin administration, and bone marrow aspirate and/or biopsy is illustrated in FIG. 8A. An alternative protocol wherein Rapamycin is administered on days 3-21 is shown in FIG. 8B.

Subsequent Rapamycin Courses (Table 5 and Table 6). For subjects who, according to the clinical judgement of the investigator, are felt to be deriving benefit from activation of the DARIC T cells with rapamycin, subsequent courses of rapamycin may be initiated at any time after day +42 post T cell infusion for patients in remission and immediately for patients with evidence of persistent leukemia, until a time in which there is no longer a benefit to DARIC T cell activity. The requirements to start rapamycin for each subsequent course include an absence of any Grade 3 or higher toxicity assessed as clinically significant and related to the DARIC T cells.

For subjects in an ongoing remission, subsequent courses of rapamycin would be administered at least 14 days from cessation of prior rapamycin administration and the patient must have an absolute phagocyte count (APC)>500 cells/μL.

TABLE 5
Subsequent Rapamycin Courses 1-6.

| | Day | | | | | |
|---|---|---|---|---|---|---|
| Timepoint | 0 | 3 | 7 | 10 | 14 | 21 |
| Visit Window | +/−2 days | | | | | |
| Physical Examination (PE) & | X | | X | | X | X |
| Vital Signs (VS)² | | | | | | |
| Chemistry¹ | X | | X | | | X |
| Hematology² | X | | X | | X | X |
| Sirolimus Trough Level | | X | X | X | X | X |
| Inflammatory markers³ | X | | X | | X | X |
| Cytokine Release Syndrome | Daily when Gr 1-4 CRS is occurring | | | | | |
| (CRS) labs⁵ | | | | | | |
| Correlative Studies | X | X | X | X | X | X |
| Peripheral Blood⁴ | | | | | | |

| | |
|---|---|
| Rapamycin | Continuous for 7 days |
| Concomitant medications | continuous |
| Adverse Events⁶ | continuous |

TABLE 6
Subsequent Rapamycin Courses 7+.

| | Day | | | |
|---|---|---|---|---|
| Timepoint | 0 | 7 | 14 | 21 |
| Visit Window | +/−3 days | | | |
| PE & VS² | X | | | |
| Chemistry¹ | X | | | |
| Hematology² | X | | | X |
| Sirolimus Trough Level | | X | | X |
| Inflammatory markers³ | X | | | |
| CRS labs⁵ | Daily when Gr 1-4 CRS is occurring | | | |
| Correlative Studies | X | X | X | X |
| Peripheral Blood⁴ | | | | |

| | |
|---|---|
| Rapamycin | Continuous for 7 days |

| | |
|---|---|
| Concomitant medications | continuous |
| Adverse Events⁶ | continuous |

For both Table 5 and Table 6, the superscripts indicate the following:
1. Serum sodium, potassium, chloride, bicarbonate, blood urea nitrogen (BUN)/urea, creatinine, total and conjugated bilirubin, alanine aminotransferase (ALT), lactic dehydrogenase (LDH), uric acid, calcium, magnesium, phosphorus
2. Complete blood count (CBC), differential and platelet count
3. Ferritin and CRP (C-reactive protein)
4. Refer to study specific lab manual for requirements of all correlative studies samples. Up to 1 mL/kg (not to exceed 40 mL) to Correlative Studies Laboratory (CSL) for correlative studies.
5. CRS labs include CRP, LDH, ferritin, D-Dimer, prothrombin time (PT), partial thromboplastin time (PTT), fibrinogen, and absolute lymphocyte count.
6. Record information on hospitalization, intensive care unit (ICU) admission, and absolute lymphocyte count (ANC) recovery following DARIC T cell infusion

Long Term Follow Up. Subjects will be followed for up to 15 years after DARIC T cell product infusion to capture delayed adverse events related to the use of lentivirally transduced T cells as required by the FDA and additional information including disease evaluation. If a donor for HCT has been identified and it is determined to be in the patient's best interest, then HCT preceded by conditioning can be initiated at physician discretion any time following DARIC T cell infusion. Information will be collected regarding HCT inclusive of conditioning regimen used and stem cell source as well as the date of HCT. In the subset of subjects with ongoing myeloid cell aplasia, infectious complications will also be collected. Follow up information may be provided by the subject's primary care physician.

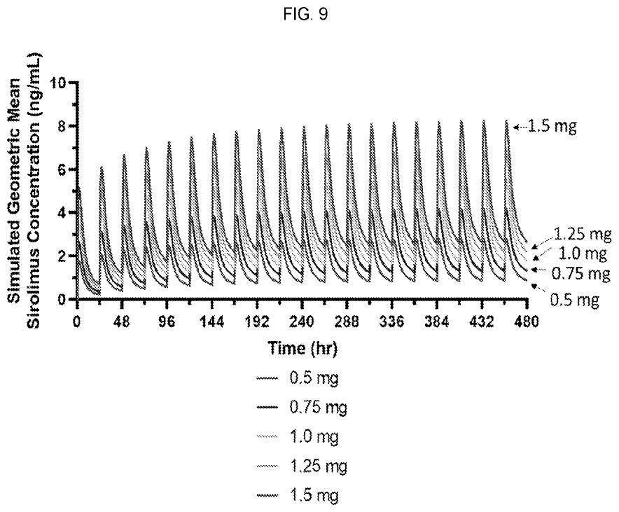

Example 9. To determine the recommended Rapamycin (Sirolimus) starting dose in adult patients (also referred to as subjects), published adult patient Sirolimus whole blood exposures (Wu et al, CPT Pharmacometrics Syst Pharmacol 2012, 1, e17) were used to establish a population pharmacokinetic model. Several Sirolimus dose levels (0.5 mg, 0.75 mg, 1.0 mg, 1.25 mg and 1.5 mg) were simulated on a once daily dosing schedule for 19-21 days (FIG. 9).

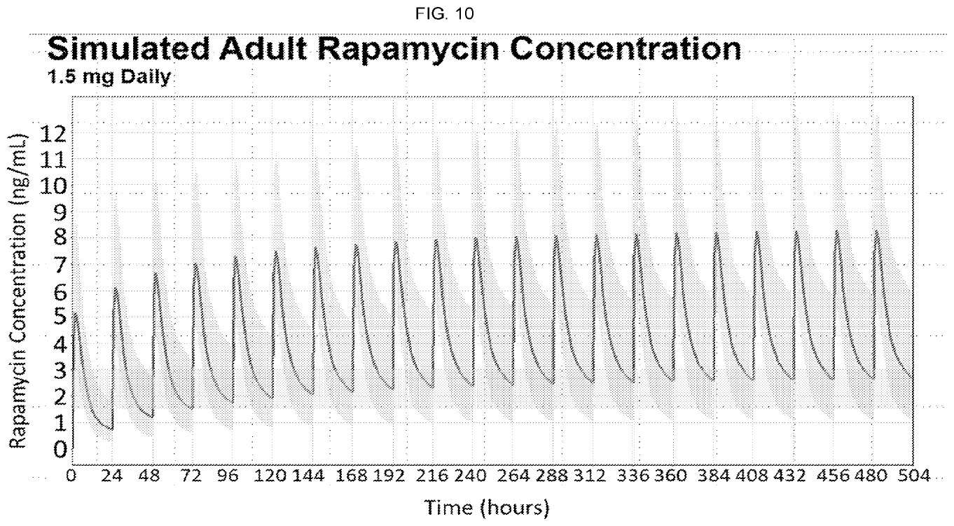

Exposure profiles for a starting dose of 1.5 mg daily were generated showing geometric mean (solid line) and 10^{th}, 90^{th} percentiles (shading) of expected Sirolimus concentrations (FIG. 10). An initial dose of 1.5 mg daily will enable a significant fraction of patients to attain target sirolimus concentrations of 1.5-3 ng/mL. Dosing adjustments can be made.

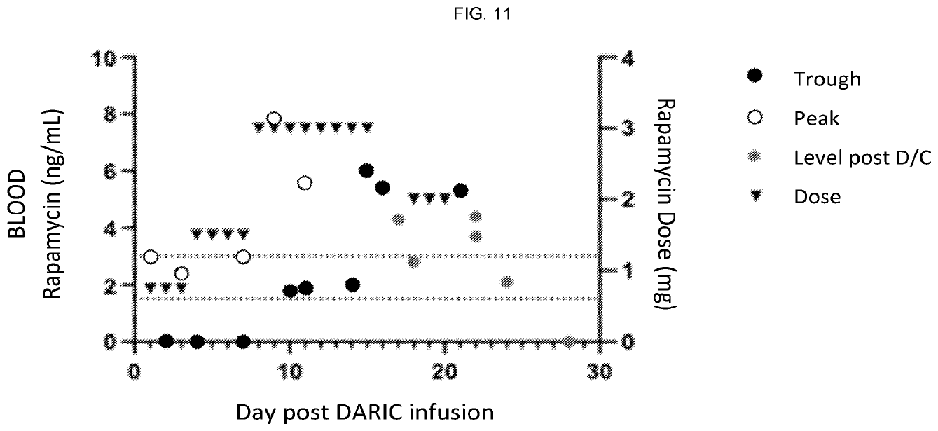

Example 10. Patient (>1.5 m²) pharmacokinetic (PK) data following rapamycin administration demonstrating the exposure relationship between dose and peak and trough levels, and dose adjustments to achieve the target range (FIG. 11). Rapamycin was initiated orally at a dose of 0.75 mg and peak and trough levels were monitored as described using the clinical LC-MS/MS assay. Peak exposure (2 h post administration) is within the target range of 1.5-3 ng/ml, however trough (24 h following Sirolimus treatment) falls below the limit of detection of the assay (<1 ng/ml). After three administrations of Sirolimus at the initial dose of 0.75 mg, the dose was adjusted to 1.5 mg, to reach the target trough within the bound of the horizontal dotted line. Following 4 doses trough values were still not within range and the dose adjusted again, to 3 mg, to achieve trough. Following 2 administrations of Sirolimus at 3 mg trough levels within range were detected and the Sirolimus dose was held. After eight administrations of Sirolimus at 3 mg trough levels were measured above range at which time Sirolimus was withheld for 2 days and the Sirolimus dose adjusted down to 2 mg and after 3 administrations at day 20 post-treatment Sirolimus was discontinued. Levels post Sirolimus discontinuation (D/C) were monitored with a final measurement at day 28 showing Sirolimus was undetectable. Importantly this data demonstrates how the dose can be adjusted following monitoring to achieve the target trough range of 1.5-3 ng/mL and supports the pre-clinical modeling.

### (x) SEQUENCES SUPPORTING THE DISCLOSURE

| SEQ ID | | |
|---|---|---|
| NO | NAME | SEQUENCE |
| 1 | Human CD33 | MPLLLLLPLLWAGALAMDPNFWLQVQESVTVQEGLCVLVPCTFFH |
| | | PIPYYDKNSPVHGYWFREGAIISRDSPVATNKLDQEVQEETQGRFR |
| | | LLGDPSRNNCSLSIVDARRRDNGSYFFRMERGSTKYSYKSPQLSV |
| | | HVTDLTHRPKILIPGTLEPGHSKNLTCSVSWACEQGTPPIFSWLSAA |
| | | PTSLGPRTTHSSVLIITPRPQDHGTNLTCQVKFAGAGVTTERTIQLN |
| | | VTYVPQNPTTGIFPGDGSGKQETRAGVVHGAIGGAGVTALLALCLC |
| | | LIFFIVKTHRRKAARTAVGRNDTHPTTGSASPKHQKKSKLHGPTET |
| | | SSCSGAAPTVEMDEELHYASLNFHGMNPSKDTSTEYSEVRTQ |
| 2 | anti-CD33 VHH- | QVTLRESGGGLVQAGGSLRLSCKGSGRAINTYAMGWFRQAPGKE |
| | 1 | REFVAAISWNGGHTRYADSVQGRFAISRDNADNTMYLQMNSLKPE |
| | | DTAVYHCAAYSDYHRIATMEADADSWGQGTQVTVSS |
| 3 | anti-CD33 VHH- | EVQLVESGGGEVQPGGSLRLSCAASGRTFSGYIMGWFRQAPGKE |
| | 2 | RELVARISGNNLSTEYAESVKGRFTISRDNAKNTLYLQMSSLRAED |
| | | TAVYYCAAEYDYSSGDFVYWGQGTLVTVKP |
| 4 | anti-CD33 VHH- | EVQLVESGGGEVQPGGSLRLSCAASGSGFSASLMSWHRQAPGK |
| | 3 | QRDLVASITRDGRANYVESVKGRFTISRDNAKNTLYLQMSSLRAED |
| | | TAVYYCHAYSFDYPIRSYWGQGTLVTVKP |
| 5 | anti-CD33 VHH- | EVQLVESGGGEVQPGGSLRLSCAASGFTFSSYAMGWFRQAPGKG |
| | 4 | REWVAAITTSGDTTYYAESVKGRFTISRDNAKNTVYLQMSSLRAED |
| | | TAVYYCAAHRGGGVIDYWGQGTLVTVKP |
| 6 | anti-CD33 VHH- | EVQLVESGGGEVQPGGSLRLSCAASGSIFSISIMGWYRQAPGKER |
| | 5 | ELVASTTSSGTTNYVESVKGRFTISRDNAKNTLYLQMSSLRAEDTA |
| | | VYYCHAYIATTTDRGYRGYWGQGTLVTVKP |
| 7 | anti-CD33 VHH- | QVQLVQSGGGLVQAGGSLRLSCAASGSISSYNVVGWYRQLSGNE |
| | 6 | RGGRTMVAQINAYGDTNYANAVVGRFTISRDDAKNTVYLHMSNLK |
| | | PEDTGVYYCNGQRMLENYTYRDQSWGQGTQVTVKP |
| 8 | anti-CD33 VHH- | QVQLVQSGGGLVQAGGSLTLSCAASRSSGIDVMGWYRQAPGKER |
| | 7 | ELVAEISGVGDTNYAASLADRFTVSRDNAKNTVYLQMKNLKPEDTA |
| | | VYYCNAHSFLDLVGAWGQGTQVTVKP |
| 9 | anti-CD33 VHH- | EVQLVQSGGGSVQVGGSLRLSCAASGSTLNIDHIGWYRQAPGKER |
| | 8 | ELVGVISSGAGPNYADSVKGRFTISRDNAKNTVYLQMNSLKPEDTA |
| | | VYNCNAWIDYGSGLPQNYWGQGTQVTVKP |
| 10 | anti-CD33 VHH- | EVQLVESGGGEVQPGGSLRLSCAASRSSGIDVMGWYRQAPGKER |
| | 9 | ELVAEISGVGDTNYAASLADRFTVSRDNAKNTVYLQMSSLRAEDTA |
| | | VYYCNAHSFLDLVGAWGQGTLVTVKP |
| 11 | anti-CD33 VHH- | EVQLVESGGGEVQPGGSLRLSCAASGSTLNIDHIGWYRQAPGKER |
| | 10 | ELVGVISSGAGPNYAESVKGRFTISRDNAKNTVYLQMSSLRAEDTA |
| | | VYYCNAWIDYGSGLPQNYWGQGTLVTVKP |
| 12 | anti-CD33 VHH- | QVTLRESGGGLVQAGGSLRLSCKGSGRAINTYAMGWFRQAPGKE |
| | 1 | REFVAAISWNGGHTRYADSVQGRFAISRDNADNTMYLQMNSLKPE |
| | | DTAVYHCAAYSDYHRIATMEADADSWGQGTQVTVXX |
| 13 | anti-CD33 VHH- | EVQLVESGGGEVQPGGSLRLSCAASGRTFSGYIMGWFRQAPGKE |
| | 2 | RELVARISGNNLSTEYAESVKGRFTISRDNAKNTLYLQMSSLRAED |
| | | TAVYYCAAEYDYSSGDFVYWGQGTLVTVXX |
| 14 | anti-CD33 VHH- | EVQLVESGGGEVQPGGSLRLSCAASGSGFSASLMSWHRQAPGK |
| | 3 | QRDLVASITRDGRANYVESVKGRFTISRDNAKNTLYLQMSSLRAED |
| | | TAVYYCHAYSFDYPIRSYWGQGTLVTVXX |
| 15 | anti-CD33 VHH- | EVQLVESGGGEVQPGGSLRLSCAASGFTFSSYAMGWFRQAPGKG |
| | 4 | REWVAAITTSGDTTYYAESVKGRFTISRDNAKNTVYLQMSSLRAED |
| | | TAVYYCAAHRGGGVIDYWGQGTLVTVXX |
| 16 | anti-CD33 VHH- | EVQLVESGGGEVQPGGSLRLSCAASGSIFSISIMGWYRQAPGKER |
| | 5 | ELVASTTSSGTTNYVESVKGRFTISRDNAKNTLYLQMSSLRAEDTA |
| | | VYYCHAYIATTTDRGYRGYWGQGTLVTVXX |
| 17 | anti-CD33 VHH- | QVQLVQSGGGLVQAGGSLRLSCAASGSISSYNVVGWYRQLSGNE |
| | 6 | RGGRTMVAQINAYGDTNYANAVVGRFTISRDDAKNTVYLHMSNLK |
| | | PEDTGVYYCNGQRMLENYTYRDQSWGQGTQVTVXX |
| 18 | anti-CD33 VHH- | QVQLVQSGGGLVQAGGSLTLSCAASRSSGIDVMGWYRQAPGKER |
| | 7 | ELVAEISGVGDTNYAASLADRFTVSRDNAKNTVYLQMKNLKPEDTA |
| | | VYYCNAHSFLDLVGAWGQGTQVTVXX |
| 19 | anti-CD33 VHH- | EVQLVQSGGGSVQVGGSLRLSCAASGSTLNIDHIGWYRQAPGKER |
| | 8 | ELVGVISSGAGPNYADSVKGRFTISRDNAKNTVYLQMNSLKPEDTA |
| | | VYNCNAWIDYGSGLPQNYWGQGTQVTVXX |
| 20 | anti-CD33 VHH- | EVQLVESGGGEVQPGGSLRLSCAASRSSGIDVMGWYRQAPGKER |
| | 9 | ELVAEISGVGDTNYAASLADRFTVSRDNAKNTVYLQMSSLRAEDTA |
| | | VYYCNAHSFLDLVGAWGQGTLVTVXX |
| 21 | anti-CD33 VHH- | EVQLVESGGGEVQPGGSLRLSCAASGSTLNIDHIGWYRQAPGKER |
| | 10 | ELVGVISSGAGPNYAESVKGRFTISRDNAKNTVYLQMSSLRAEDTA |
| | | VYYCNAWIDYGSGLPQNYWGQGTLVTVXX |
| 22 | anti-CD33.1 | METDTLLLWVLLLWVPGSTGQVTLRESGGGLVQAGGSLRLSCKGS |
| | DARIC binding | GRAINTYAMGWFRQAPGKEREFVAAISWNGGHTRYADSVQGRFAI |
| | domain | SRDNADNTMYLQMNSLKPEDTAVYHCAAYSDYHRIATMEADADS |
| | | WGQGTQVTVSSGGGGSGVQVETISPGDGRTFPKRGQTCVVHYT |
| | | GMLEDGKKFDSSRDRNKPFKFMLGKQEVIRGWEEGVAQMSVGQ |
| | | RAKLTISPDYAYGATGHPGIIPPHATLVFDVELLKLEGGRMALIVLGG |
| | | VAGLLLFIGLGIFFCVRCRHRRRQ |
| 23 | anti-CD33.2 | METDTLLLWVLLLWVPGSTGEVQLVESGGGEVQPGGSLRLSCAAS |
| | DARIC binding | GRTFSGYIMGWFRQAPGKERELVARISGNNLSTEYAESVKGRFTIS |
| | domain | RDNAKNTLYLQMSSLRAEDTAVYYCAAEYDYSSGDFVYWGQGTL |
| | | VTVKPGGGGSGVQVETISPGDGRTFPKRGQTCVVHYTGMLEDGK |
| | | KFDSSRDRNKPFKFMLGKQEVIRGWEEGVAQMSVGQRAKLTISPD |
| | | YAYGATGHPGIIPPHATLVFDVELLKLEGGRMALIVLGGVAGLLLFIG |
| | | LGIFFCVRCRHRRRQ |
| 24 | anti-CD33.3 | METDTLLLWVLLLWVPGSTGEVQLVESGGGEVQPGGSLRLSCAAS |
| | DARIC binding | GSGFSASLMSWHRQAPGKQRDLVASITRDGRANYVESVKGRFTIS |
| | domain | RDNAKNTLYLQMSSLRAEDTAVYYCHAYSFDYPIRSYWGQGTLVT |
| | | VKPGGGGSGVQVETISPGDGRTFPKRGQTCVVHYTGMLEDGKKF |
| | | DSSRDRNKPFKFMLGKQEVIRGWEEGVAQMSVGQRAKLTISPDYA |
| | | YGATGHPGIIPPHATLVFDVELLKLEGGRMALIVLGGVAGLLLFIGLG |
| | | IFFCVRCRHRRRQ |
| 25 | anti-CD33.4 | METDTLLLWVLLLWVPGSTGEVQLVESGGGEVQPGGSLRLSCAAS |
| | DARIC binding | GFTFSSYAMGWFRQAPGKGREWVAAITTSGDTTYYAESVKGRFTI |
| | domain | SRDNAKNTVYLQMSSLRAEDTAVYYCAAHRGGGVIDYWGQGTLV |
| | | TVKPGGGGSGVQVETISPGDGRTFPKRGQTCVVHYTGMLEDGKK |
| | | FDSSRDRNKPFKFMLGKQEVIRGWEEGVAQMSVGQRAKLTISPDY |
| | | AYGATGHPGIIPPHATLVFDVELLKLEGGRMALIVLGGVAGLLLFIGL |
| | | GIFFCVRCRHRRRQ |
| 26 | anti-CD33.5 | METDTLLLWVLLLWVPGSTGEVQLVESGGGEVQPGGSLRLSCAAS |
| | DARIC binding | GSIFSISIMGWYRQAPGKERELVASTTSSGTTNYVESVKGRFTISRD |
| | domain | NAKNTLYLQMSSLRAEDTAVYYCHAYIATTTDRGYRGYWGQGTLV |
| | | TVKPGGGGSGVQVETISPGDGRTFPKRGQTCVVHYTGMLEDGKK |
| | | FDSSRDRNKPFKFMLGKQEVIRGWEEGVAQMSVGQRAKLTISPDY |
| | | AYGATGHPGIIPPHATLVFDVELLKLEGGRMALIVLGGVAGLLLFIGL |
| | | GIFFCVRCRHRRRQ |
| 27 | anti-CD33.6 | METDTLLLWVLLLWVPGSTGQVQLVQSGGGLVQAGGSLRLSCAA |
| | DARIC binding | SGSISSYNVVGWYRQLSGNERGGRTMVAQINAYGDTNYANAVVG |
| | domain | RFTISRDDAKNTVYLHMSNLKPEDTGVYYCNGQRMLENYTYRDQS |
| | | WGQGTQVTVKPGGGGSGVQVETISPGDGRTFPKRGQTCVVHYT |
| | | GMLEDGKKFDSSRDRNKPFKFMLGKQEVIRGWEEGVAQMSVGQ |
| | | RAKLTISPDYAYGATGHPGIIPPHATLVFDVELLKLEGGRMALIVLGG |
| | | VAGLLLFIGLGIFFCVRCRHRRRQ |
| 28 | anti-CD33.7 | METDTLLLWVLLLWVPGSTGQVQLVQSGGGLVQAGGSLTLSCAAS |
| | DARIC binding | RSSGIDVMGWYRQAPGKERELVAEISGVGDTNYAASLADRFTVSR |
| | domain | DNAKNTVYLQMKNLKPEDTAVYYCNAHSFLDLVGAWGQGTQVTV |
| | | KPGGGGSGVQVETISPGDGRTFPKRGQTCVVHYTGMLEDGKKFD |
| | | SSRDRNKPFKFMLGKQEVIRGWEEGVAQMSVGQRAKLTISPDYAY |
| | | GATGHPGIIPPHATLVFDVELLKLEGGRMALIVLGGVAGLLLFIGLGI |
| | | FFCVRCRHRRRQ |
| 29 | anti-CD33.8 | METDTLLLWVLLLWVPGSTGEVQLVQSGGGSVQVGGSLRLSCAA |
| | DARIC binding | SGSTLNIDHIGWYRQAPGKERELVGVISSGAGPNYADSVKGRFTIS |
| | domain | RDNAKNTVYLQMNSLKPEDTAVYNCNAWIDYGSGLPQNYWGQGT |
| | | QVTVKPGGGGSGVQVETISPGDGRTFPKRGQTCVVHYTGMLEDG |
| | | KKFDSSRDRNKPFKFMLGKQEVIRGWEEGVAQMSVGQRAKLTISP |
| | | DYAYGATGHPGIIPPHATLVFDVELLKLEGGRMALIVLGGVAGLLLFI |
| | | GLGIFFCVRCRHRRRQ |
| 30 | anti-CD33.9 | METDTLLLWVLLLWVPGSTGEVQLVESGGGEVQPGGSLRLSCAAS |
| | DARIC binding | RSSGIDVMGWYRQAPGKERELVAEISGVGDTNYAASLADRFTVSR |
| | domain | DNAKNTVYLQMSSLRAEDTAVYYCNAHSFLDLVGAWGQGTLVTVK |
| | | PGGGGSGVQVETISPGDGRTFPKRGQTCVVHYTGMLEDGKKFDS |
| | | SRDRNKPFKFMLGKQEVIRGWEEGVAQMSVGQRAKLTISPDYAYG |
| | | ATGHPGIIPPHATLVFDVELLKLEGGRMALIVLGGVAGLLLFIGLGIFF |
| | | CVRCRHRRRQ |
| 31 | anti-CD33.10 | METDTLLLWVLLLWVPGSTGEVQLVESGGGEVQPGGSLRLSCAAS |
| | DARIC binding | GSTLNIDHIGWYRQAPGKERELVGVISSGAGPNYAESVKGRFTISR |
| | domain | DNAKNTVYLQMSSLRAEDTAVYYCNAWIDYGSGLPQNYWGQGTL |
| | | VTVKPGGGGSGVQVETISPGDGRTFPKRGQTCVVHYTGMLEDGK |
| | | KFDSSRDRNKPFKFMLGKQEVIRGWEEGVAQMSVGQRAKLTISPD |
| | | YAYGATGHPGIIPPHATLVFDVELLKLEGGRMALIVLGGVAGLLLFIG |
| | | LGIFFCVRCRHRRRQ |
| 32 | anti-CD33.1 | MALPVTALLLPLALLLHAARPGSILWHEMWHEGLEEASRLYFGERN |
| | DARIC fusion | VKGMFEVLEPLHAMMERGPQTLKETSFNQAYGRDLMEAQEWCRK |
| | protein | YMKSGNVKDLLQAWDLYYHVFRRISKASAGTGSDIYIWAPLAGTCG |
| | | VLLLSLVITMHKRGRKKLLYIFKQPFMRPVQTTQEEDGCSCRFPEE |
| | | EEGGCELRVKFSRSADAPAYQQGQNQLYNELNLGRREEYDVLDK |
| | | RRGRDPEMGGKPRRKNPQEGLYNELQKDKMAEAYSEIGMKGERR |
| | | RGKGHDGLYQGLSTATKDTYDALHMQALPPRSGSGATNFSLLKQA |
| | | GDVEENPGPSMETDTLLLWVLLLWVPGSTGQVTLRESGGGLVQA |
| | | GGSLRLSCKGSGRAINTYAMGWFRQAPGKEREFVAAISWNGGHT |
| | | RYADSVQGRFAISRDNADNTMYLQMNSLKPEDTAVYHCAAYSDYH |
| | | RIATMEADADSWGQGTQVTVSSGGGGSGVQVETISPGDGRTFPK |
| | | RGQTCVVHYTGMLEDGKKFDSSRDRNKPFKFMLGKQEVIRGWEE |
| | | GVAQMSVGQRAKLTISPDYAYGATGHPGIIPPHATLVFDVELLKLEG |
| | | GRMALIVLGGVAGLLLFIGLGIFFCVRCRHRRRQ |
| 33 | anti-CD33.2 | MALPVTALLLPLALLLHAARPGSILWHEMWHEGLEEASRLYFGERN |
| | DARIC fusion | VKGMFEVLEPLHAMMERGPQTLKETSFNQAYGRDLMEAQEWCRK |
| | protein | YMKSGNVKDLLQAWDLYYHVFRRISKASAGTGSDIYIWAPLAGTCG |
| | | VLLLSLVITMHKRGRKKLLYIFKQPFMRPVQTTQEEDGCSCRFPEE |
| | | EEGGCELRVKFSRSADAPAYQQGQNQLYNELNLGRREEYDVLDK |
| | | RRGRDPEMGGKPRRKNPQEGLYNELQKDKMAEAYSEIGMKGERR |
| | | RGKGHDGLYQGLSTATKDTYDALHMQALPPRSGSGATNFSLLKQA |
| | | GDVEENPGPSMETDTLLLWVLLLWVPGSTGEVQLVESGGGEVQP |
| | | GGSLRLSCAASGRTFSGYIMGWFRQAPGKERELVARISGNNLSTE |
| | | YAESVKGRFTISRDNAKNTLYLQMSSLRAEDTAVYYCAAEYDYSSG |
| | | DFVYWGQGTLVTVKPGGGGSGVQVETISPGDGRTFPKRGQTCVV |
| | | HYTGMLEDGKKFDSSRDRNKPFKFMLGKQEVIRGWEEGVAQMSV |
| | | GQRAKLTISPDYAYGATGHPGIIPPHATLVFDVELLKLEGGRMALIVL |
| | | GGVAGLLLFIGLGIFFCVRCRHRRRQ |
| 34 | anti-CD33.3 | MALPVTALLLPLALLLHAARPGSILWHEMWHEGLEEASRLYFGERN |
| | DARIC fusion | VKGMFEVLEPLHAMMERGPQTLKETSFNQAYGRDLMEAQEWCRK |
| | protein | YMKSGNVKDLLQAWDLYYHVFRRISKASAGTGSDIYIWAPLAGTCG |
| | | VLLLSLVITMHKRGRKKLLYIFKQPFMRPVQTTQEEDGCSCRFPEE |
| | | EEGGCELRVKFSRSADAPAYQQGQNQLYNELNLGRREEYDVLDK |
| | | RRGRDPEMGGKPRRKNPQEGLYNELQKDKMAEAYSEIGMKGERR |
| | | RGKGHDGLYQGLSTATKDTYDALHMQALPPRSGSGATNFSLLKQA |
| | | GDVEENPGPSMETDTLLLWVLLLWVPGSTGEVQLVESGGGEVQP |
| | | GGSLRLSCAASGSGFSASLMSWHRQAPGKQRDLVASITRDGRAN |
| | | YVESVKGRFTISRDNAKNTLYLQMSSLRAEDTAVYYCHAYSFDYPI |
| | | RSYWGQGTLVTVKPGGGGSGVQVETISPGDGRTFPKRGQTCVVH |
| | | YTGMLEDGKKFDSSRDRNKPFKFMLGKQEVIRGWEEGVAQMSVG |
| | | QRAKLTISPDYAYGATGHPGIIPPHATLVFDVELLKLEGGRMALIVLG |
| | | GVAGLLLFIGLGIFFCVRCRHRRRQ |
| 35 | anti-CD33.4 | MALPVTALLLPLALLLHAARPGSILWHEMWHEGLEEASRLYFGERN |
| | DARIC fusion | VKGMFEVLEPLHAMMERGPQTLKETSFNQAYGRDLMEAQEWCRK |
| | protein | YMKSGNVKDLLQAWDLYYHVFRRISKASAGTGSDIYIWAPLAGTCG |
| | | VLLLSLVITMHKRGRKKLLYIFKQPFMRPVQTTQEEDGCSCRFPEE |
| | | EEGGCELRVKFSRSADAPAYQQGQNQLYNELNLGRREEYDVLDK |
| | | RRGRDPEMGGKPRRKNPQEGLYNELQKDKMAEAYSEIGMKGERR |
| | | RGKGHDGLYQGLSTATKDTYDALHMQALPPRSGSGATNFSLLKQA |
| | | GDVEENPGPSMETDTLLLWVLLLWVPGSTGEVQLVESGGGEVQP |
| | | GGSLRLSCAASGFTFSSYAMGWFRQAPGKGREWVAAITTSGDTT |
| | | YYAESVKGRFTISRDNAKNTVYLQMSSLRAEDTAVYYCAAHRGGG |
| | | VIDYWGQGTLVTVKPGGGGSGVQVETISPGDGRTFPKRGQTCVV |
| | | HYTGMLEDGKKFDSSRDRNKPFKFMLGKQEVIRGWEEGVAQMSV |
| | | GQRAKLTISPDYAYGATGHPGIIPPHATLVFDVELLKLEGGRMALIVL |
| | | GGVAGLLLFIGLGIFFCVRCRHRRRQ |
| 36 | anti-CD33.5 | MALPVTALLLPLALLLHAARPGSILWHEMWHEGLEEASRLYFGERN |
| | DARIC fusion | VKGMFEVLEPLHAMMERGPQTLKETSFNQAYGRDLMEAQEWQRK |
| | protein | YMKSGNVKDLLQAWDLYYHVFRRISKASAGTGSDIYIWAPLAGTCG |
| | | VLLLSLVITMHKRGRKKLLYIFKQPFMRPVQTTQEEDGCSCRFPEE |
| | | EEGGCELRVKFSRSADAPAYQQGQNQLYNELNLGRREEYDVLDK |
| | | RRGRDPEMGGKPRRKNPQEGLYNELQKDKMAEAYSEIGMKGERR |
| | | RGKGHDGLYQGLSTATKDTYDALHMQALPPRSGSGATNFSLLKQA |
| | | GDVEENPGPSMETDTLLLWVLLLWVPGSTGEVQLVESGGGEVQP |
| | | GGSLRLSCAASGSIFSISIMGWYRQAPGKERELVASTTSSGTTNYV |
| | | ESVKGRFTISRDNAKNTLYLQMSSLRAEDTAVYYCHAYIATTTDRG |
| | | YRGYWGQGTLVTVKPGGGGSGVQVETISPGDGRTFPKRGQTCVV |
| | | HYTGMLEDGKKFDSSRDRNKPFKFMLGKQEVIRGWEEGVAQMSV |
| | | GQRAKLTISPDYAYGATGHPGIIPPHATLVFDVELLKLEGGRMALIVL |
| | | GGVAGLLLFIGLGIFFCVRCRHRRRQ |
| 37 | anti-CD33.6 | MALPVTALLLPLALLLHAARPGSILWHEMWHEGLEEASRLYFGERN |
| | DARIC fusion | VKGMFEVLEPLHAMMERGPQTLKETSFNQAYGRDLMEAQEWCRK |
| | protein | YMKSGNVKDLLQAWDLYYHVFRRISKASAGTGSDIYIWAPLAGTCG |
| | | VLLLSLVITMHKRGRKKLLYIFKQPFMRPVQTTQEEDGCSCRFPEE |
| | | EEGGCELRVKFSRSADAPAYQQGQNQLYNELNLGRREEYDVLDK |
| | | RRGRDPEMGGKPRRKNPQEGLYNELQKDKMAEAYSEIGMKGERR |
| | | RGKGHDGLYQGLSTATKDTYDALHMQALPPRSGSGATNFSLLKQA |
| | | GDVEENPGPSMETDTLLLWVLLLWVPGSTGQVQLVQSGGGLVQA |
| | | GGSLRLSCAASGSISSYNVVGWYRQLSGNERGGRTMVAQINAYG |
| | | DTNYANAVVGRFTISRDDAKNTVYLHMSNLKPEDTGVYYCNGQRM |
| | | LENYTYRDQSWGQGTQVTVKPGGGGSGVQVETISPGDGRTFPKR |
| | | GQTCVVHYTGMLEDGKKFDSSRDRNKPFKFMLGKQEVIRGWEEG |
| | | VAQMSVGQRAKLTISPDYAYGATGHPGIIPPHATLVFDVELLKLEGG |
| | | RMALIVLGGVAGLLLFIGLGIFFCVRCRHRRRQ |
| 38 | anti-CD33.7 | MALPVTALLLPLALLLHAARPGSILWHEMWHEGLEEASRLYFGERN |
| | DARIC fusion | VKGMFEVLEPLHAMMERGPQTLKETSFNQAYGRDLMEAQEWCRK |
| | protein | YMKSGNVKDLLQAWDLYYHVFRRISKASAGTGSDIYIWAPLAGTCG |
| | | VLLLSLVITMHKRGRKKLLYIFKQPFMRPVQTTQEEDGCSCRFPEE |
| | | EEGGCELRVKFSRSADAPAYQQGQNQLYNELNLGRREEYDVLDK |
| | | RRGRDPEMGGKPRRKNPQEGLYNELQKDKMAEAYSEIGMKGERR |
| | | RGKGHDGLYQGLSTATKDTYDALHMQALPPRSGSGATNFSLLKQA |
| | | GDVEENPGPSMETDTLLLWVLLLWVPGSTGQVQLVQSGGGLVQA |
| | | GGSLTLSCAASRSSGIDVMGWYRQAPGKERELVAEISGVGDTNYA |
| | | ASLADRFTVSRDNAKNTVYLQMKNLKPEDTAVYYCNAHSFLDLVG |
| | | AWGQGTQVTVKPGGGGSGVQVETISPGDGRTFPKRGQTCVVHYT |
| | | GMLEDGKKFDSSRDRNKPFKFMLGKQEVIRGWEEGVAQMSVGQ |
| | | RAKLTISPDYAYGATGHPGIIPPHATLVFDVELLKLEGGRMALIVLGG |
| | | VAGLLLFIGLGIFFCVRCRHRRRQ |
| 39 | anti-CD33.8 | MALPVTALLLPLALLLHAARPGSILWHEMWHEGLEEASRLYFGERN |
| | DARIC fusion | VKGMFEVLEPLHAMMERGPQTLKETSFNQAYGRDLMEAQEWCRK |
| | protein | YMKSGNVKDLLQAWDLYYHVFRRISKASAGTGSDIYIWAPLAGTCG |
| | | VLLLSLVITMHKRGRKKLLYIFKQPFMRPVQTTQEEDGCSCRFPEE |
| | | EEGGCELRVKFSRSADAPAYQQGQNQLYNELNLGRREEYDVLDK |
| | | RRGRDPEMGGKPRRKNPQEGLYNELQKDKMAEAYSEIGMKGERR |
| | | RGKGHDGLYQGLSTATKDTYDALHMQALPPRSGSGATNFSLLKQA |
| | | GDVEENPGPSMETDTLLLWVLLLWVPGSTGEVQLVQSGGGSVQV |
| | | GGSLRLSCAASGSTLNIDHIGWYRQAPGKERELVGVISSGAGPNYA |
| | | DSVKGRFTISRDNAKNTVYLQMNSLKPEDTAVYNCNAWIDYGSGL |
| | | PQNYWGQGTQVTVKPGGGGSGVQVETISPGDGRTFPKRGQTCV |
| | | VHYTGMLEDGKKFDSSRDRNKPFKFMLGKQEVIRGWEEGVAQMS |
| | | VGQRAKLTISPDYAYGATGHPGIIPPHATLVFDVELLKLEGGRMALI |
| | | VLGGVAGLLLFIGLGIFFCVRCRHRRRQ |
| 40 | anti-CD33.9 | MALPVTALLLPLALLLHAARPGSILWHEMWHEGLEEASRLYFGERN |
| | DARIC fusion | VKGMFEVLEPLHAMMERGPQTLKETSFNQAYGRDLMEAQEWQRK |
| | protein | YMKSGNVKDLLQAWDLYYHVFRRISKASAGTGSDIYIWAPLAGTCG |
| | | VLLLSLVITMHKRGRKKLLYIFKQPFMRPVQTTQEEDGCSCRFPEE |
| | | EEGGCELRVKFSRSADAPAYQQGQNQLYNELNLGRREEYDVLDK |
| | | RRGRDPEMGGKPRRKNPQEGLYNELQKDKMAEAYSEIGMKGERR |
| | | RGKGHDGLYQGLSTATKDTYDALHMQALPPRSGSGATNFSLLKQA |
| | | GDVEENPGPSMETDTLLLWVLLLWVPGSTGEVQLVESGGGEVQP |
| | | GGSLRLSCAASRSSGIDVMGWYRQAPGKERELVAEISGVGDTNYA |
| | | ASLADRFTVSRDNAKNTVYLQMSSLRAEDTAVYYCNAHSFLDLVG |
| | | AWGQGTLVTVKPGGGGSGVQVETISPGDGRTFPKRGQTCVVHYT |
| | | GMLEDGKKFDSSRDRNKPFKFMLGKQEVIRGWEEGVAQMSVGQ |
| | | RAKLTISPDYAYGATGHPGIIPPHATLVFDVELLKLEGGRMALIVLGG |
| | | VAGLLLFIGLGIFFCVRCRHRRRQ |
| 41 | anti-CD33.10 | MALPVTALLLPLALLLHAARPGSILWHEMWHEGLEEASRLYFGERN |
| | DARIC fusion | VKGMFEVLEPLHAMMERGPQTLKETSFNQAYGRDLMEAQEWCRK |
| | protein | YMKSGNVKDLLQAWDLYYHVFRRISKASAGTGSDIYIWAPLAGTCG |
| | | VLLLSLVITMHKRGRKKLLYIFKQPFMRPVQTTQEEDGCSCRFPEE |
| | | EEGGCELRVKFSRSADAPAYQQGQNQLYNELNLGRREEYDVLDK |
| | | RRGRDPEMGGKPRRKNPQEGLYNELQKDKMAEAYSEIGMKGERR |
| | | RGKGHDGLYQGLSTATKDTYDALHMQALPPRSGSGATNFSLLKQA |
| | | GDVEENPGPSMETDTLLLWVLLLWVPGSTGEVQLVESGGGEVQP |
| | | GGSLRLSCAASGSTLNIDHIGWYRQAPGKERELVGVISSGAGPNYA |
| | | ESVKGRFTISRDNAKNTVYLQMSSLRAEDTAVYYCNAWIDYGSGL |
| | | PQNYWGQGTLVTVKPGGGGSGVQVETISPGDGRTFPKRGQTCVV |
| | | HYTGMLEDGKKFDSSRDRNKPFKFMLGKQEVIRGWEEGVAQMSV |
| | | GQRAKLTISPDYAYGATGHPGIIPPHATLVFDVELLKLEGGRMALIVL |
| | | GGVAGLLLFIGLGIFFCVRCRHRRRQ |
| 42 | anti-CD33 | MALPVTALLLPLALLLHAARPGSILWHEMWHEGLEEASRLYFGERN |
| | DARIC signaling | VKGMFEVLEPLHAMMERGPQTLKETSFNQAYGRDLMEAQEWQRK |
| | domain | YMKSGNVKDLLQAWDLYYHVFRRISKASAGTGSDIYIWAPLAGTCG |
| | | VLLLSLVITMHKRGRKKLLYIFKQPFMRPVQTTQEEDGCSCRFPEE |
| | | EEGGCELRVKFSRSADAPAYQQGQNQLYNELNLGRREEYDVLDK |
| | | RRGRDPEMGGKPRRKNPQEGLYNELQKDKMAEAYSEIGMKGERR |
| | | RGKGHDGLYQGLSTATKDTYDALHMQALPPR |
| 43 | anti-CLL 1 VHH- | EVQLVESGGGEVQPGGSLRLSCAASGFLFSIYDMNWYRQAPGKE |
| | 1 | REWVAGITNNGYSTAYAESVKGRFTISRDNAKNTIYLQMSSLRAED |
| | | TAVYYCHADLTKAYDVEYAWGQGTLVTVKP |
| 44 | anti-CLL1 VHH- | EVQLVESGGGEVQPGGSLRLSCAASGFTFSVYDMNWYRQAPGKE |
| | 2 | REWVSGISNNGFSTAYAESVKGRFTISRDNAKNTLYLQMSSLRAED |
| | | TAVYYCNLDVHGRVGAQGDYWGQGTLVTVKP |
| 45 | anti-CLL1 VHH- | EVQLVESGGGEVQPGGSLRLSCAASGFTFDDYAIGWFRQAPGKE |
| | 3 | REGVSTISSSDGSTYYAESVKGRFTISRDNAKNTVYLQMSSLRAED |
| | | TAVYYCALREGLPLLLDTLWRQPVEYDYWGQGTQVTVKP |
| 46 | anti-CLL1 VHH- | EVQLVESGGGEVQPGGSLRLSCAASGLLFSIYDMNWYRQAPGKE |
| | 4 | REWVAGITNNGYSTAYAESVKGRFTISRDNAKNTVYLQMSSLRAE |
| | | DTAVYYCHTDEWGREYWGQGTLVTVKP |
| 47 | anti-CLL1 VHH- | EVQLVESGGGEVQPGGSLRLSCAASGRTFEYDDMVWFRQAPGKE |
| | 5 | REEVAGISWSGGSIYYAESVKGRFSISRDNAKNTVYLEMNRLKPED |
| | | TAVYYCNVGSYWGQGTLVTVKP |
| 48 | anti-CLL1 VHH- | EVQLVESGGGEVQPGGSLRLSCAASGFTFRINDMGWYRQAPGKE |
| | 6 | CEMVAGISNNGFSTAYAESVKGRFTISRDNAKNTVYLQMSSLRAED |
| | | TAVYYCHRTDEGAAHACSRGQGTLVTVKP |
| 49 | anti-CLL1 VHH- | EVQLVESGGGEVQPGGSLRLSCAASGFLFSIYDMNWYRQAPGKE |
| | 1 | REWVAGITNNGYSTAYAESVKGRFTISRDNAKNTIYLQMSSLRAED |
| | | TAVYYCHADLTKAYDVEYAWGQGTLVTVXX |
| 50 | anti-CLL1 VHH- | EVQLVESGGGEVQPGGSLRLSCAASGFTFSVYDMNWYRQAPGKE |
| | 2 | REWVSGISNNGFSTAYAESVKGRFTISRDNAKNTLYLQMSSLRAED |
| | | TAVYYCNLDVHGRVGAQGDYWGQGTLVTVXX |
| 51 | anti-CLL1 VHH- | EVQLVESGGGEVQPGGSLRLSCAASGFTFDDYAIGWFRQAPGKE |
| | 3 | REGVSTISSSDGSTYYAESVKGRFTISRDNAKNTVYLQMSSLRAED |
| | | TAVYYCALREGLPLLLDTLWRQPVEYDYWGQGTQVTVXX |
| 52 | anti-CLL1 VHH- | EVQLVESGGGEVQPGGSLRLSCAASGLLFSIYDMNWYRQAPGKE |
| | 4 | REWVAGITNNGYSTAYAESVKGRFTISRDNAKNTVYLQMSSLRAE |
| | | DTAVYYCHTDEWGREYWGQGTLVTVXX |
| 53 | anti-CLL1 VHH- | EVQLVESGGGEVQPGGSLRLSCAASGRTFEYDDMVWFRQAPGKE |
| | 5 | REEVAGISWSGGSIYYAESVKGRFSISRDNAKNTVYLEMNRLKPED |
| | | TAVYYCNVGSYWGQGTLVTVXX |
| 54 | anti-CLL1 VHH- | EVQLVESGGGEVQPGGSLRLSCAASGFTFRINDMGWYRQAPGKE |
| | 6 | CEMVAGISNNGFSTAYAESVKGRFTISRDNAKNTVYLQMSSLRAED |
| | | TAVYYCHRTDEGAAHACSRGQGTLVTVXX |

SEQ ID NO: 1 sets forth the amino acid sequence for full-length human CD33. SEQ ID NOs: 2-21 set forth the amino acid sequences for an anti-CD33 VHH domains. SEQ ID NOs: 22-31 set forth the amino acid sequences for anti-CD33 VHH DARIC binding domains. SEQ ID NOs: 32-41 set forth the amino acid sequences for anti-CD33 VHH DARIC fusion proteins that include the binding domain and intracellular signaling components separated by a self-cleavable 2A peptide. SEQ ID NO: 42 sets forth the amino acid sequence for an anti-CD33 VHH DARIC including an intracellular signaling component and multimerization domain, but no binding domain.

### (xi) CLOSING PARAGRAPHS

The nucleic acid and amino acid sequences provided herein are shown using letter abbreviations for nucleotide bases and amino acid residues, as defined in 37 C.F.R. § 1.822 and set forth in the tables in WIPO Standard ST.25 (1998), Appendix 2, Tables 1 and 3. Only one strand of each nucleic acid sequence is shown, but the complementary strand is understood as included in embodiments where it would be appropriate.

The terms “specific binding affinity” or “specifically binds” or “specific binding” or “specifically targets” as used herein, describe binding of one molecule to another at greater binding affinity than background binding. A binding domain (e.g., of a CAR including a binding domain) “specifically binds” to a target molecule if it binds to or associates with a target molecule with an affinity or Ka (i.e. an equilibrium association constant of a particular binding interaction with units of 1/M) of, for example, greater than or equal to 10⁵ M⁻¹. In particular embodiments, a binding domain (or CAR) binds to a target with a Ka greater than or equal to 10⁶ M⁻¹, 107 M⁻¹, 10⁸ M⁻¹, 109 M⁻¹, 10¹⁰ M⁻¹, 10¹¹ M⁻¹, 10¹² M⁻¹, or 10¹³ M⁻¹. “High affinity” binding domains refers to those binding domains with a Ka of at least 107 M⁻¹, at least 10⁸ M⁻¹, at least 109 M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (Kd) of a particular binding interaction with units of M (e.g., 10⁻⁵ M to 10⁻¹³ M, or less). Affinities of binding domains and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore T100, which is available from Biacore, Inc., Piscataway, N.J., or optical biosensor technology such as the EPIC system or EnSpire that are available from Corning and Perkin Elmer respectively (see also, e.g., Scatchard et al. (1949) Ann. N.Y. Acad. Sci. 51:660; U.S. Pat. Nos. 5,283,173; 5,468,614).

In particular embodiments, the affinity of specific binding is 2 times greater than background binding, 5 times greater than background binding, 10 times greater than background binding, 20 times greater than background binding, 50 times greater than background binding, 100 times greater than background binding, or 1000 times greater than background binding or more.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in cancer or a statistically significant increase in the activity of CAR-T cells by administration of rapamycin or analog thereof, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method comprising
Identifying a subject with cells expressing a dimerizing agent regulated immunomodulatory complex (DARIC), wherein the DARIC comprises:
a first fusion protein comprising a CD33 single domain variable heavy (VHH) binding domain, an FK506 binding protein (FKBP) multimerization domain, and a transmembrane domain, and
a second fusion protein comprising an FKBP-rapamycin binding (FRB) multimerization domain, a transmembrane domain, and an intracellular component; and
administering to the subject a course of rapamycin or an analog thereof that binds and is disposed between the multimerization domain of the first fusion protein and the multimerization domain of the second fusion protein
wherein the course:
results in a blood trough level of the rapamycin or the analog thereof of 1.5 ng/mL to 3 ng/mL;
begins 2 or 3 days after the subject has in vivo cells expressing the DARIC;
extends for 18, 19, or 20 days with daily administrations of the rapamycin or the analog thereof;
comprises a daily dose of 0.75 mg or more if the subject is greater than 1.5 m² or a daily dose of less than 0.75 mg if the subject is 1.5 m² or less; and
comprises a rest period at the end of the course wherein no rapamycin or analog thereof is administered to the subject.

2. A method comprising
Identifying a subject with cells expressing a dimerizing agent regulated immunomodulatory complex (DARIC), wherein the DARIC comprises:
a first fusion protein comprising an extracellular component, an FK506 binding protein (FKBP) or FKBP-rapamycin binding (FRB) multimerization domain or variant thereof, and a transmembrane domain, and
a second fusion protein comprising an FK506 binding protein (FKBP) or FKBP-rapamycin binding (FRB) multimerization domain or variant thereof, a transmembrane domain, and an intracellular component; and
administering to the subject a course of rapamycin or an analog thereof that binds and is disposed between the multimerization domain of the first fusion protein and the multimerization domain of the second fusion protein
wherein the course one or more of:
results in a blood trough level of the rapamycin or the analog thereof of 1.5 ng/mL to 3 ng/mL;
begins 0-4 days after the subject has in vivo cells expressing the DARIC;
extends for at least 14 days with daily administrations of the rapamycin or the analog thereof;
comprises a rest period at the end of the course wherein no rapamycin or analog thereof is administered to the subject.

3. A method comprising
Identifying a subject with cells expressing a dimerizing agent regulated immunomodulatory complex (DARIC), wherein the DARIC comprises:
a first fusion protein comprising an extracellular component, an FK506 binding protein (FKBP) or FKBP-rapamycin binding (FRB) multimerization domain or variant thereof, and a transmembrane domain, and
a second fusion protein comprising an FK506 binding protein (FKBP) or FKBP-rapamycin binding (FRB) multimerization domain or variant thereof, a transmembrane domain, and an intracellular component; and
administering to the subject a course of rapamycin or an analog thereof that binds and is disposed between the multimerization domain of the first fusion protein and the multimerization domain of the second fusion protein
wherein the course one or more of:
results in a blood trough level of the rapamycin or the analog thereof of 1.5 ng/mL to 3 ng/mL;
comprises a daily dose of 0.75 mg or more if the subject is greater than 1.5 m² or a daily dose of less than 0.75 mg, if the subject is 1.5 m² or less;
and
comprises a rest period at the end of the course wherein no rapamycin or analog thereof is administered to the subject.

4. A method comprising
Identifying a subject with cells expressing a dimerizing agent regulated immunomodulatory complex (DARIC), wherein the DARIC comprises:
a first fusion protein comprising a multimerization domain, and
a second fusion protein comprising a multimerization domain; and
administering to the subject a course of rapamycin or an analog thereof that binds and is disposed between the multimerization domain of the first fusion protein and the multimerization domain of the second fusion protein.

5. The method of claim 4, wherein the course results in a blood trough level of the rapamycin or the analog thereof of 1.0 ng/mL to 3 ng/mL.

6. The method of claim 4, wherein the course begins 0-4 days after the subject has in vivo cells expressing the DARIC.

7. The method of claim 4, wherein the course begins 2 or 3 days after the subject has in vivo cells expressing the DARIC.

8. The method of claim 4, wherein the course extends for at least 14 days.

9. The method of claim 4, wherein the course extends for 18, 19, 20, 21, or 22 days with daily administrations of the rapamycin or the analog thereof.

10. The method of claim 4, wherein the course comprises a daily dose of at least 0.75 mg if the subject has a body surface area of greater than 1.5 m² or a daily dose of less than 0.75 mg if the subject has a body surface area of 1.5 m² or less.

11. The method of claim 10, wherein the subject has a body surface area of greater than 1.5 m² and the daily dose is 0.75 mg-3.5 mg.

12. The method of claim 10, wherein the subject has a body surface area of 1.5 m² or less and the daily dose is 0.25 mg/m²-0.74 mg/m².

13. The method of claim 10, wherein the subject has a body surface area of 1.5 m² or less and the daily dose is 0.50 mg/m².

14. The method of claim 10, wherein the course comprises a rest period wherein no rapamycin or analog thereof is administered to the subject after the course of daily administrations.

15. The method of claim 14, wherein the rest period is 13, 14, or 15 days.

16. The method of claim 14, wherein the rest period is at least 13 days, at least 14 days, at least 15 days, at least 16 days, at least 17 days, at least 18 days, at least 19 days, at least 20 days, at least 21 days, or at least 22 days.

17. The method of claim 14, wherein the rest period is at least 14 days.

18. The method of claim 14, wherein the rest period is 14 days.

19. The method of claim 14, wherein the method further comprises administering a second course of the rapamycin or analog thereof after the rest period.

20. The method of claim 19, wherein the second course results in a blood trough level of the rapamycin or the analog thereof of 0.5 ng/mL to 3 ng/mL.

21. The method of claim 19, wherein the second course extends for at least 14 days.

22. The method of claim 19, wherein the second course extends for 18, 19, 20, 21, or 22 days with daily administrations of the rapamycin or the analog thereof.

23. The method of claim 19, wherein the second course comprises a daily dose of at least 0.75 mg if the subject has a body surface area of greater than 1.5 m² or a daily dose of less than 0.75 mg if the subject has a body surface area of 1.5 m² or less.

24. The method of claim 4, wherein
the multimerization domain of the first fusion protein comprises an FKBP-rapamycin binding (FRB) multimerization domain or variant thereof, and the multimerization domain of the second fusion protein comprises an FK506 binding protein (FKBP) multimerization domain or variant thereof or
the multimerization domain of the first fusion protein comprises an FK506 binding protein (FKBP) multimerization domain or a variant thereof, and the multimerization domain of the second fusion protein comprises an FKBP-rapamycin binding (FRB) multimerization domain or a variant thereof.

25. The method of claim 24, wherein the FKBP multimerization domain or the variant thereof is FKBP12.

26. The method of claim 24, wherein the FKBP multimerization domain or the variant thereof has the sequence as set forth in SEQ ID NO: 55 or 56 or has at least 90% sequence identity to the sequence as set forth in SEQ ID NO: 55 or 56.

27. The method of claim 24, wherein the FKBP multimerization domain or the variant thereof has the sequence as set forth in SEQ ID NO: 55 or 56 or has at least 95% sequence identity to the sequence as set forth in SEQ ID NO: 55 or 56.

28. The method of claim 24, wherein the FKBP multimerization domain or the variant thereof has the sequence as set forth in SEQ ID NO: 55 or 56 or has at least 98% sequence identity to the sequence as set forth in SEQ ID NO: 55 or 56.

29. The method of claim 24, wherein the FRB multimerization domain or the variant thereof is FRB T2098L.

30. The method of claim 24, wherein the FRB multimerization domain or the variant thereof has the sequence as set forth in SEQ ID NO: 57 or 58 or has at least 90% sequence identity to the sequence as set forth in SEQ ID NO: 57 or 58.

31. The method of claim 24, wherein the FRB multimerization domain or the variant thereof has the sequence as set forth in SEQ ID NO: 57 or 58 or has at least 95% sequence identity to the sequence as set forth in SEQ ID NO: 57 or 58.

32. The method of claim 24, wherein the FRB multimerization domain or the variant thereof has the sequence as set forth in SEQ ID NO: 57 or 58 or has at least 98% sequence identity to the sequence as set forth in SEQ ID NO: 57 or 58.

33. The method of claim 24, wherein the FRB multimerization domain and the FKBP multimerization domain localize extracellularly when the first fusion protein and the second fusion protein are expressed.

34. The method of claim 24, wherein the FRB multimerization domain and the FKBP multimerization domain localize intracellularly when the first fusion protein and the second fusion protein are expressed.

35. The method of claim 4, wherein the first fusion protein and/or the second fusion protein further comprise a binding domain.

36. The method of claim 35, wherein the binding domain binds a cancer antigen.

37. The method of claim 36, wherein the binding domain is a single domain variable heavy chain (VHH) or a single chain variable fragment (scFv).

38. The method of claim 36, wherein the binding domain comprises a receptor extracellular domain or ligand.

39. The method of claim 35, wherein the binding domain comprises a binding domain of a CD33 antibody.

40. The method of claim 39, wherein the binding domain of the CD33 antibody is a VHH.

41. The method of claim 40, wherein the VHH has the sequence as set forth in any one of SEQ ID NOs: 2, 3, 4, 5, 6, 7, 8, 9, 10, 11, 12, 13, 14, 15, 16, 17, 18, 19, 20, or 21.

42. The method of claim 40, wherein the VHH has at least 90% sequence identity to the sequence as set forth in any one of SEQ ID NOs: 2, 3, 4, 5, 6, 7, 8, 9, 10, 11, 12, 13, 14, 15, 16, 17, 18, 19, 20, or 21 and specifically binds CD33.

43. The method of claim 40, wherein the VHH has at least 95% sequence identity to the sequence as set forth in any one of SEQ ID NOs: 2, 3, 4, 5, 6, 7, 8, 9, 10, 11, 12, 13, 14, 15, 16, 17, 18, 19, 20, or 21 and specifically binds CD33.

44. The method of claim 40, wherein the VHH has at least 98% sequence identity to the sequence as set forth in any one of SEQ ID NOs: 2, 3, 4, 5, 6, 7, 8, 9, 10, 11, 12, 13, 14, 15, 16, 17, 18, 19, 20, or 21 and specifically binds CD33.

45. The method of claim 4, wherein the first fusion protein and/or the second fusion protein comprise a binding domain that binds CLL1.

46. The method of claim 45, wherein the binding domain that binds CLL1 has the sequence as set forth in any one of SEQ ID NOs: 43, 44, 45, 46, 47, 48, 49, 50, 51, 52, 53 or 54.

47. The method of claim 45, wherein the binding domain that binds CLL1 has at least 90% sequence identity to the sequence as set forth in any one of SEQ ID NOs: 43, 44, 45, 46, 47, 48, 49, 50, 51, 52, 53 or 54 and specifically binds CLL1.

48. The method of claim 45, wherein the binding domain that binds CLL1 has at least 95% sequence identity to the sequence as set forth in any one of SEQ ID NOs: 43, 44, 45, 46, 47, 48, 49, 50, 51, 52, 53 or 54 and specifically binds CLL1.

49. The method of claim 45, wherein the binding domain that binds CLL1 has at least 98% sequence identity to the sequence as set forth in any one of SEQ ID NOs: 43, 44, 45, 46, 47, 48, 49, 50, 51, 52, 53 or 54 and specifically binds CLL1.

50. The method of claim 4, wherein the first fusion protein and/or the second fusion protein further comprise an intracellular component.

51. The method of claim 50, wherein the intracellular component comprises an intracellular primary signaling domain.

52. The method of claim 51, wherein the intracellular primary signaling domain comprises CD3ζ or a fragment thereof.

53. The method of claim 50, wherein the intracellular component comprises a co-receptor domain.

54. The method of claim 50, wherein the intracellular component comprises a costimulatory domain.

55. The method of claim 54, wherein the costimulatory domain comprises Toll-like receptor 1 (TLR1), TLR2, TLR3, TLR4, TLR5, TLR6, TLR7, TLR8, TLR9, TLR10, caspase recruitment domain family member 11 (CARD11), CD2, CD7, CD27, CD28, CD30, CD40, CD54 (ICAM), CD83, CD94, CD134 (OX40), CD137 (4-1BB), CD278 (ICOS), DNAX-Activation Protein 10 (DAP10), Linker for activation of T-cells family member 1 (LAT), SH2 Domain-Containing Leukocyte Protein Of 76 kD (SLP76), T cell receptor associated transmembrane adaptor 1 (TRAT1), TNFR2, TNFRS14, TNFRS18, TNRFS25, zeta chain of T cell receptor associated protein kinase 70 (ZAP70), or a fragment or combination thereof.

56. The method of claim 54, wherein the costimulatory domain comprises CD137 (4-1BB) or a fragment or combination thereof.

57. The method of claim 4, wherein the first fusion protein and/or the second fusion protein further comprise a transmembrane domain.

58. The method of claim 57, wherein the transmembrane domain of the first fusion protein and/or the second fusion protein is a CD4 transmembrane domain or a CD8α transmembrane domain.

59. The method of claim 4, wherein the first fusion protein and/or the second fusion protein further comprises a spacer.

60. The method of claim 4, wherein:
(a) the first fusion protein comprises: an FRB multimerization domain or variant thereof; a CD8α transmembrane domain or a CD4 transmembrane domain; a CD137 co-stimulatory domain; and/or a CD3ζ primary signaling domain; and
(b) the second fusion protein comprises: a CD33 VHH that has an amino acid sequence as set forth in any one of SEQ ID NOs: 2-21; an FKBP multimerization domain or variant thereof; and a CD4 transmembrane domain or a CD8α transmembrane domain.

61. The method of claim 4, wherein the first fusion protein comprises a signal peptide, a CD8α transmembrane domain; a CD137 co-stimulatory domain; and a CD3ζ primary signaling domain.

62. The method of claim 4, wherein the second fusion protein comprises a signal peptide and a CD4 transmembrane domain.

63. The method of claim 4, wherein the first fusion protein and/or the second fusion protein has the sequence as set forth in any one of SEQ ID NOs: 32, 33, 34, 35, 36, 37, 38, 39, 40, or 41.

64. The method of claim 4, wherein the first fusion protein and/or the second fusion protein has at least 90% sequence identity to the sequence as set forth in any one of SEQ ID NOs: 32, 33, 34, 35, 36, 37, 38, 39, 40, or 41.

65. The method of claim 4, wherein the first fusion protein and/or the second fusion protein has the sequence as set forth in SEQ ID NO: 40.

66. The method of claim 4, wherein the first fusion protein and/or the second fusion protein has at least 90% sequence identity to the sequence as set forth in SEQ ID NO: 40.

67. The method of claim 4, wherein the first fusion protein and/or the second fusion protein has at least 95% sequence identity to the sequence as set forth in SEQ ID NO: 40.

68. The method of claim 4, wherein the first fusion protein and/or the second fusion protein has at least 98% sequence identity to the sequence as set forth in SEQ ID NO: 40.

69. The method of claim 4, wherein the rapamycin or the analog thereof comprises rapamycin, AP1903, AP20187, AP21967, everolimus, novolimus, pimecrolimus, ridaforolimus, tacrolimus, temsirolimus, umirolimus, or zotarolimus.

70. The method of claim 4, wherein at least a subset of the cells expressing the DARIC in the identified subject are hematopoietic cells.

71. The method of claim 4, wherein at least a subset of the cells expressing the DARIC in the identified subject are T cells.

72. The method of claim 4, wherein at least a subset of the cells expressing the DARIC in the identified subject are as T cell or γδ T cell.

73. The method of claim 4, wherein at least a subset of the cells expressing the DARIC in the identified subject are CD3+, CD4+, or CD8+ cells.

74. The method of claim 4, wherein at least a subset of the cells expressing the DARIC in the identified subject are immune effector cells, wherein at least a subset of the cells expressing the DARIC in the identified subject.

75. The method of claim 4, wherein at least a subset of the cells expressing the DARIC in the identified subject are cytotoxic T lymphocytes (CTLs), tumor infiltrating lymphocytes (TILs), or helper T cells.

76. The method of claim 4, wherein at least a subset of the cells expressing the DARIC in the identified subject are natural killer (NK) cells or natural killer T (NKT) cells.

77. The method of claim 4, wherein the cells were isolated from peripheral blood mononuclear cells, bone marrow, lymph nodes tissue, cord blood, thymus issue, tissue from a site of infection, ascites, pleural effusion, spleen tissue, or tumors.

78. The method of claim 77, wherein the cells were ex vivo manufactured to express the DARIC.

79. The method of claim 78, wherein the cells were administered to the subject at a dose of cells per weight of subject of from 1×10⁵ cells/kg to 2000×10⁶ cells/kg.

80. The method of claim 78, wherein the cells were administered to the subject at a dose of cells per weight of subject of from 1×10⁶ cells/kg to 1000×10⁶ cells/kg.

81. The method of claim 78, wherein the cells were administered to the subject at a dose of cells per weight of subject of from 1×10⁶ cells/kg to 100×10⁶ cells/kg.

82. The method of claim 78, wherein the cells were administered to the subject at a dose of cells per weight of subject of from 5×10⁶ cells/kg to 500×10⁶ cells/kg.

83. The method of claim 78, wherein the cells were administered to the subject at a dose of cells per weight of subject of from 10×10⁶ cells/kg to 1000×10⁶ cells/kg.

84. The method of claim 78, wherein the cells were administered to the subject at a dose of cells per weight of subject of from 1×10⁶ cells/kg to 2×10⁶ cells/kg.

85. The method of claim 78, wherein the cells were administered to the subject at a dose of cells per weight of subject of from 3×10⁶ cells/kg to 5×10⁶ cells/kg.

86. The method of claim 78, wherein the cells were administered to the subject at a dose of cells per weight of subject of from 7.5×10⁶ cells/kg to 15×10⁶ cells/kg.

87. The method of claim 78, wherein the dose of cells per weight of subject is 10×10⁶ cells/kg.

88. The method of claim 4, wherein the cells were modified in the subject in vivo to express the DARIC.

89. The method of claim 4, wherein the subject is a pediatric patient.

90. The method of claim 4, wherein the subject is no more than 28 years old.

91. The method of claim 4, wherein the subject is no more than 18 years old.

92. The method of claim 4, wherein the subject is from 18 to 28 years old.

93. The method of claim 4, wherein the subject is an adult patient.

94. The method of claim 4, wherein the subject is at least 18 years old.

95. The method of claim 4, wherein the subject has or is diagnosed with a cancer, infectious disease, autoimmune disease, inflammatory disease, an immunodeficiency, or a condition associated therewith.

96. The method of claim 4, wherein the subject has or is diagnosed with a solid cancer.

97. The method of claim 96, wherein the solid cancer comprises lung cancer, squamous cell carcinoma, colorectal cancer, pancreatic cancer, breast cancer, thyroid cancer, bladder cancer, cervical cancer, esophageal cancer, ovarian cancer, gastric cancer, endometrial cancer, or brain cancer.

98. The method of claim 97, wherein the lung cancer is a non-small cell lung carcinoma.

99. The method of claim 97, wherein the squamous cell carcinoma is head and neck squamous cell carcinoma.

100. The method of claim 97, wherein the brain cancer comprises gliomas, glioblastomas, or oligodendrogliomas.

101. The method of claim 4, wherein the subject has or is diagnosed with a hematological malignancy.

102. The method of claim 101, wherein the hematological malignancy is a leukemia, lymphoma, or multiple myeloma.

103. The method of claim 4, wherein the haematological malignancy is acute myelogenous leukemia (AML).

104. The method of claim 4, wherein the subject is lymphodepleted prior to administering the course.

105. The method of claim 104, wherein the lymphodepletion comprises administering a dose of Fludarabine and a dose of Cyclophosphamide.

106. The method of claim 105, wherein the Fludarabine is administered at a dose of 30 mg/m² IV once daily for 4 days, and the Cyclophosphamide is administered at a dose of 500 mg/m² IV once daily for 2 days.

107. The method of claim 105, wherein the Cyclophosphamide is administered on days 3 and 4 of Fludarabine administration.

108. The method of claim 104, wherein the lymphodepletion begins 7 days prior to administering the course.


SEQUENCE LISTING (59 sequences)

SEQ ID NO: 1 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 2 - protein, 125 aa, synthetic construct
QVTLRESGGGLVQAGGSLRLSCKGSGRAINTYAMGWFRQAPGKEREFVAAISWNGGHTRYADSVQGRFAISRDNADNTMYLQMNSLKPEDTAVYHCAAYSDYHRIATMEADADSWGQGTQVTVSS

SEQ ID NO: 3 - protein, 120 aa, synthetic construct
EVQLVESGGGEVQPGGSLRLSCAASGRTFSGYIMGWFRQAPGKERELVARISGNNLSTEYAESVKGRFTISRDNAKNTLYLQMSSLRAEDTAVYYCAAEYDYSSGDFVYWGQGTLVTVKP

SEQ ID NO: 4 - protein, 118 aa, synthetic construct
EVQLVESGGGEVQPGGSLRLSCAASGSGFSASLMSWHRQAPGKQRDLVASITRDGRANYVESVKGRFTISRDNAKNTLYLQMSSLRAEDTAVYYCHAYSFDYPIRSYWGQGTLVTVKP

SEQ ID NO: 5 - protein, 118 aa, synthetic construct
EVQLVESGGGEVQPGGSLRLSCAASGFTFSSYAMGWFRQAPGKGREWVAAITTSGDTTYYAESVKGRFTISRDNAKNTVYLQMSSLRAEDTAVYYCAAHRGGGVIDYWGQGTLVTVKP

SEQ ID NO: 6 - protein, 121 aa, synthetic construct
EVQLVESGGGEVQPGGSLRLSCAASGSIFSISIMGWYRQAPGKERELVASTTSSGTTNYVESVKGRFTISRDNAKNTLYLQMSSLRAEDTAVYYCHAYIATTTDRGYRGYWGQGTLVTVKP

SEQ ID NO: 7 - protein, 124 aa, synthetic construct
QVQLVQSGGGLVQAGGSLRLSCAASGSISSYNVVGWYRQLSGNERGGRTMVAQINAYGDTNYANAVVGRFTISRDDAKNTVYLHMSNLKPEDTGVYYCNGQRMLENYTYRDQSWGQGTQVTVKP

SEQ ID NO: 8 - protein, 116 aa, synthetic construct
QVQLVQSGGGLVQAGGSLTLSCAASRSSGIDVMGWYRQAPGKERELVAEISGVGDTNYAASLADRFTVSRDNAKNTVYLQMKNLKPEDTAVYYCNAHSFLDLVGAWGQGTQVTVKP

SEQ ID NO: 9 - protein, 120 aa, synthetic construct
EVQLVQSGGGSVQVGGSLRLSCAASGSTLNIDHIGWYRQAPGKERELVGVISSGAGPNYADSVKGRFTISRDNAKNTVYLQMNSLKPEDTAVYNCNAWIDYGSGLPQNYWGQGTQVTVKP

SEQ ID NO: 10 - protein, 116 aa, synthetic construct
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKP

SEQ ID NO: 11 - protein, 120 aa, synthetic construct
EVQLVESGGGEVQPGGSLRLSCAASGSTLNIDHIGWYRQAPGKERELVGVISSGAGPNYAESVKGRFTISRDNAKNTVYLQMSSLRAEDTAVYYCNAWIDYGSGLPQNYWGQGTLVTVKP

SEQ ID NO: 12 - protein, 125 aa, synthetic construct
QVTLRESGGGLVQAGGSLRLSCKGSGRAINTYAMGWFRQAPGKEREFVAAISWNGGHTRYADSVQGRFAISRDNADNTMYLQMNSLKPEDTAVYHCAAYSDYHRIATMEADADSWGQGTQVTVXX

SEQ ID NO: 13 - protein, 120 aa, synthetic construct
EVQLVESGGGEVQPGGSLRLSCAASGRTFSGYIMGWFRQAPGKERELVARISGNNLSTEYAESVKGRFTISRDNAKNTLYLQMSSLRAEDTAVYYCAAEYDYSSGDFVYWGQGTLVTVXX

SEQ ID NO: 14 - protein, 118 aa, synthetic construct
EVQLVESGGGEVQPGGSLRLSCAASGSGFSASLMSWHRQAPGKQRDLVASITRDGRANYVESVKGRFTISRDNAKNTLYLQMSSLRAEDTAVYYCHAYSFDYPIRSYWGQGTLVTVXX

SEQ ID NO: 15 - protein, 118 aa, synthetic construct
EVQLVESGGGEVQPGGSLRLSCAASGFTFSSYAMGWFRQAPGKGREWVAAITTSGDTTYYAESVKGRFTISRDNAKNTVYLQMSSLRAEDTAVYYCAAHRGGGVIDYWGQGTLVTVXX

SEQ ID NO: 16 - protein, 121 aa, synthetic construct
EVQLVESGGGEVQPGGSLRLSCAASGSIFSISIMGWYRQAPGKERELVASTTSSGTTNYVESVKGRFTISRDNAKNTLYLQMSSLRAEDTAVYYCHAYIATTTDRGYRGYWGQGTLVTVXX

SEQ ID NO: 17 - protein, 124 aa, synthetic construct
QVQLVQSGGGLVQAGGSLRLSCAASGSISSYNVVGWYRQLSGNERGGRTMVAQINAYGDTNYANAVVGRFTISRDDAKNTVYLHMSNLKPEDTGVYYCNGQRMLENYTYRDQSWGQGTQVTVXX

SEQ ID NO: 18 - protein, 116 aa, synthetic construct
QVQLVQSGGGLVQAGGSLTLSCAASRSSGIDVMGWYRQAPGKERELVAEISGVGDTNYAASLADRFTVSRDNAKNTVYLQMKNLKPEDTAVYYCNAHSFLDLVGAWGQGTQVTVXX

SEQ ID NO: 19 - protein, 120 aa, synthetic construct
EVQLVQSGGGSVQVGGSLRLSCAASGSTLNIDHIGWYRQAPGKERELVGVISSGAGPNYADSVKGRFTISRDNAKNTVYLQMNSLKPEDTAVYNCNAWIDYGSGLPQNYWGQGTQVTVXX

SEQ ID NO: 20 - protein, 116 aa, synthetic construct
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVXX

SEQ ID NO: 21 - protein, 120 aa, synthetic construct
EVQLVESGGGEVQPGGSLRLSCAASGSTLNIDHIGWYRQAPGKERELVGVISSGAGPNYAESVKGRFTISRDNAKNTVYLQMSSLRAEDTAVYYCNAWIDYGSGLPQNYWGQGTLVTVXX

SEQ ID NO: 22 - protein, 292 aa, synthetic construct
METDTLLLWVLLLWVPGSTGQVTLRESGGGLVQAGGSLRLSCKGSGRAINTYAMGWFRQAPGKEREFVAAISWNGGHTRYADSVQGRFAISRDNADNTMYLQMNSLKPEDTAVYHCAAYSDYHRIATMEADADSWGQGTQVTVSSGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 23 - protein, 287 aa, synthetic construct
METDTLLLWVLLLWVPGSTGEVQLVESGGGEVQPGGSLRLSCAASGRTFSGYIMGWFRQAPGKERELVARISGNNLSTEYAESVKGRFTISRDNAKNTLYLQMSSLRAEDTAVYYCAAEYDYSSGDFVYWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 24 - protein, 285 aa, synthetic construct
METDTLLLWVLLLWVPGSTGEVQLVESGGGEVQPGGSLRLSCAASGSGFSASLMSWHRQAPGKQRDLVASITRDGRANYVESVKGRFTISRDNAKNTLYLQMSSLRAEDTAVYYCHAYSFDYPIRSYWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 25 - protein, 285 aa, synthetic construct
METDTLLLWVLLLWVPGSTGEVQLVESGGGEVQPGGSLRLSCAASGFTFSSYAMGWFRQAPGKGREWVAAITTSGDTTYYAESVKGRFTISRDNAKNTVYLQMSSLRAEDTAVYYCAAHRGGGVIDYWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 26 - protein, 288 aa, synthetic construct
METDTLLLWVLLLWVPGSTGEVQLVESGGGEVQPGGSLRLSCAASGSIFSISIMGWYRQAPGKERELVASTTSSGTTNYVESVKGRFTISRDNAKNTLYLQMSSLRAEDTAVYYCHAYIATTTDRGYRGYWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 27 - protein, 291 aa, synthetic construct
METDTLLLWVLLLWVPGSTGQVQLVQSGGGLVQAGGSLRLSCAASGSISSYNVVGWYRQLSGNERGGRTMVAQINAYGDTNYANAVVGRFTISRDDAKNTVYLHMSNLKPEDTGVYYCNGQRMLENYTYRDQSWGQGTQVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 28 - protein, 283 aa, synthetic construct
METDTLLLWVLLLWVPGSTGQVQLVQSGGGLVQAGGSLTLSCAASRSSGIDVMGWYRQAPGKERELVAEISGVGDTNYAASLADRFTVSRDNAKNTVYLQMKNLKPEDTAVYYCNAHSFLDLVGAWGQGTQVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 29 - protein, 287 aa, synthetic construct
METDTLLLWVLLLWVPGSTGEVQLVQSGGGSVQVGGSLRLSCAASGSTLNIDHIGWYRQAPGKERELVGVISSGAGPNYADSVKGRFTISRDNAKNTVYLQMNSLKPEDTAVYNCNAWIDYGSGLPQNYWGQGTQVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 30 - protein, 283 aa, synthetic construct
METDTLLLWVLLLWVPGSTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 31 - protein, 287 aa, synthetic construct
METDTLLLWVLLLWVPGSTGEVQLVESGGGEVQPGGSLRLSCAASGSTLNIDHIGWYRQAPGKERELVGVISSGAGPNYAESVKGRFTISRDNAKNTVYLQMSSLRAEDTAVYYCNAWIDYGSGLPQNYWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 32 - protein, 617 aa, synthetic construct
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGQVTLRESGGGLVQAGGSLRLSCKGSGRAINTYAMGWFRQAPGKEREFVAAISWNGGHTRYADSVQGRFAISRDNADNTMYLQMNSLKPEDTAVYHCAAYSDYHRIATMEADADSWGQGTQVTVSSGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 33 - protein, 612 aa, synthetic construct
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGEVQLVESGGGEVQPGGSLRLSCAASGRTFSGYIMGWFRQAPGKERELVARISGNNLSTEYAESVKGRFTISRDNAKNTLYLQMSSLRAEDTAVYYCAAEYDYSSGDFVYWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 34 - protein, 610 aa, synthetic construct
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGEVQLVESGGGEVQPGGSLRLSCAASGSGFSASLMSWHRQAPGKQRDLVASITRDGRANYVESVKGRFTISRDNAKNTLYLQMSSLRAEDTAVYYCHAYSFDYPIRSYWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 35 - protein, 610 aa, synthetic construct
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGEVQLVESGGGEVQPGGSLRLSCAASGFTFSSYAMGWFRQAPGKGREWVAAITTSGDTTYYAESVKGRFTISRDNAKNTVYLQMSSLRAEDTAVYYCAAHRGGGVIDYWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 36 - protein, 613 aa, synthetic construct
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGEVQLVESGGGEVQPGGSLRLSCAASGSIFSISIMGWYRQAPGKERELVASTTSSGTTNYVESVKGRFTISRDNAKNTLYLQMSSLRAEDTAVYYCHAYIATTTDRGYRGYWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 37 - protein, 616 aa, synthetic construct
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGQVQLVQSGGGLVQAGGSLRLSCAASGSISSYNVVGWYRQLSGNERGGRTMVAQINAYGDTNYANAVVGRFTISRDDAKNTVYLHMSNLKPEDTGVYYCNGQRMLENYTYRDQSWGQGTQVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 38 - protein, 608 aa, synthetic construct
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGQVQLVQSGGGLVQAGGSLTLSCAASRSSGIDVMGWYRQAPGKERELVAEISGVGDTNYAASLADRFTVSRDNAKNTVYLQMKNLKPEDTAVYYCNAHSFLDLVGAWGQGTQVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 39 - protein, 612 aa, synthetic construct
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGEVQLVQSGGGSVQVGGSLRLSCAASGSTLNIDHIGWYRQAPGKERELVGVISSGAGPNYADSVKGRFTISRDNAKNTVYLQMNSLKPEDTAVYNCNAWIDYGSGLPQNYWGQGTQVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 40 - protein, 608 aa, synthetic construct
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 41 - protein, 612 aa, synthetic construct
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGEVQLVESGGGEVQPGGSLRLSCAASGSTLNIDHIGWYRQAPGKERELVGVISSGAGPNYAESVKGRFTISRDNAKNTVYLQMSSLRAEDTAVYYCNAWIDYGSGLPQNYWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 42 - protein, 301 aa, synthetic construct
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 120 aa, synthetic construct
EVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKP

SEQ ID NO: 44 - protein, 121 aa, synthetic construct
EVQLVESGGGEVQPGGSLRLSCAASGFTFSVYDMNWYRQAPGKEREWVSGISNNGFSTAYAESVKGRFTISRDNAKNTLYLQMSSLRAEDTAVYYCNLDVHGRVGAQGDYWGQGTLVTVKP

SEQ ID NO: 45 - protein, 129 aa, synthetic construct
EVQLVESGGGEVQPGGSLRLSCAASGFTFDDYAIGWFRQAPGKEREGVSTISSSDGSTYYAESVKGRFTISRDNAKNTVYLQMSSLRAEDTAVYYCALREGLPLLLDTLWRQPVEYDYWGQGTQVTVKP

SEQ ID NO: 46 - protein, 116 aa, synthetic construct
EVQLVESGGGEVQPGGSLRLSCAASGLLFSIYDMNWYRQAPGKEREWVAGITNNGYSTAYAESVKGRFTISRDNAKNTVYLQMSSLRAEDTAVYYCHTDEWGREYWGQGTLVTVKP

SEQ ID NO: 47 - protein, 112 aa, synthetic construct
EVQLVESGGGEVQPGGSLRLSCAASGRTFEYDDMVWFRQAPGKEREEVAGISWSGGSIYYAESVKGRFSISRDNAKNTVYLEMNRLKPEDTAVYYCNVGSYWGQGTLVTVKP

SEQ ID NO: 48 - protein, 119 aa, synthetic construct
EVQLVESGGGEVQPGGSLRLSCAASGFTFRINDMGWYRQAPGKECEMVAGISNNGFSTAYAESVKGRFTISRDNAKNTVYLQMSSLRAEDTAVYYCHRTDEGAAHACSRGQGTLVTVKP

SEQ ID NO: 49 - protein, 120 aa, synthetic construct
EVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVXX

SEQ ID NO: 50 - protein, 121 aa, synthetic construct
EVQLVESGGGEVQPGGSLRLSCAASGFTFSVYDMNWYRQAPGKEREWVSGISNNGFSTAYAESVKGRFTISRDNAKNTLYLQMSSLRAEDTAVYYCNLDVHGRVGAQGDYWGQGTLVTVXX

SEQ ID NO: 51 - protein, 129 aa, synthetic construct
EVQLVESGGGEVQPGGSLRLSCAASGFTFDDYAIGWFRQAPGKEREGVSTISSSDGSTYYAESVKGRFTISRDNAKNTVYLQMSSLRAEDTAVYYCALREGLPLLLDTLWRQPVEYDYWGQGTQVTVXX

SEQ ID NO: 52 - protein, 116 aa, synthetic construct
EVQLVESGGGEVQPGGSLRLSCAASGLLFSIYDMNWYRQAPGKEREWVAGITNNGYSTAYAESVKGRFTISRDNAKNTVYLQMSSLRAEDTAVYYCHTDEWGREYWGQGTLVTVXX

SEQ ID NO: 53 - protein, 112 aa, synthetic construct
EVQLVESGGGEVQPGGSLRLSCAASGRTFEYDDMVWFRQAPGKEREEVAGISWSGGSIYYAESVKGRFSISRDNAKNTVYLEMNRLKPEDTAVYYCNVGSYWGQGTLVTVXX

SEQ ID NO: 54 - protein, 119 aa, synthetic construct
EVQLVESGGGEVQPGGSLRLSCAASGFTFRINDMGWYRQAPGKECEMVAGISNNGFSTAYAESVKGRFTISRDNAKNTVYLQMSSLRAEDTAVYYCHRTDEGAAHACSRGQGTLVTVXX

SEQ ID NO: 55 - protein, 93 aa, Homo sapiens
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 56 - protein, 93 aa, Homo sapiens [sequence omitted]

SEQ ID NO: 57 - protein, 107 aa, Homo sapiens
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 58 - protein, 107 aa, Homo sapiens
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 59 - DNA, 10 bp, synthetic construct
  source 1..10: mol_type = other DNA
gccrccatgg